SEQUENCE LISTING (22 sequences)

SEQ ID NO: 1 - DNA, 2876 bp, homo sapiens
  CDS (613)...(1914)
gtcaccacgcgtccgcggacgcgcgtccggcgcccagcggagtaggggctgcgcttggggtttgctgaagctggctgcctctcccactccccttttgggtgcaaagcgccgctagcgggaagacgggggccgggcggggacaggggcacctgcgtagctggactgagagcctgcgcccagcttacatcgaccccacccggccccggcccgacccgacgcgacccgatccgatccgatcccattccatccgttcctcgtctcctcccggtctgacccgttgcccggccgtggttcgccacaccaggcatccaaagctgaggtcgctcctacggcctgggctcgccttcgctttagagatgtttggcctcttccctcccaaacagcccatcttcaaaacctggactcttggactggcacctggccacctttccctctaccaagactccacttccgtcttacccacttcttcctcagattcttggtaccccctgggttggagactgctcattttccttccaaattaatcccagaccccctaaaatattgacaaccttgacaaccccccaaccgaggagccagactttgttttggactaacttccatagccctgtcatggaggcagtgtacctggtagtgaatgggttgggcctggtgctggacgtgctgaccttggtgttggacctcaacttcctgctggtgtcctccctcctggcttccctggcctggctcctggccttcgtctacaacctgccgcacacggtactgactagtcttctgcacttgggccgcggagtcttgctttcattgctggccttgatcgaagccgtggtccggttcacatgtgggggcttgcaggccttgtgtactctgctgtatagctgctgctctggcctagagagcctaaagctcctggggcacctggcctctcatggggcactgcggagcagggagatactgcaccggggcgtcctcaatgtggtctccagtggccatgctttgctgcgccaggcctgtgacatctgtgccattgccatgagcctggtggcttatgtgatcaacagcctggtcaacatctgcctcatcggcactcagaacctcttttccctggtgctggccctgtgggatgcagtgaccgggcctctgtggaggatgacagacgtagtggctgccttcctagcccacatttccagcagtgctgtggccatggccatcctcctttggacaccctgccaactagccctggagctgctggcctcagctgcccgcctcctggccagctttgtgcttgtcaatctcactggcttggtgttgctagcttgtgtgctggcagtgacggtgactgtgttgcatccggacttcaccctgaggctggctacccaggcactcagccagctccatgcccggccatcctaccaccgtcttcgagaggatgtcatgcggctctctcgcctagcactgggctcagaggcctggcgccgagtctggagccgcagtctgcagctggcgagttggccaaaccggggaggggcacctggagctccccagggtgaccctatgagggtattctcagttaggacccggagacaggacactcttcctgaagcggggcgcagatcagaggcagaagaggaggaggccaggaccatcagagtgacacctgtcaggggccgagagaggctcaatgaggaggagcctccaggtgggcaagacccttggaaattgctgaaggagcaagaggagcggaagaagtgtgtcatctgccaggaccagagcaagacagtgttgctcctgccctgccggcatctgtgcctgtgccaggcctgcactgaaatcctgatgcgccaccccgtctaccaccgcaattgcccgctctgccgccggggcatcctgcagaccctcaatgtctacctctgaagcctccttccctgcctgcccacccctccatgctccacgcaggcactcacgctaggacagcattaacacctcatctccgggtcctggtctgaatcccctcctacccctgtggccatcctgccatacatccaggacattgagttggaagactatgatctgggtgggggcaggataacatggcttctctttacccagtgggtcccttcgatgctgagggtggtgagtatgtcactatgcaagggccctgagactatttgctgtgggctctcctccagcctgcccagggcccacccagatgcctctggggttacccctgtctgcttctggtttttctgttggagatctataggtccttttcctgcctccttcacatttcctccccagcttttgcggccacaacacatcagtgtcatttgggtgttttggcaactcaggggccttcggatgatcttaaacctttgtgttcagccagagcccctgtgccctggtaggcgttggggttagtatctctcgggtgccctcagagccacctctgcctgtgatcgtctgatgaggctccctcccaacctgatccaaaagccagtctcaggagtttacccctgggatgggggatgcatctgcacctgactttggggccacgtgccctgtggcaccccagctcactgggagtctcaggagggataaccggatttctgctctttcccctgtcactcccacatcacacagaaaaatggcattcctctctgtctctccctggcatggagagggcagactgtgcacatttcactagggtccaaatacagaagggcccagggcccaggggcttgcagcttcgtgaggggtctctggcccagtttccaatgaataaagttctcttgacagctcaaaaaaaaaaaaaaaaaaaaaaaaaaaaaaaaggccgcaagcttatcaatttcgacctatactgggtcgtattacaa

SEQ ID NO: 2 - protein, 433 aa, homo sapiens
MEAVYLVVNGLGLVLDVLTLVLDLNFLLVSSLLASLAWLLAFVYNLPHTVLTSLLHLGRGVLLSLLALIEAVVRFTCGGLQALCTLLYSCCSGLESLKLLGHLASHGALRSREILHRGVLNVVSSGHALLRQACDICAIAMSLVAYVINSLVNICLIGTQNLFSLVLALWDAVTGPLWRMTDVVAAFLAHISSSAVAMAILLWTPCQLALELLASAARLLASFVLVNLTGLVLLACVLAVTVTVLHPDFTLRLATQALSQLHARPSYHRLREDVMRLSRLALGSEAWRRVWSRSLQLASWPNRGGAPGAPQGDPMRVFSVRTRRQDTLPEAGRRSEAEEEEARTIRVTPVRGRERLNEEEPPGGQDPWKLLKEQEERKKCVICQDQSKTVLLLPCRHLCLCQACTEILMRHPVYHRNCPLCRRGILQTLNVYL

SEQ ID NO: 3 - DNA, 1302 bp, homo sapiens
  CDS (1)...(1302)
atggaggcagtgtacctggtagtgaatgggttgggcctggtgctggacgtgctgaccttggtgttggacctcaacttcctgctggtgtcctccctcctggcttccctggcctggctcctggccttcgtctacaacctgccgcacacggtactgactagtcttctgcacttgggccgcggagtcttgctttcattgctggccttgatcgaagccgtggtccggttcacatgtgggggcttgcaggccttgtgtactctgctgtatagctgctgctctggcctagagagcctaaagctcctggggcacctggcctctcatggggcactgcggagcagggagatactgcaccggggcgtcctcaatgtggtctccagtggccatgctttgctgcgccaggcctgtgacatctgtgccattgccatgagcctggtggcttatgtgatcaacagcctggtcaacatctgcctcatcggcactcagaacctcttttccctggtgctggccctgtgggatgcagtgaccgggcctctgtggaggatgacagacgtagtggctgccttcctagcccacatttccagcagtgctgtggccatggccatcctcctttggacaccctgccaactagccctggagctgctggcctcagctgcccgcctcctggccagctttgtgcttgtcaatctcactggcttggtgttgctagcttgtgtgctggcagtgacggtgactgtgttgcatccggacttcaccctgaggctggctacccaggcactcagccagctccatgcccggccatcctaccaccgtcttcgagaggatgtcatgcggctctctcgcctagcactgggctcagaggcctggcgccgagtctggagccgcagtctgcagctggcgagttggccaaaccggggaggggcacctggagctccccagggtgaccctatgagggtattctcagttaggacccggagacaggacactcttcctgaagcggggcgcagatcagaggcagaagaggaggaggccaggaccatcagagtgacacctgtcaggggccgagagaggctcaatgaggaggagcctccaggtgggcaagacccttggaaattgctgaaggagcaagaggagcggaagaagtgtgtcatctgccaggaccagagcaagacagtgttgctcctgccctgccggcatctgtgcctgtgccaggcctgcactgaaatcctgatgcgccaccccgtctaccaccgcaattgcccgctctgccgccggggcatcctgcagaccctcaatgtctacctctga

SEQ ID NO: 4 - protein, 54 aa, Artificial Sequence
  Pfam consensus sequence
CPICLTTFDLDEPKPFKEPVLLPCGHSFCSKCIVELLRLSQNSKNNSVYKCPLC

SEQ ID NO: 5 - DNA, 2810 bp, homo sapiens
  CDS (744)...(1955)
  misc_feature 344, 345, 346, 347, 348, 349, 350, 351, 352, 353, 354,355,356, 357, 358, 359, 360, 361, 362, 363, 364, 365, 366, 367,368, 369, 370, 371, 372, 373, 374, 375, 376, 377, 378, 379,380,381, 382, 383, 384, 385, 386, 387, 388, 389, 390: n = A,T,C or G
  misc_feature 391, 392, 393, 394, 395, 396, 397, 398, 399, 400, 401,402,403, 404, 405, 406, 407, 408, 409, 410, 411, 412, 413, 414,415, 416, 417, 418, 419, 420, 421, 422, 423, 424, 425, 426, 427,428, 429, 430, 431, 432, 433, 434, 435, 436, 437: n = A,T,C or G
  misc_feature 438, 439, 440, 441, 442, 443, 444, 445, 446, 447, 448,449, 450, 451: n = A,T,C or G
cgcctgcgcagggcagcggcccgcggggcggaggctttataatcacttcgtcgttgccgctcggcttctatcgccgggagggcggttgaggcggtggtggcggcgtcggcggcggccggcgctggctgaggggcgctgaggcgggagctgtggcgctgggcgcccctggctcctcggcctctgccggccatgggctccgagaaggactccgagtcgccgcgctccacatcgctacatgcggccgcacccgaccctaagtgccgcagcggcggccggcgccggcgcctcaccttgcacagcgtcttttttgcctcggcccgcggccgccgcgcccgggccaagcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgcaggccgagccgccgccccggcggcggcggagcctgggttcgacgatgaggaggcggcggagggcggtggcccgggcgcggaggaggtggagtgtccgctgtgcctggtgcggctgccgcctgagcgggccccgcgcctcctcagctgtccgcaccgctcgtgccgggactgcctccgccactacctgcgcctggagataagcgagagcagggtgcccatcagctgccccgagtgcagcgagcgactcaacccgcacgacatccgcttgctgctcgccgacccgccgcttatgcacaagtacgaggagttcatgctgcgccgctacctagcctcggaccccgactgccgctggtgcccggccccggactgcggttatgctgttattgcctatggctgtgccagctgcccgaagctaacttgtgagagggaaggttgccagactgagttctgctaccactgcaagcagatatggcatccaaatcagacatgcgatatggcccgtcaacagagggcccagactttacgagttcggaccaaacacacttcaggtctcagttatgggcaagaatctggaccagatgacatcaagccatgcccacgatgcagtgcatacattatcaagatgaatgatggaagctgtaatcacatgacctgtgcagtgtgtggctgtgaattctgttggctttgtatgaaagagatctcagacttgcattacctcagcccctctggctgtacattctggggcaagaagccatggagccgtaagaagaaaattctttggcagctgggcacgttgattggtgctccagtggggatttctctcattgatggcattgccattcctgccatggtcattggcattcctgtttatgttggaaggaagattcacagcaggtatgagggaaggaaaacctccaaacacaagaggaatttggctatcactggaggagtgactttgtcggtcattgcatccccagttattgctgcagttagtgttggtattggtgtccccattatgctggcatatgtttatggggttgtgcccatttctctttgtcgtggaggtggctgtggagttagcacagccaacggaaaaggagtgaaaattgaatttgatgaagatgatggtccaatcacagtggcagatgcctggagagccctcaagaatcccagcattggggaaagcagcattgaaggcctgactagtgtattgagcactagtggaagccctacagatggacttagtgttatgcaaggtccttacagcgaaacggccagctttgcagccctctcagggggcacgctgagtggcggcattctctccagtggcaagggaaaatatagcaggttagaagttcaagccgatgtccaaaaggaaattttccccaaagacacagccagtcttggtgcaattagtgacaacgcaagcactcgtgctatggccggttccataatcagttcctacaacccacaggacaggtttagcatgatccatgcatgactcagcaaagtggattttgtctccacagagaatgcaacaatatggaaatccaagtggacattgaagccaaaccaagccactatcagctggtgagtggaagcagcacggaggactcgctccatgttcatgctcagatggcagagaatgaagaagaaggtagtggtggcggaggcagtgaagaggatcccccctgcagacaccaaagctgtgaacagaaagactgcctggccagcaaaccttgggacatcagcctggcccagcctgaaagcatccgcagtgacctagagagttctgatgcacagtcagacgatgtgccagacatcacctcagatgagtgtggctccccccgctcccatactgcagcctgcccctcgacccccagagcccaaggtgcaccgagcccaagtgcccatatgaacctctctgccctagccgagggacaaactgtcttgaagccagaaggtggagaagccagagtatgaagtggaatgaatgctcctgttctgagaagcacacttgtaactgcatcttttggaatttttttttttttttttccaaggggtagagatttatgtattttatttcacagattctctggtcacaggtttttgcccagggaaattctgagaaattcacaatttcttaccagataaaacatgaaaagtttgccgttagttcccctcccctcccctccctctttttagttttaatttattggttaaactgatggcagcaatccatgaggtgtgtcaaagagtgtacatatgtatgtgtgtatattgaatgctaaacatattactgaaagacacattttaataaagatttctgtcataattcaaaaaaaaaaaaaaaaaa

SEQ ID NO: 6 - protein, 403 aa, homo sapiens
MHKYEEFMLRRYLASDPDCRWCPAPDCGYAVIAYGCASCPKLTCEREGCQTEFCYHCKQIWHPNQTCDMARQQRAQTLRVRTKHTSGLSYGQESGPDDIKPCPRCSAYIIKMNDGSCNHMTCAVCGCEFCWLCMKEISDLHYLSPSGCTFWGKKPWSRKKKILWQLGTLIGAPVGISLIDGIAIPAMVIGIPVYVGRKIHSRYEGRKTSKHKRNLAITGGVTLSVIASPVIAAVSVGIGVPIMLAYVYGVVPISLCRGGGCGVSTANGKGVKIEFDEDDGPITVADAWRALKNPSIGESSIEGLTSVLSTSGSPTDGLSVMQGPYSETASFAALSGGTLSGGILSSGKGKYSRLEVQADVQKEIFPKDTASLGAISDNASTRAMAGSIISSYNPQDRFSMIHA

SEQ ID NO: 7 - DNA, 1212 bp, homo sapiens
  CDS (1)...(1212)
atgcacaagtacgaggagttcatgctgcgccgctacctagcctcggaccccgactgccgctggtgcccggccccggactgcggttatgctgttattgcctatggctgtgccagctgcccgaagctaacttgtgagagggaaggttgccagactgagttctgctaccactgcaagcagatatggcatccaaatcagacatgcgatatggcccgtcaacagagggcccagactttacgagttcggaccaaacacacttcaggtctcagttatgggcaagaatctggaccagatgacatcaagccatgcccacgatgcagtgcatacattatcaagatgaatgatggaagctgtaatcacatgacctgtgcagtgtgtggctgtgaattctgttggctttgtatgaaagagatctcagacttgcattacctcagcccctctggctgtacattctggggcaagaagccatggagccgtaagaagaaaattctttggcagctgggcacgttgattggtgctccagtggggatttctctcattgatggcattgccattcctgccatggtcattggcattcctgtttatgttggaaggaagattcacagcaggtatgagggaaggaaaacctccaaacacaagaggaatttggctatcactggaggagtgactttgtcggtcattgcatccccagttattgctgcagttagtgttggtattggtgtccccattatgctggcatatgtttatggggttgtgcccatttctctttgtcgtggaggtggctgtggagttagcacagccaacggaaaaggagtgaaaattgaatttgatgaagatgatggtccaatcacagtggcagatgcctggagagccctcaagaatcccagcattggggaaagcagcattgaaggcctgactagtgtattgagcactagtggaagccctacagatggacttagtgttatgcaaggtccttacagcgaaacggccagctttgcagccctctcagggggcacgctgagtggcggcattctctccagtggcaagggaaaatatagcaggttagaagttcaagccgatgtccaaaaggaaattttccccaaagacacagccagtcttggtgcaattagtgacaacgcaagcactcgtgctatggccggttccataatcagttcctacaacccacaggacaggtttagcatgatccatgcatga

SEQ ID NO: 8 - protein, 72 aa, Artificial Sequence
  Pfam consensus sequence
EKYEKFMVRSYVEKNPDLKWCPGPDCSYAVRLTEVSSSTELAEPPRVECKKPACGTSFCFKCGAEWHAPVSC

SEQ ID NO: 9 - DNA, 5502 bp, homo sapiens
  CDS (803)...(2845)
ttaaactcccatgtgtgaggagtgtgcctccctgygccctctcagctctgaggctggycgtctttcggggtgttccttttggcaaatatacactgtaatcttgagtctaaatttatatgttgaaatgctaccttttttaaagtaagaagctaaataaaattattttactatcagtatcagtaaaaaaaaaaaaaaaagggcggccgcgccaccgccggagagggaggcccgagcgcaggagcctctggtggatgggtgcagcggcggcgggaggacgcggaagaggagccccgggggtagcggcggcgcgagcaggggcgcggggaccgggctgtctgaggtgcgcgccgcgctggggctcgcgctctacctgatcgcgctgcggacgctggtgcagctctcgctgcagcagctcgtgctacgcggggccgctggacaccgcggggagttcgacgcgctccaagccagggattatcttgaacacataacctccattggccccaggactacaggaagtccagaaaatgaaattctgaccgtgcactaccttttggaacagattaaactgattgaagtgcaaagcaacagccttcataagatttcagtagatgtacaacggcccacaggctcttttagcattgatttcttgggaggttttacaagctattatgacaacatcaccaatgttgtggtaaagctggaacccagagatggagcccagcatgctgtcttggctaattgtcattttgactcagtagcaaactcaccaggtgccagtgatgatgcagttagctgctcagtgatgctggaagtccttcgcgtcttgtcaacatcttcagaagccttgcatcatgctgtcatatttctctttaatggtgctgaggaaaatgtcttgcaagccagtcatggtttcattactcagcacccctgggctagcttgattcgtgcattcattaacctagaggcagcaggtgtaggagggaaagaacttgtattccaaacaggtcctgaaaatccttggttggttcaagcttatgtttcagcagctaaacacccttttgcttctgtggtggctcaggaggtttttcagagtggaatcattccttcagatactgactttcgtatctacagggattttgggaacattccaggaatagacttagcttttattgagaatggatacatttatcacaccaagtatgacacagcggacagaattctaacagattccattcagagagcaggtgacaacattttagcagttcttaagcatctagctacatctgatatgctggctgctgcttctaagtatcgacatggaaacatggtcttctttgatgtgctgggcctgtttgtcattgcctacccctctcgtattggctcaatcataaactacatggtggtaatgggtgttgttttgtacctgggcaaaaaatttttgcagcccaaacataagactggtaactacaagaaggacttcttgtgtggacttggcatcactttgatcagctggttcactagccttgttaccgttctcattatagcagtgttcatctctcttattggacagtctctctcatggtataaccacttctatgtctccgtttgtctgtatggaactgcaactgtagccaaaataatacttatacatactcttgcgaaaagattttattacatgaatgccagtgcccagtatctgggagaagtattttttgacatttcgctgtttgtccattgctgttttcttgttaccctcacttaccaaggactttgctcggcgtttattagtgctgtctgggtagcattcccattgctcacaaagctctgtgtgcataaggacttcaagcagcatggtgcccaaggaaaatttattgctttttaccttttggggatgtttattccttatctttatgcattgtacctcatctgggcagtatttgagatgtttacccctatcctcgggagaagtggttctgaaatcccacctgatgttgtgctggcatccattttggctggctgtacaatgattctctcgtcctattttattaacttcatctaccttgccaagagcacaaaaaaaaccatgctaactttaactttggtatgtgcaattacattcctccttgtttgcagtggaacattttttccatatagctccaatcctgctaatccgaagccaaagagagtgtttcttcagcatatgactagaacattccatgacttggaaggaaatgcagttaaacgggactctggaatatggatcaatgggtttgattatactggaatttctcacataacccctcacattcctgagatcaatgatagtatccgagctcactgtgaggagaatgcacctctttgtggttttccttggtatcttccagtgcactttctgatcaggaaaaactggtatcttcctgccccagaagtttctccaagaaatcctcctcatttccgactcatatccaaagaacagacaccttgggattctataaaattgacttttgaagcaacaggaccaagccatatgtccttctatgttcgagcccacaaagggtcaacactttctcagtggtctcttggcaatggcaccccagtcacaagtaaaggaggagactactttgtcttttactcccatggactccaggcctctgcatggcagttctggatagaagtgcaggtttcagaagaacatcctgaaggaatggtcaccgtggccattgctgcccactatctgtctggggaagacaagagatcccctcaactggatgctctgaaggaaaagttcccagattggacatttccctctgcctgggtgtgcacctacgatctctttgtattttaatcttgtggatgagctctaagtacatgcccagtggatactccatgtgacatggtttctccctatgttacgtggatgtttgtaacgtaagtcaatgaattttaatgatcatatgttcaaagagctttctgggttaacgcttttcagggccaagcactataagggtttagctgtggcgcagtgatgcatggcctgttgacacttgaaaatgccagtcttttggcacttcagcacatgtgggtactgccactacacacacgtcattttatatgaccttaaggacaaagccaacaatccacttcaatagctgcccctttaggatcaagaaagatgtacactgtcagagcattgttaatgagacaaaagttgtttccaatttaagccccaaaaccatttgttgtattagtggatggtgggtaaaatatcattcactgaggtaatgattccccttgagaatataactctgtgtaggtcactggaaagtgattgccatagggctgggagagaagcattgcactcttgaggctgtagcctgtgtcaagctgtttcttcaggcagcctctcaaatgtgctttgtctctctgtgctgaggcctggaccctgtgctgagctggtgactcactgtcctgacaagtggacacacagatgcactgctgtgctgctttcctgaggtggttttctatgcctgttttcctctgaaacatgtctgttacccctctccatcttaccaagttgaaaaggggaatatttggccacatacccctctggttttcgtaggttcttttggttcagaatattgtttgtgccagtacatgaccttaacttccttcctcagagcactgagctgccatctgggctattctggggtagaaggaaggctgggagtggtgggaattttataaatatttattctcttttctttgtttcataggagtcttgtgttatacaaggttagtccttcatggtataatcttactgatgcactgggcctatctttttgttttccagccagttgaatagattagtttttctcagtaacttactatccagcagactggctttcctgagacttgaggttgtggcttatactggaatgagaccactgtacgtgtaggtggttcagatcctgcgtaatggcagcatgaggacttaaaaggtggttttcattttgaagatggctatgtagcttgtaaggtgtatcacagcagtacctctcatggctttttggttccagcagtgagggcattggtgagatcaatggtaaactgtgcaagctttctttttatcattaggaaatgtgaaacgttggacaaattttgagttttaacaaggacaaaaagttgaaagaaaaggcacagttaacaaaaaagggtggctagatttatcttgggtgatggaggaaatgagagaggaatgctcttgaaaggtggtctgtggatctgtctgaatagaaagagcacagtaagtatgcattgccggagaaaacgtccttgaagctgcttgtctcatgtgtatgatgtgctttttaaatcatgcccctcgttgcctgcctaatctgtgactccctaaaaactaactgggcccatgtagatggggctgcaaccagagctgaataacatgttaggctcacacatgcatcagcactgcacactggaatcattgctcttcctggactttgtagaaatcagtctcaagtgcttcaagagtctggctcctgctacttttatctgtcaggtagcacataaggtttgcagggtttatattttgtatagaatcacagttgtggagaaaaagtaataatttctcaatgaattttaaaaatgggcctattttctatccccgtggttcatctgatataattagtgttccctgtgaattccccccctctatgggaaggatgcctttactctttatcagtaataaattatgactgttttcatattgccttagggttatttccctgtgtaaaccattgtcttttgttttggttttctttagcattatgaagctttggtattgtacaaggtcagtagtaagatgctcactagtctcagggcttgtgtaatattctgggaggtcatttaaatgccagaaatggtcaagcaattatacacagtatttatgactctgttaagcataccgtttgtctgtcacattagtagattctgagattaaaaaaaatttttaaagagtgatcatttaaataatttctaaaagggtcttttcaagctctaacaaagtcactaacaaatgcattattttctacagaattagatgttagtagtacagtactgcatattcagggaaaaagtgtgaggaattgatttcaaaatagttcgttcttgtgtttgacctaagaatgattgtcgcatgaagtgtttgtttttacagtttagcatatataaacaaacatgataggattccttaagatgttaccacccagggggccacaagccagcctgctgtctcaggaagctgtagaaggagtgtttgtcaatttcttgtcactggtttgctgacttactgaggattaattgttgccttacaatgttactgaaataaactgtttaataaaaaaaaaaaaaaaaaaaaaaaaaaaaaaaaaaaaaaaaaaaaaaaaaaaaaaaaaaaaaaaaaaacaaaaaaaaaagggcggccgc

SEQ ID NO: 10 - protein, 680 aa, homo sapiens
MLEVLRVLSTSSEALHHAVIFLFNGAEENVLQASHGFITQHPWASLIRAFINLEAAGVGGKELVFQTGPENPWLVQAYVSAAKHPFASVVAQEVFQSGIIPSDTDFRIYRDFGNIPGIDLAFIENGYIYHTKYDTADRILTDSIQRAGDNILAVLKHLATSDMLAAASKYRHGNMVFFDVLGLFVIAYPSRIGSIINYMVVMGVVLYLGKKFLQPKHKTGNYKKDFLCGLGITLISWFTSLVTVLIIAVFISLIGQSLSWYNHFYVSVCLYGTATVAKIILIHTLAKRFYYMNASAQYLGEVFFDISLFVHCCFLVTLTYQGLCSAFISAVWVAFPLLTKLCVHKDFKQHGAQGKFIAFYLLGMFIPYLYALYLIWAVFEMFTPILGRSGSEIPPDVVLASILAGCTMILSSYFINFIYLAKSTKKTMLTLTLVCAITFLLVCSGTFFPYSSNPANPKPKRVFLQHMTRTFHDLEGNAVKRDSGIWINGFDYTGISHITPHIPEINDSIRAHCEENAPLCGFPWYLPVHFLIRKNWYLPAPEVSPRNPPHFRLISKEQTPWDSIKLTFEATGPSHMSFYVRAHKGSTLSQWSLGNGTPVTSKGGDYFVFYSHGLQASAWQFWIEVQVSEEHPEGMVTVAIAAHYLSGEDKRSPQLDALKEKFPDWTFPSAWVCTYDLFVF

SEQ ID NO: 11 - DNA, 2043 bp, homo sapiens
  CDS (1)...(2043)
atgctggaagtccttcgcgtcttgtcaacatcttcagaagccttgcatcatgctgtcatatttctctttaatggtgctgaggaaaatgtcttgcaagccagtcatggtttcattactcagcacccctgggctagcttgattcgtgcattcattaacctagaggcagcaggtgtaggagggaaagaacttgtattccaaacaggtcctgaaaatccttggttggttcaagcttatgtttcagcagctaaacacccttttgcttctgtggtggctcaggaggtttttcagagtggaatcattccttcagatactgactttcgtatctacagggattttgggaacattccaggaatagacttagcttttattgagaatggatacatttatcacaccaagtatgacacagcggacagaattctaacagattccattcagagagcaggtgacaacattttagcagttcttaagcatctagctacatctgatatgctggctgctgcttctaagtatcgacatggaaacatggtcttctttgatgtgctgggcctgtttgtcattgcctacccctctcgtattggctcaatcataaactacatggtggtaatgggtgttgttttgtacctgggcaaaaaatttttgcagcccaaacataagactggtaactacaagaaggacttcttgtgtggacttggcatcactttgatcagctggttcactagccttgttaccgttctcattatagcagtgttcatctctcttattggacagtctctctcatggtataaccacttctatgtctccgtttgtctgtatggaactgcaactgtagccaaaataatacttatacatactcttgcgaaaagattttattacatgaatgccagtgcccagtatctgggagaagtattttttgacatttcgctgtttgtccattgctgttttcttgttaccctcacttaccaaggactttgctcggcgtttattagtgctgtctgggtagcattcccattgctcacaaagctctgtgtgcataaggacttcaagcagcatggtgcccaaggaaaatttattgctttttaccttttggggatgtttattccttatctttatgcattgtacctcatctgggcagtatttgagatgtttacccctatcctcgggagaagtggttctgaaatcccacctgatgttgtgctggcatccattttggctggctgtacaatgattctctcgtcctattttattaacttcatctaccttgccaagagcacaaaaaaaaccatgctaactttaactttggtatgtgcaattacattcctccttgtttgcagtggaacattttttccatatagctccaatcctgctaatccgaagccaaagagagtgtttcttcagcatatgactagaacattccatgacttggaaggaaatgcagttaaacgggactctggaatatggatcaatgggtttgattatactggaatttctcacataacccctcacattcctgagatcaatgatagtatccgagctcactgtgaggagaatgcacctctttgtggttttccttggtatcttccagtgcactttctgatcaggaaaaactggtatcttcctgccccagaagtttctccaagaaatcctcctcatttccgactcatatccaaagaacagacaccttgggattctataaaattgacttttgaagcaacaggaccaagccatatgtccttctatgttcgagcccacaaagggtcaacactttctcagtggtctcttggcaatggcaccccagtcacaagtaaaggaggagactactttgtcttttactcccatggactccaggcctctgcatggcagttctggatagaagtgcaggtttcagaagaacatcctgaaggaatggtcaccgtggccattgctgcccactatctgtctggggaagacaagagatcccctcaactggatgctctgaaggaaaagttcccagattggacatttccctctgcctgggtgtgcacctacgatctctttgtattttaa

SEQ ID NO: 12 - DNA, 2566 bp, homo sapiens
  CDS (194)...(2470)
raagggactagtcctgcaagtttaaacgaatttcactcctcttactacactatagggctcgagcggccgcccgggcaggtggggggcataggtgaaaaatagtaaagagaggaaagccagaaacagtgagccagggaacaaggggacggaagacaggatccaggctgtatctacggagagacacagaagggagatgctgctgctgccgctgctgctgctgctgccgccactagtcctcagggttgctgcaagccgatgtctacatgatgagacacagaagtctgtgagccttctcaggccccctttctcccaactcccctcaaaatctcgctcttcctccctcaccctccctagctcccgtgatcctcaacccctacgaatccaaagctgctatctaggagatcatatatcagatggagcttgggatcctgagggagaagggatgagagggggatcccgagccctggccgcagtgagagaggccactcagcgaatccaggctgttctagcagtccctccagtgcaaggacccctgcttctgagtcgagaccctgcacagtattgccacgctgtctggggagacccagatagcccaaactaccacaggtgcagcctcttgaacccaggatacaaaggagagagttgcctgggggcaaagattcctgacacccatcttcgcggttatgccttgtggccggagcagggtcccccacaactggtccagccagatgggcctggggtccaaaacactgattttctcctgtatgtgcgagttgctcacacttccaagtgccaccaagagacagtctcactctgttgcccaggctggagtacagcggcccaatcacagctcaccgcagccttgacctcctgggctcagccctctgtcatagcctatgctgcctgctgccagctggactcagaagacaggcccctcgctggtaccattgtctactgtgcccaacatctcaccagccccagcctcagccacagtgacatcgtcatggccacattacatgaattgctccatgccttgggtttctctggacagctcttcaagaaatggcgagactgcccctcaggattcagtgttagagagaactgttctacaaggcaacaagtgacaaggcaagatgagtggggacaactgcttctcaccaccccagctgttagcctcagcctggccaaacacttgggagtgtcgggggcttccctgggtgttcccttggaagaagaggagggccttctgtcctcgcactgggaggccagactactccagggttctttaatgactgctacctttgatggagcccagcgcactcgactcgacccaatcaccctcgctgccttcaaagactcaggctggtaccaggtcaaccacagcgctgcagaggagctgttgtggggccagggatctggcccagaatttggcttggtgaccacatgtgggactggctcctcagacttcttctgtactggcagtgggctgggctgccactacctgcacctggacaagggaagctgctcctcagaccccatgctggaaggctgccgcatgtacaagcccttagccaatgggagtgaatgctggaagaaggaaaacggattccctgctggggtggataatccccatggggagatctaccatccccagagccgttgcttctttgccaacctcacttcacagctgctccctggggataagcccaggcatccttctcttaccccacacctcaaggaagcagagctcatgggccgctgctacttacatcaatgcacagggaggggagcttacaaggtgcaggtggagggctcgccttgggtcccatgccttcctggaaaggttatacagatacctgggtactatggtcttctcttctgtccccggggtcggctgtgtcagactaatgaaggtatcaatgctgttacttccccacctgtgagtctttcaaccccagatccactattccagctctctttagaattagctgggcctccaggacactctctggggaaggaacagcaagaagggctagctgaagcagtactggaggctttggcgagcagaggcggcactggcaggtgctatttccatggcccatcaattaccactagcttggtgtttactgtgcatatgtggaagtcccctggctgccaagggccttcagttgctacactgcacaaggccctgactctgactctccagaaaaaacccctagaagtgtatcatggaggagccaactttaccacacaacccagcaagttgctggttacttcagaccataatccctccatgacccacctaaggctgtccatgggactctgcctaatgctgctaatcctggtgggtgtaatgggaaccacagcctaccagaaaagagccactcttcctgtgagaccatctgcctcttaccattcaccagagctccacagcacaagggtcccagttagaggaataagggaggtgtgatgttgcccagaacatgacagggggtaaggaagagaataatttcttgtgagacgactggatggaaaatctattgggtatacttaatttctactttct

SEQ ID NO: 13 - protein, 758 aa, homo sapiens
MLLLPLLLLLPPLVLRVAASRCLHDETQKSVSLLRPPFSQLPSKSRSSSLTLPSSRDPQPLRIQSCYLGDHISDGAWDPEGEGMRGGSRALAAVREATQRIQAVLAVPPVQGPLLLSRDPAQYCHAVWGDPDSPNYHRCSLLNPGYKGESCLGAKIPDTHLRGYALWPEQGPPQLVQPDGPGVQNTDFLLYVRVAHTSKCHQETVSLCCPGWSTAAQSQLTAALTSWAQPSVIAYAACCQLDSEDRPLAGTIVYCAQHLTSPSLSHSDIVMATLHELLHALGFSGQLFKKWRDCPSGFSVRENCSTRQQVTRQDEWGQLLLTTPAVSLSLAKHLGVSGASLGVPLEEEEGLLSSHWEARLLQGSLMTATFDGAQRTRLDPITLAAFKDSGWYQVNHSAAEELLWGQGSGPEFGLVTTCGTGSSDFFCTGSGLGCHYLHLDKGSCSSDPMLEGCRMYKPLANGSECWKKENGFPAGVDNPHGEIYHPQSRCFFANLTSQLLPGDKPRHPSLTPHLKEAELMGRCYLHQCTGRGAYKVQVEGSPWVPCLPGKVIQIPGYYGLLFCPRGRLCQTNEGINAVTSPPVSLSTPDPLFQLSLELAGPPGHSLGKEQQEGLAEAVLEALASRGGTGRCYFHGPSITTSLVFTVHMWKSPGCQGPSVATLHKALTLTLQKKPLEVYHGGANFTTQPSKLLVTSDHNPSMTHLRLSMGLCLMLLILVGVMGTTAYQKRATLPVRPSASYHSPELHSTRVPVRGIREV

SEQ ID NO: 14 - DNA, 2277 bp, homo sapiens
  CDS (1)...(2277)
atgctgctgctgccgctgctgctgctgctgccgccactagtcctcagggttgctgcaagccgatgtctacatgatgagacacagaagtctgtgagccttctcaggccccctttctcccaactcccctcaaaatctcgctcttcctccctcaccctccctagctcccgtgatcctcaacccctacgaatccaaagctgctatctaggagatcatatatcagatggagcttgggatcctgagggagaagggatgagagggggatcccgagccctggccgcagtgagagaggccactcagcgaatccaggctgttctagcagtccctccagtgcaaggacccctgcttctgagtcgagaccctgcacagtattgccacgctgtctggggagacccagatagcccaaactaccacaggtgcagcctcttgaacccaggatacaaaggagagagttgcctgggggcaaagattcctgacacccatcttcgcggttatgccttgtggccggagcagggtcccccacaactggtccagccagatgggcctggggtccaaaacactgattttctcctgtatgtgcgagttgctcacacttccaagtgccaccaagagacagtctcactctgttgcccaggctggagtacagcggcccaatcacagctcaccgcagccttgacctcctgggctcagccctctgtcatagcctatgctgcctgctgccagctggactcagaagacaggcccctcgctggtaccattgtctactgtgcccaacatctcaccagccccagcctcagccacagtgacatcgtcatggccacattacatgaattgctccatgccttgggtttctctggacagctcttcaagaaatggcgagactgcccctcaggattcagtgttagagagaactgttctacaaggcaacaagtgacaaggcaagatgagtggggacaactgcttctcaccaccccagctgttagcctcagcctggccaaacacttgggagtgtcgggggcttccctgggtgttcccttggaagaagaggagggccttctgtcctcgcactgggaggccagactactccagggttctttaatgactgctacctttgatggagcccagcgcactcgactcgacccaatcaccctcgctgccttcaaagactcaggctggtaccaggtcaaccacagcgctgcagaggagctgttgtggggccagggatctggcccagaatttggcttggtgaccacatgtgggactggctcctcagacttcttctgtactggcagtgggctgggctgccactacctgcacctggacaagggaagctgctcctcagaccccatgctggaaggctgccgcatgtacaagcccttagccaatgggagtgaatgctggaagaaggaaaacggattccctgctggggtggataatccccatggggagatctaccatccccagagccgttgcttctttgccaacctcacttcacagctgctccctggggataagcccaggcatccttctcttaccccacacctcaaggaagcagagctcatgggccgctgctacttacatcaatgcacagggaggggagcttacaaggtgcaggtggagggctcgccttgggtcccatgccttcctggaaaggttatacagatacctgggtactatggtcttctcttctgtccccggggtcggctgtgtcagactaatgaaggtatcaatgctgttacttccccacctgtgagtctttcaaccccagatccactattccagctctctttagaattagctgggcctccaggacactctctggggaaggaacagcaagaagggctagctgaagcagtactggaggctttggcgagcagaggcggcactggcaggtgctatttccatggcccatcaattaccactagcttggtgtttactgtgcatatgtggaagtcccctggctgccaagggccttcagttgctacactgcacaaggccctgactctgactctccagaaaaaacccctagaagtgtatcatggaggagccaactttaccacacaacccagcaagttgctggttacttcagaccataatccctccatgacccacctaaggctgtccatgggactctgcctaatgctgctaatcctggtgggtgtaatgggaaccacagcctaccagaaaagagccactcttcctgtgagaccatctgcctcttaccattcaccagagctccacagcacaagggtcccagttagaggaataagggaggtgtga

SEQ ID NO: 15 - protein, 13 aa, Artificial Sequence
  Pfam consensus sequence
GEGVSNTDFVLYV

SEQ ID NO: 16 - protein, 65 aa, Artificial Sequence
  Pfam consensus sequence
PGVLAWATTCQVFSDFGRPAVGVINIPAANITSRNHYDQLVTRVVTHEIAHALGFSVGLYTFFEE

SEQ ID NO: 17 - protein, 55 aa, Artificial Sequence
  Pfam consensus sequence
SHWKKRNAKDELMAGAAGSDAGYYSALTMAVFEDLGFYKADFSKAEDMPWGKNAG

SEQ ID NO: 18 - protein, 37 aa, Artificial Sequence
  Pfam consensus sequence
AALCANVKCDTATRTYSVQVYGSSGYYPCTPGLRVEL

SEQ ID NO: 19 - DNA, 1485 bp, homo sapiens
  CDS (255)...(1133)
acaggctgtatcttctcaaaatttcattgattgggctcaatgaagtcacctgcaacatrgtagtagagtagggctcccttttcacacgctttttggaaggcttcttcaagtcacattttccgttcggtcttcctttgccctgtgtttgcrgtcatcatgtgaggggctacctatgttcagcccaggcaacccacagggagagagggcagagcggggagatggcccctggtgagcactgaggctccccttcaaggatggcgctggcggctttgatgatcgccctcggcagcctcggcctccacacctggcaggcccaggctgttcccaccatcctgcccctgggcctggctccagacacctttgacgatacctatgtgggttgtgcagaggagatggaggagaaggcagcccccctgctaaaggaggaaatggcccaccatgccctgctgcgggaatcctgggaggcagcccaggagacctgggaggacaagcgtcgagggcttaccttgccccctggcttcaaagcccagaatggaatagccattatggtctacaccaactcatcgaacaccttgtactgggagttgaatcaggccgtgcggacgggcggaggctcccgggagctctacatgaggcactttcccttcaaggccctgcatttctacctgatccgggccctgcagctgctgcgaggcagtgggggctgcagcaggggacctggggaggtggtgttccgaggtgtgggcagccttcgctttgaacccaagaggctgggggactctgtccgcttgggccagtttgcctccagctccctggataaggcagtggcccacagatttggtaatgccaccctcttctctctaacaacttgctttggggcccctatacaggccttctctgtctttcccaaggagcgcgaggtgctgattcccccccatgaagtctttttggttaccagattctctcaggatggagcccagagcttggtgactctctggagctataatcagacctgtagccattttaactgcgcctatctgggtggggagaagaggcggggctgtgtgtctgcgccaggagccctgggaacgggtgaccttcatatgacgaagaggcacctccagcagccttgagaagcaagaacatggttccggacccagccctagcagccttctccccaaccaggatgttggcctggggaggccacagcagggctgagggaactctgctatgtgatggggacttcctgggacaagcaaggaaagtactgaggcagccacttgattgaacggtgttgcaatgtggagacatggagttttattgaggtagctacgtgattaaatggtattgcagtgtggadaaadgrramwwmmwgggacaagcaaggaaagtactgaggcagccacttgattgaacggtgttgcaatgtggagacatggagttttattgaggtagctacgtgattaaatggtattgcagtgtgga

SEQ ID NO: 20 - protein, 292 aa, homo sapiens
MALAALMIALGSLGLHTWQAQAVPTILPLGLAPDTFDDTYVGCAEEMEEKAAPLLKEEMAHHALLRESWEAAQETWEDKRRGLTLPPGFKAQNGIAIMVYTNSSNTLYWELNQAVRTGGGSRELYMRHFPFKALHFYLIRALQLLRGSGGCSRGPGEVVFRGVGSLRFEPKRLGDSVRLGQFASSSLDKAVAHRFGNATLFSLTTCFGAPIQAFSVFPKEREVLIPPHEVFLVTRFSQDGAQSLVTLWSYNQTCSHFNCAYLGGEKRRGCVSAPGALGTGDLHMTKRHLQQP

SEQ ID NO: 21 - DNA, 879 bp, homo sapiens
  CDS (1)...(879)
atggcgctggcggctttgatgatcgccctcggcagcctcggcctccacacctggcaggcccaggctgttcccaccatcctgcccctgggcctggctccagacacctttgacgatacctatgtgggttgtgcagaggagatggaggagaaggcagcccccctgctaaaggaggaaatggcccaccatgccctgctgcgggaatcctgggaggcagcccaggagacctgggaggacaagcgtcgagggcttaccttgccccctggcttcaaagcccagaatggaatagccattatggtctacaccaactcatcgaacaccttgtactgggagttgaatcaggccgtgcggacgggcggaggctcccgggagctctacatgaggcactttcccttcaaggccctgcatttctacctgatccgggccctgcagctgctgcgaggcagtgggggctgcagcaggggacctggggaggtggtgttccgaggtgtgggcagccttcgctttgaacccaagaggctgggggactctgtccgcttgggccagtttgcctccagctccctggataaggcagtggcccacagatttggtaatgccaccctcttctctctaacaacttgctttggggcccctatacaggccttctctgtctttcccaaggagcgcgaggtgctgattcccccccatgaagtctttttggttaccagattctctcaggatggagcccagagcttggtgactctctggagctataatcagacctgtagccattttaactgcgcctatctgggtggggagaagaggcggggctgtgtgtctgcgccaggagccctgggaacgggtgaccttcatatgacgaagaggcacctccagcagccttga

SEQ ID NO: 22 - protein, 296 aa, Artificial Sequence
  Pfam consensus sequence
MPALHFVLLLSVGLLLSTQALSSAIQQKDGLVKELVLDMAPNSFDDQYLGCVDRMEAKYLPQLLKEEFAANEVLAVGWESAKAKWQERKARGSVWGSLPYPSPPMGFKDEHGIALLAYTASSQEQTPLYREFNEAVREAGRSREDYLHHFHFKALHFYLTRALQLLRSSGGCQPGPCHVVYRGVRGLRFRPQGGGASVRFGQFTSSSLKKKVAQSSEFFFGQDTFFSIKTCLGVPIKAFSFFPSEEEVLIPPFEVFQVINTSRPTAGSAIILLSSKGKCSTYNCEYLKGKKTENCI

(19) United States
(12) Patent Application Publication
(10) Pub. No.: US 2002/0151696 A1
(43) Pub. Date: Oct. 17, 2002

(54) 84242,8035, 55304, 52999, and 21999, novel human proteins and methods of use thereof

(75) Inventor: Rajaschkar Bandaru, Watertown, MA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Millennium Pharmaceuticals, Inc.

(21) Appl. No.: 09/996,194

(22) Filed: Nov. 28, 2001

Related U.S. Application Data
(60) Provisional application No. 60/250,348, filed on Nov. 30, 2000.
(60) Provisional application No. 60/250,073, filed on Nov. 30, 2000.
(60) Provisional application No. 60/253,878, filed on Nov. 29, 2000.
(60) Provisional application No. 60/250,338, filed on Nov. 30, 2000.

(51) Int. Cl. C07H 21/02; C07H 21/04

(52) U.S. Cl. 536/23.1

(57) ABSTRACT

The invention provides isolated nucleic acids molecules, designated 8035, 84242, 55304, 52999, or 21999 nucleic acid molecules. The invention also provides antisense nucleic acid molecules, recombinant expression vectors containing 8035, 84242, 55304, 52999, or 21999 nucleic acid molecules, host cells into which the expression vectors have been introduced, and nonhuman transgenic animals in which an 8035, 84242, 55304, 52999, or 21999 gene has been introduced or disrupted. The invention still further provides isolated 8035, 84242, 55304, 52999, or 21999 proteins, fusion proteins, antigenic peptides and anti-8035, anti-84242, anti-55304, anti-52999, or anti-21999 antibodies. Diagnostic methods utilizing compositions of the invention are also provided.

### CROSS REFERENCE TO RELATED APPLICATIONS

This application claims the benefit of U.S. Provisional Application No. 60/250,348, filed Nov. 30, 2000; U.S. Provisional Application No. 60/250,073, filed Nov. 30, 2000; U.S. Provisional Application No. 60/253,878, filed Nov. 29, 2000; and U.S. Provisional Application No. 60/250,338, filed Nov. 30, 2000.

### FIELD OF THE INVENTION

The present invention relates to newly identified proteins, 8035 and 84242, human RING finger proteins; 55304, a human aminopeptidase; 52999, a human metallopeptidase; and 21999, a human ribosyltransferase. In particular, the invention relates to the following: (1) 8035 and 84242 RING finger polypeptides and polynucleotides, methods of detecting the 8035 and 84242 RING finger polypeptides and polynucleotides, and methods of diagnosing and treating 8035 and 84242 RING finger-related disorders; (2) 55304 aminopeptidase polypeptides and polynucleotides, methods of detecting the 5304 aminopeptidase polypeptides and polynucleotides, and methods of diagnosing and treating 5304 aminopeptidase-related disorders; (3) 52999 metallopeptidase polypeptides and polynucleotides, methods of detecting the 52999 metallopeptidase polypeptides and polynucleotides, and methods of diagnosing and treating 52999 metallopeptidase-related disorders; and (4) 21999 human ribosyltransferase polypeptides and polynucleotides, methods of detecting the 21999 human ribosyltransferase polypeptides and polynucleotides, and methods of diagnosing and treating 21999 human ribosyltransferase-related disorders. Also provided are vectors, host cells, and recombinant methods for making and using the novel molecules.

### BACKGROUND OF THE INVENTION

RING Finger Proteins. Targeted protein proteolysis is increasingly understood to be an important general mechanism by which cells regulate protein levels and, consequently, their functions at specific times. In eukaryotic cells, the main mechanism for such control involves the specific covalent modification by polyubiquitin, which labels target proteins for proteolysis and subsequent degradation. There are many known examples of such events, and ubiquitination is now recognized as a major mechanism for cellular regulation (for review see: Freemont, P. S. (2000) Current Biology 10:R84-R87; Joazeiro and Weissman (2000) Cell 102:549-552; Jackson et al. (2000) Trends in Cell Biology 10:429-439).

Protein ubiquitination begins with the formation of a thiol-ester linkage between ubiquitin and the ubiquitin activating enzyme (E1). Ubiquitin is then transferred to a ubiquitin conjugating enzyme (E2), again through a thiol-ester linkage. The ubiquitin ligases (E3's), which are primarily responsible for providing specificity to the ubiquitin conjugation, interact with both E2 and substrate to promote ubiquitination. The E3 enzymes are thought to be the least conserved component of the ubiquitination pathway.

Recent studies indicate that E3's can be divided into two distinct protein classes; those containing a HECT domain and those containing a RING finger domain. The RING finger class of E3 ubiquitin ligases can be further grouped into the SCF, VBC and anaphase-promoting complexes, and single-polypeptide RING finger E3 enzymes. The RING finger motif and its variants have been found in more than 200 eukaryotic proteins, but interestingly not in any prokaryotic protein. Perhaps the most famous RING finger protein is BRCA1, the product of a breast cancer-associated gene. Point mutations within the RING finger domain of BRCA1 predispose females having the mutations to breast cancer. Other well-known family members include the protooncogene products Cbl, BMI-1, and PML; the immunoglobulin gene recombination enzyme RAG1; the Rbx1 component of the von Hippel Lindau (VHL) tumor suppressor complex; and the p53 regulator MDM2, to name but a few.

RING finger proteins play pivotal roles in diverse cellular processes and are implicated in contributing to disease. The biological roles of RING finger proteins include regulation of cellular proliferation, apoptosis, the cell cycle, cellular signaling, transcription, DNA repair, degradation from the endoplasmic reticulum (ER), and photomorphogenesis. In addition, RING mutations in the RING finger protein, Parkin, are associated with autosomal juvenile parkinsonism.

The tumor suppressor BRCA1 provides an example where loss of RING finger function is associated with dysregulated growth and malignancy, in the form of familial breast and ovarian cancer. Another example where loss of RING finger function is associated with malignancy is in the case of VHL disease. The RING finger protein Rbx1 is a component of the E3 complex that includes the VHL tumor suppressor protein, and VHL mutations that prevent assembly of this E3 are associated with the malignancies of VHL disease, perhaps due to the stabilization of proteins such as hypoxia inducing factor 1 alpha.

The influence of RING finger E3 ubiquitin ligases on the balance between cellular proliferation and apoptosis is demonstrated by the following examples. First, Mdm2 is a RING finger E3 ubiquitin ligase that functions as a regulator of the tumor suppressor, p53. The regulation of p53 by Mdm2 has been demonstrated to depend on the RING finger domain of Mdm2, thus, implicating this RING finger E3 ubiquitin ligase as a critical regulator of cellular proliferation. Second, the E3 ubiquitin ligase activity of a group of RING finger containing proteins known as Inhibitors of Apoptosis (IAP's) has been demonstrated to be the activity responsible for IAP auto-ubiquitination, degradation, and progression toward cell death in response to apoptotic stimuli (Yang et al. (2000) Science 288:874-877). Thus, these E3 ubiquitin ligases play a crucial role in the regulation of apoptosis.

One example of the clearly established role of RING finger proteins in the regulation of the cell cycle is that mitotic cyclins are targeted for degradation by ubiquitination mediated by the APC (or cyclosome) that includes the small RING finger protein, Apc11p.

The RING finger E3 ubiquitin ligases' role in the secretory pathway is through regulation of the disposal of membrane proteins from the endoplasmic reticulum (ER). One example of the impact of this key role in the secretory pathway is the RING finger E3 facilitation of disposal of a membrane protein from the ER contributing to the pathogenesis of AIDS. Beta TrCP, a RING finger protein that targets beta-catenin and IₖₐₚₚₐBₐₗₚₕₐ for ubiquitination, also targets Vpu-bound CD4 for degradation, resulting in an increase in the amount of HIV Env protein available for virus production.

Accordingly, RING finger proteins are a major target for drug action and development. Therefore, it is valuable to the field of pharmaceutical development to identify and characterize previously unknown RING finger proteins. The present invention advances the state of the art by providing two previously unidentified human RING finger proteins.

Aminopeptidases. Aminopeptidases are a group of widely-distributed exopeptidases that catalyze the hydrolysis of amino acid residues from the amino-terminus of peptide substrates. Members of this enzyme family are found throughout the animal and plant kingdoms, and are found subcellularly in organelles, in the cytoplasm, and as membrane components. Aminopeptidases function in many cellular processes, including protein maturation, the regulation of hormone levels (including vasopressin and noradrenaline levels), the regulation of the renin-angiotensin system, and in cell-cycle control (including B cell precursor cell cycle control).

In eukaryotes, aminopeptidases are associated with removal of the initiator methionine. This enzyme family is also involved in the metabolism of secreted regulatory molecules, such as hormones and neurotransmitters, and modulation of cell-cell interactions. In mammalian cells and tissues, these enzymes play a role in the terminal stages of protein degradation, and in cell-cycle control. Aminopeptidase also have a role in protein turnover and selective elimination of obsolete or defective proteins.

Industrial uses of this enzyme family include modification of amino termini in recombinantly expressed proteins. See A. Taylor (1993) TIBS 18: 1993:167-172.

Many aminopeptidases are metalloenzymes, requiring divalent cations for proteolytic activity. Most aminopeptidase metal binding sites coordinate Zn²⁺ or Co²⁺. However, the metal binding sites of certain aminopeptidases can readily bind Mn²⁺ and Mg²⁺. Sites involved in Zn²⁺ coordination include the “His His Glu” and “Asp Glu Lys” motifs.

Several aminopeptidase inhibitors have been identified. These inhibitors include bestatin (which has been shown to bind to the aminopeptidase active site), boronic and phosphonic acids, α-methylleucine and isoamylthioamide. See A. Taylor (1993) TIBS 18: 1993:167-172; Burley et al. (1992) J. Mol. Biol. 224:113-140; Taylor et al. (1993) Biochemistry 32:784-790.

Aminopeptidases play a role in the pathogenesis of a number of disorders including hypertension, cancer, cataracts, and leukemia, and inhibitors of these enzymes are currently being evaluated as potential therapeutics for many of these disorders. Aminopeptidase activity is also believed to contribute to the aging process. Accordingly, aminopeptidases are a major target for drug action and development. Therefore, it is valuable to the field of pharmaceutical development to identify and characterize previously unknown aminopeptidases.

Metallopeptidases. Proteases function in carcinogenesis by inactivating or activating regulators of the cell cycle, differentiation, programmed cell death, or other processes affecting cancer development and/or progression. Consistent with the model involving protease activity and tumor progression, certain protease inhibitors have been shown to be effective inhibitors of carcinogenesis both in vitro and in vivo.

Metallopeptidases are a group of widely distributed proteases that depend on bound Ca²⁺ or Zn²⁺ for activity; however, certain metallopeptidases can readily utilize Mn²⁺ and Mg²⁺. The biological functions of metallopeptidases include protein maturation and protein degradation, such as the degradation of extracellular matrix proteins. As such, metallopeptidases have been shown to have a role in tumor growth, metastasis, and angiogenesis.

Accordingly, metallopeptidases are a major target for drug action and development. Therefore, it is valuable to the field of pharmaceutical development to identify and characterize previously unknown metallopeptidases. The present invention advances the state of the art by providing a previously unidentified human metallopeptidase.

ADP-ribosyltransferases. Mono (ADP-ribosyl) transferase (EC 2.4.2.31) catalyzes the transfer of the ADP-ribose moiety of nicotinamide adenine dinucleotide (NAD) to an acceptor amino acid in proteins. In vertebrates, there is a family of such enzymes that transfer an NAD group to arginine. At least five distinct forms of this enzyme have been identified so far. Some of the forms are attached to the membrane by a GPI anchor while others seem to be secreted. These proteins are typically about 250 to 300 amino acid residues.

Mono-ADP ribosylation is a post-translational modification of proteins in which the ADP-ribose moiety of NAD is transferred to an acceptor protein and is responsible for the toxicity of some bacterial toxins, e.g,. cholera toxin and pertussis toxin. ADP-ribosyltransferase activity has been detected in viruses, bacteria, and eukaryotic cells. For example, cholera toxin ADP-ribosylates an arginine in the α-subunit of the stimulatory heterotrimeric guanine nucleotide-binding (G) protein, resulting in the activation of adenylyl cyclase and an increase in intracellular cyclic AMP. Eukaryotic ADP-ribosyltransferase activity has been detected in several tissues, and cDNAs have been cloned from rabbit, human skeletal muscle, chicken polymorphonuclear granulocytes, and nucleoblasts, and mouse lymphoma cells. Studies have shown that when the transferase cDNAs are transfected into mammalian cells, the skeletal muscle and mouse lymphocyte enzymes are extracellular glycosylphosphatidylinositol (GPI)-anchored proteins. Consistent with its extracellular location, the GPI-linked muscle transferase ADP-ribosylates integrin α-7 on cultured myotubes (Zolkiewska et al. (1993) J. Biol. Chem. 268:25273-25276). Also, inhibitor studies suggest that the muscle transferase may participate in the regulation of myogenesis (Kharadia, S.V. et al. (1992) Exp. Cell Res. 201:33-42). The muscle and lymphocyte ADP-ribosyltransferases catalyze the ADP-ribosylation of arginine, agmatine, and other simple guanidino compounds (Zolkiewska,A. et al. (1992) Proc. Natl. Acad. Sci. U.S.A. 89:11352-11356).

### SUMMARY OF THE INVENTION

The present invention is based, in part, on the discovery of novel human RING finger proteins, referred to herein as “8035 and 84242”. The nucleotide sequences of the cDNA's encoding 8035 and 84242 are shown in SEQ ID NO: 1 and SEQ ID NO: 5, respectively, and the amino acid sequences of the 8035 and 84242 polypeptides are shown in SEQ ID NO: 2 and SEQ ID NO: 6, respectively. In addition, the nucleotide sequences of the coding regions of these cDNA's are depicted in SEQ ID NO: 3 and SEQ ID NO: 7, respectively.

Accordingly, in one aspect the invention features nucleic acid molecules that encode the 8035 and 84242 proteins or polypeptides, e.g., biologically active portions of the 8035 and 84242 proteins. In a preferred embodiment, the isolated nucleic acid molecules encode polypeptides having the amino acid sequence of SEQ ID NO: 2 or SEQ ID NO: 6. In other embodiments, the invention provides isolated 8035 and 84242 nucleic acid molecules having the nucleotide sequences shown in SEQ ID NO: 1, SEQ ID NO: 3, SEQ ID NO: 5, SEQ ID NO: 7, or the sequences of the DNA inserts of the plasmids deposited with ATCC Accession Number ______ and ______. In still other embodiments, the invention provides nucleic acid molecules that are substantially identical (e.g., naturally occurring allelic variants) to the nucleotide sequences shown in SEQ ID NO: 1, SEQ ID NO: 3, SEQ ID NO: 5, SEQ ID NO: 7, or the sequences of the DNA inserts of the plasmids deposited with ATCC Accession Number ______ and ______. In other embodiments, the invention provides nucleic acid molecules that hybridize under stringent hybridization conditions to a nucleic acid molecule comprising the nucleotide sequence of SEQ ID NO: 1, SEQ ID NO: 3, SEQ ID NO: 5, SEQ ID NO: 7, or the sequences of the DNA inserts of the plasmids deposited with ATCC Accession Number ______ and ______, wherein the nucleic acid encodes a full length 8035 or 84242 protein or an active fragment thereof.

In a related aspect, the invention further provides nucleic acid constructs that include a 8035 or 84242 nucleic acid molecule described herein. In certain embodiments, the nucleic acid molecules of the invention are operatively linked to native or heterologous regulatory sequences. Also included, are vectors and host cells containing the 8035 and 84242 nucleic acid molecules of the invention e.g., vectors and host cells suitable for producing 8035 and 84242 nucleic acid molecules and polypeptides.

In another related aspect, the invention provides nucleic acid fragments suitable as primers or hybridization probes for the detection of 8035 and 84242-encoding nucleic acids.

In still another related aspect, isolated nucleic acid molecules that are antisense to an 8035 or an 84242 encoding nucleic acid molecule are provided.

In another aspect, the invention features 8035 and 84242 polypeptides, and biologically active or antigenic fragments thereof that are useful, e.g., as reagents or targets in assays applicable to treatment and diagnosis of 8035 and 84242-mediated or -related disorders. In another embodiment, the invention provides 8035 and 84242 polypeptides having 8035 or 84242 activity, respectively. Preferred polypeptides are 8035 proteins including at least one RING finger protein domain (C3HC4 type) and, preferably, having an 8035 activity, e.g., an 8035 activity as described herein; and 84242 proteins including at least one IBR (In Between RING Finger) domain and, preferably, having an 84242 activity, e.g., an 84242 activity as described herein.

In other embodiments, the invention provides 8035 and 84242 polypeptides, e.g., an 8035 or 84242 polypeptide having the amino acid sequence shown in SEQ ID NO: 2 or SEQ ID NO: 6, respectively; the amino acid sequences encoded by the cDNA inserts of the plasmids deposited with ATCC Accession Number ______ or ______; an amino acid sequence that is substantially identical to the amino acid sequence shown in SEQ ID NO: 2 or SEQ ID NO: 6; or an amino acid sequence encoded by a nucleic acid molecule having a nucleotide sequence that hybridizes under stringent hybridization conditions to a nucleic acid molecule comprising the nucleotide sequence of SEQ ID NO: 1, SEQ ID NO: 3, SEQ ID NO: 5, SEQ ID NO: 7, or the sequence of the DNA inserts of the plasmids deposited with ATCC Accession Number ______ or ______, wherein the nucleic acids encode a full length 8035 or 84242 protein or an active fragment thereof.

In a related aspect, the invention further provides nucleic acid constructs that include an 8035 or 84242 nucleic acid molecule described herein.

In a related aspect, the invention provides 8035 and 84242 polypeptides or fragments operatively linked to non-8035 and non-84242 polypeptides to form fusion proteins.

In another aspect, the invention features antibodies and antigen-binding fragments thereof, that react with, or more preferably specifically bind 8035 or 84242 polypeptides.

In another aspect, the invention provides methods of screening for compounds that modulate the expression or activity of the 8035 and 84242 polypeptides or nucleic acids.

In still another aspect, the invention provides a process for modulating 8035 and 84242 polypeptide or nucleic acid expression or activity, e.g. using the screened compounds. In certain embodiments, the methods involve treatment of conditions related to aberrant activity or expression of the 8035 and 84242 polypeptides or nucleic acids, such as conditions involving aberrant regulation of cellular proliferation and/or differentiation.

The invention also provides assays for determining the activity of or the presence or absence of 8035 and 84242 polypeptides or nucleic acid molecules in a biological sample, including for disease diagnosis.

In further aspect the invention provides assays for determining the presence or absence of a genetic alteration in a 8035 or 84242 polypeptide or nucleic acid molecule, including for disease diagnosis.

The present invention is also based, in part, on the discovery of a novel human aminopeptidase, referred to herein as “55304”. The nucleotide sequence of a cDNA encoding 55304 is shown in SEQ ID NO: 9, and the amino acid sequence of a 55304 polypeptide is shown in SEQ ID NO: 10. In addition, the nucleotide sequence of the coding region is depicted in SEQ ID NO: 11.

Accordingly, in one aspect the invention features a nucleic acid molecule which encodes a 55304 protein or polypeptide, e.g., a biologically active portion of the 55304 protein. In a preferred embodiment, the isolated nucleic acid molecule encodes a polypeptide having the amino acid sequence of SEQ ID NO: 10. In other embodiments, the invention provides an isolated 55304 nucleic acid molecule having the nucleotide sequence shown in SEQ ID NO: 9, SEQ ID NO: 11, or the sequence of the DNA insert of the plasmid deposited with ATCC Accession Number ______. In still other embodiments, the invention provides nucleic acid molecules that are substantially identical (e.g., naturally occurring allelic variants) to the nucleotide sequence shown in SEQ ID NO: 9, SEQ ID NO: 11, or the sequence of the DNA insert of the plasmid deposited with ATCC Accession Number ______. In other embodiments, the invention provides a nucleic acid molecule which hybridizes under stringent hybridization conditions to a nucleic acid molecule comprising the nucleotide sequence of SEQ ID NO: 9, SEQ ID NO: 11, or the sequence of the DNA insert of the plasmid deposited with ATCC Accession Number ______, wherein the nucleic acid encodes a full length 55304 protein or an active fragment thereof.

In a related aspect, the invention further provides nucleic acid constructs which include a 55304 nucleic acid molecule described herein. In certain embodiments, the nucleic acid molecules of the invention are operatively linked to native or heterologous regulatory sequences. Also included, are vectors and host cells containing the 55304 nucleic acid molecules of the invention e.g., vectors and host cells suitable for producing 55304 nucleic acid molecules and polypeptides.

In another related aspect, the invention provides nucleic acid fragments suitable as primers or hybridization probes for the detection of 55304-encoding nucleic acids.

In still another related aspect, isolated nucleic acid molecules that are antisense to a 55304 encoding nucleic acid molecule are provided.

In another aspect, the invention features 55304 polypeptides, and biologically active or antigenic fragments thereof that are useful, e.g., as reagents or targets in assays applicable to treatment and diagnosis of 55304-mediated or -related disorders. In another embodiment, the invention provides 55304 polypeptides having a 55304 activity. Preferred polypeptides are 55304 proteins including at least one aminopeptidase domain, and, preferably, having a 55304 activity, e.g., a 55304 activity as described herein.

In other embodiments, the invention provides 55304 polypeptides, e.g., a 55304 polypeptide having the amino acid sequence shown in SEQ ID NO: 10; the amino acid sequence encoded by the cDNA insert of the plasmid deposited with ATCC Accession Number ______; an amino acid sequence that is substantially identical to the amino acid sequence shown in SEQ ID NO: 10; or an amino acid sequence encoded by a nucleic acid molecule having a nucleotide sequence which hybridizes under stringent hybridization conditions to a nucleic acid molecule comprising the nucleotide sequence of SEQ ID NO: 9, SEQ ID NO: 11, or the sequence of the DNA insert of the plasmid deposited with ATCC Accession Number ______, wherein the nucleic acid encodes a full length 55304 protein or an active fragment thereof.

In a related aspect, the invention further provides nucleic acid constructs which include a 55304 nucleic acid molecule described herein.

In a related aspect, the invention provides 55304 polypeptides or fragments operatively linked to non-55304 polypeptides to form fusion proteins.

In another aspect, the invention features antibodies and antigen-binding fragments thereof, that react with, or more preferably specifically bind 55304 polypeptides.

In another aspect, the invention provides methods of screening for compounds that modulate the expression or activity of the 55304 polypeptides or nucleic acids.

In still another aspect, the invention provides a process for modulating 55304 polypeptide or nucleic acid expression or activity, e.g. using the screened compounds. In certain embodiments, the methods involve treatment of conditions related to aberrant activity or expression of the 55304 polypeptides or nucleic acids, such as conditions involving aberrant or deficient cellular proliferation or differentiation.

The invention also provides assays for determining the activity of or the presence or absence of 55304 polypeptides or nucleic acid molecules in a biological sample, including for disease diagnosis.

In further aspect the invention provides assays for determining the presence or absence of a genetic alteration in a 55304 polypeptide or nucleic acid molecule, including for disease diagnosis.

The present invention is based, in part, on the discovery of a novel human metallopeptidase, referred to herein as “52999”. The nucleotide sequence of a cDNA encoding 52999 is shown in SEQ ID NO: 12, and the amino acid sequence of a 52999 polypeptide is shown in SEQ ID NO: 13. In addition, the nucleotide sequence of the coding region is depicted in SEQ ID NO: 14.

Accordingly, in one aspect the invention features a nucleic acid molecule which encodes a 52999 protein or polypeptide, e.g., a biologically active portion of the 52999 protein. In a preferred embodiment, the isolated nucleic acid molecule encodes a polypeptide having the amino acid sequence of SEQ ID NO: 13. In other embodiments, the invention provides an isolated 52999 nucleic acid molecule having the nucleotide sequence shown in SEQ ID NO: 12, SEQ ID NO: 14, or the sequence of the DNA insert of the plasmid deposited with ATCC Accession Number ______. In still other embodiments, the invention provides nucleic acid molecules that are substantially identical (e.g., naturally occurring allelic variants) to the nucleotide sequence shown in SEQ ID NO: 12, SEQ ID NO: 14, or the sequence of the DNA insert of the plasmid deposited with ATCC Accession Number ______. In other embodiments, the invention provides a nucleic acid molecule which hybridizes under stringent hybridization conditions to a nucleic acid molecule comprising the nucleotide sequence of SEQ ID NO: 12, SEQ ID NO: 14, or the sequence of the DNA insert of the plasmid deposited with ATCC Accession Number ______, wherein the nucleic acid encodes a full length 52999 protein or an active fragment thereof.

In a related aspect, the invention further provides nucleic acid constructs which include a 52999 nucleic acid molecule described herein. In certain embodiments, the nucleic acid molecules of the invention are operatively linked to native or heterologous regulatory sequences. Also included, are vectors and host cells containing the 52999 nucleic acid molecules of the invention e.g., vectors and host cells suitable for producing 52999 nucleic acid molecules and polypeptides.

In another related aspect, the invention provides nucleic acid fragments suitable as primers or hybridization probes for the detection of 52999-encoding nucleic acids.

In still another related aspect, isolated nucleic acid molecules that are antisense to a 52999 encoding nucleic acid molecule are provided.

In another aspect, the invention features 52999 polypeptides, and biologically active or antigenic fragments thereof that are useful, e.g., as reagents or targets in assays applicable to treatment and diagnosis of 52999-mediated or -related disorders. In another embodiment, the invention provides 52999 polypeptides having a 52999 activity. Preferred polypeptides are 52999 proteins including at least one metallopeptidase domain, and, preferably, having a 52999 activity, e.g., a 52999 activity as described herein.

In other embodiments, the invention provides 52999 polypeptides, e.g., a 52999 polypeptide having the amino acid sequence shown in SEQ ID NO: 13; the amino acid sequence encoded by the cDNA insert of the plasmid deposited with ATCC Accession Number ______; an amino acid sequence that is substantially identical to the amino acid sequence shown in SEQ ID NO: 13; or an amino acid sequence encoded by a nucleic acid molecule having a nucleotide sequence which hybridizes under stringent hybridization conditions to a nucleic acid molecule comprising the nucleotide sequence of SEQ ID NO: 12, SEQ ID NO: 14, or the sequence of the DNA insert of the plasmid deposited with ATCC Accession Number ______, wherein the nucleic acid encodes a full length 52999 protein or an active fragment thereof.

In a related aspect, the invention further provides nucleic acid constructs which include a 52999 nucleic acid molecule described herein.

In a related aspect, the invention provides 52999 polypeptides or fragments operatively linked to non-52999 polypeptides to form fusion proteins.

In another aspect, the invention features antibodies and antigen-binding fragments thereof, that react with, or more preferably specifically bind 52999 polypeptides.

In another aspect, the invention provides methods of screening for compounds that modulate the expression or activity of the 52999 polypeptides or nucleic acids.

In still another aspect, the invention provides a process for modulating 52999 polypeptide or nucleic acid expression or activity, e.g. using the screened compounds. In certain embodiments, the methods involve treatment of conditions related to aberrant activity or expression of the 52999 polypeptides or nucleic acids, such as inflammatory conditions and conditions involving aberrant or deficient cellular proliferation or differentiation.

The invention also provides assays for determining the activity of or the presence or absence of 52999 polypeptides or nucleic acid molecules in a biological sample, including for disease diagnosis.

In further aspect the invention provides assays for determining the presence or absence of a genetic alteration in a 52999 polypeptide or nucleic acid molecule, including for disease diagnosis.

The present invention is based, in part, on the discovery of a novel human ribosyltransferase referred to herein as mono-ADP ribosyltransferase. The nucleotide sequence of a cDNA encoding ADP-ribosyltransferase is shown in SEQ ID NO: 19, and the amino acid sequence of a ADP-ribosyltransferase polypeptide is shown in SEQ ID NO: 20.

Accordingly, in one aspect the invention features a nucleic acid molecule which encodes a ADP-ribosyltransferase protein or polypeptide, e.g., a biologically active portion of the ADP-ribosyltransferase protein. In a preferred embodiment, the isolated nucleic acid molecule encodes a polypeptide having the amino acid sequence of SEQ ID NO: 20. In other embodiments, the invention provides an isolated ADP-ribosyltransferase nucleic acid molecule having the nucleotide sequence shown in SEQ ID NO: 19, or the sequence of the DNA insert of the plasmid deposited with ATCC Accession Number ______. In still other embodiments, the invention provides nucleic acid molecules that are substantially identical (e.g., naturally occurring allelic variants) to the nucleotide sequence shown in SEQ ID NO: 19, or the sequence of the DNA insert of the plasmid deposited with ATCC Accession Number ______. In other embodiments, the invention provides a nucleic acid molecule which hybridizes under stringent hybridization conditions to a nucleic acid molecule comprising the nucleotide sequence of SEQ ID NO: 19, or the sequence of the DNA insert of the plasmid deposited with ATCC Accession Number ______, wherein the nucleic acid encodes a full length ADP-ribosyltransferase protein or an active fragment thereof.

In a related aspect, the invention further provides nucleic acid constructs which include a ADP-ribosyltransferase nucleic acid molecule described herein. In certain embodiments, the nucleic acid molecules of the invention are operatively linked to native or heterologous regulatory sequences. Also included, are vectors and host cells containing the ADP-ribosyltransferase nucleic acid molecules of the invention e.g., vectors and host cells suitable for producing ADP-ribosyltransferase nucleic acid molecules and polypeptides.

In another related aspect, the invention provides nucleic acid fragments suitable as primers or hybridization probes for the detection of ADP-ribosyltransferase-encoding nucleic acids.

In still another related aspect, isolated nucleic acid molecules that are antisense to a ADP-ribosyltransferase encoding nucleic acid molecule are provided.

In another aspect, the invention features ADP-ribosyltransferase polypeptides, and biologically active or antigenic fragments thereof that are useful, e.g., as reagents or targets in assays applicable to treatment and diagnosis of ADP-ribosyltransferase-mediated or -related disorders. Treatment is defined as the application or administration of a therapeutic agent to a patient, or application or administration of a therapeutic agent to an isolated tissue or cell line from a patient, who has a disease, symptom of disease or a predispoisition toward a disease, with the purpose to cure, heal, alleviate, relieve, alter, remedy, ameliorate, improve or affect the disease, the symptoms of disease or the predisposition toward a disease. A therapeutic agent includes, but is not limited to, small molecules, peptides, antibodies, ribozymes and antisense oligonucleotides.

In another embodiment, the invention provides ADP-ribosyltransferase polypeptides having a ADP-ribosyltransferase activity. Preferred polypeptides are ADP-ribosyltransferase proteins including at least one transferase domain, and, preferably, having a ADP-ribosyltransferase activity, e.g., a ADP-ribosyltransferase activity described herein.

In other embodiments, the invention provides ADP-ribosyltransferase polypeptides, e.g., a ADP-ribosyltransferase polypeptide having the amino acid sequence shown in SEQ ID NO: 20; the amino acid sequence encoded by the cDNA insert of the plasmid deposited with ATCC Accession Number ______; an amino acid sequence that is substantially identical to the amino acid sequence shown in SEQ ID NO: 20; or an amino acid sequence encoded by a nucleic acid molecule having a nucleotide sequence which hybridizes under stringent hybridization conditions to a nucleic acid molecule comprising the nucleotide sequence of SEQ ID NO: 19, or the sequence of the DNA insert of the plasmid deposited with ATCC Accession Number ______, wherein the nucleic acid encodes a full length ADP-ribosyltransferase protein or an active fragment thereof.

In a related aspect, the invention further provides nucleic acid constructs which include a ADP-ribosyltransferase nucleic acid molecule described herein.

In a related aspect, the invention provides ADP-ribosyltransferase polypeptides or fragments operatively linked to non-ADP-ribosyltransferase polypeptides to form fusion proteins.

In another aspect, the invention features antibodies and antigen-binding fragments thereof, that react with, or more preferably specifically bind ADP-ribosyltransferase polypeptides.

In another aspect, the invention provides methods of screening for compounds that modulate the expression or activity of the ADP-ribosyltransferase polypeptides or nucleic acids.

In still another aspect, the invention provides a process for modulating ADP-ribosyltransferase polypeptide or nucleic acid expression or activity, e.g. using the screened compounds. In certain embodiments, the methods involve treatment of conditions related to aberrant activity or expression of the ADP-ribosyltransferase polypeptides or nucleic acids, such as conditions involving aberrant or deficient cellular proliferation/differentiation or aberrant metabolic function.

The invention also provides assays for determining the activity of or the presence or absence of ADP-ribosyltransferase polypeptides or nucleic acid molecules in a biological sample, including for disease diagnosis.

In further aspect the invention provides assays for determining the presence or absence of a genetic alteration in a ADP-ribosyltransferase polypeptide or nucleic acid molecule, including for disease diagnosis.

### BRIEF DESCRIPTION OF THE DRAWINGS

FIG. 1A-B depicts a cDNA sequence (SEQ ID NO: 1) and predicted amino acid sequence (SEQ ID NO: 2) of human 8035. The methionine-initiated open reading frame of human 8035 (without the 5′ and 3′ untranslated regions) extends from nucleotide position 613 to position 1914 of SEQ ID NO: 1 (coding sequence shown in SEQ ID NO: 3).

FIG. 2A-B depicts a cDNA sequence (SEQ ID NO: 5) and predicted amino acid sequence (SEQ ID NO: 6) of human 84242. The methionine-initiated open reading frame of human 84242 (without the 5′ and 3′ untranslated regions) extends from nucleotide position 744 to position 1955 of SEQ ID NO: 5 (coding sequence shown in SEQ ID NO: 7).

FIG. 3 depicts a hydropathy plot of human 8035. Relative hydrophobic residues are shown above the dashed horizontal line, and relative hydrophilic residues are below the dashed horizontal line. The cysteine residues (cys) and N glycosylation site (Ngly) are indicated by short vertical lines just below the hydropathy trace. The numbers corresponding to the amino acid sequence (shown in SEQ ID NO: 2) of human 8035 are indicated. Polypeptides of the invention include fragments that include: all or a part of a hydrophobic sequence (a sequence above the dashed line); or all or part of a hydrophilic fragment (a sequence below the dashed line). Other fragments include a cysteine residue or an N-glycosylation site.

FIG. 4 depicts a hydropathy plot of human 84242. Relative hydrophobic residues are shown above the dashed horizontal line, and relative hydrophilic residues are below the dashed horizontal line. The cysteine residues (cys) and N glycosylation site (Ngly) are indicated by short vertical lines just below the hydropathy trace. The numbers corresponding to the amino acid sequence (shown in SEQ ID NO: 6) of human 84242 are indicated. Polypeptides of the invention include fragments that include: all or a part of a hydrophobic sequence (a sequence above the dashed line); or all or part of a hydrophilic fragment (a sequence below the dashed line). Other fragments include a cysteine residue or an N-glycosylation site.

FIG. 5 depicts an alignment of the RING finger protein domain (C3HC4 type) of human 8035 with a consensus amino acid sequence derived from a hidden Markov model. The upper sequence is the consensus amino acid sequence (SEQ ID NO: 4), while the lower amino acid sequence corresponds to amino acids 380 to 421 of SEQ ID NO: 2.

FIG. 6 depicts an alignment of the IBR (In Between RING Fingers) protein domain of human 84242 with a consensus amino acid sequence derived from a hidden Markov model. The upper sequence is the consensus amino acid sequence (SEQ ID NO: 8), while the lower amino acid sequence corresponds to amino acids 2 to 67 of SEQ ID NO: 6.

FIG. 7A-C depicts a cDNA sequence (SEQ ID NO: 9) and predicted amino acid sequence (SEQ ID NO: 10) of human 55304. The methionine-initiated open reading frame of human 55304 (without the 5′ and 3′ untranslated regions) extends from nucleotide position 803 to position 2845 of SEQ ID NO: 9 (coding sequence shown in SEQ ID NO: 11).

FIG. 8 depicts a hydropathy plot of human 55304. Relative hydrophobic residues are shown above the dashed horizontal line, and relative hydrophilic residues are below the dashed horizontal line. The cysteine residues (cys) and N glycosylation site (Ngly) are indicated by short vertical lines just below the hydropathy trace. The numbers corresponding to the amino acid sequence (shown in SEQ ID NO: 10) of human 55304 are indicated. Polypeptides of the invention include fragments which include: all or a part of a hydrophobic sequence (a sequence above the dashed line); or all or part of a hydrophilic fragment (a sequence below the dashed line). Other fragments include a cysteine residue or an N-glycosylation site.

FIG. 9A-B depicts a cDNA sequence (SEQ ID NO: 12) and predicted amino acid sequence (SEQ ID NO: 13) of human 52999. The methionine-initiated open reading frame of human 52999 (without the 5′ and 3′ untranslated regions) extends from nucleotide position 194 to position 2470 of SEQ ID NO: 12 (coding sequence shown in SEQ ID NO: 14).

FIG. 10 depicts a hydropathy plot of human 52999. Relative hydrophobic residues are shown above the dashed horizontal line, and relative hydrophilic residues are below the dashed horizontal line. The cysteine residues (cys) and N glycosylation site (Ngly) are indicated by short vertical lines just below the hydropathy trace. The numbers corresponding to the amino acid sequence (shown in SEQ ID NO: 13) of human 52999 are indicated. Polypeptides of the invention include fragments which include: all or a part of a hydrophobic sequence (a sequence above the dashed line); or all or part of a hydrophilic fragment (a sequence below the dashed line). Other fragments include a cysteine residue or an N-glycosylation site.

FIG. 11 depicts an alignment of portions of the metallopeptidase domain of human 52999 with consensus amino acid sequences derived from hidden Markov models. The upper sequences are the consensus amino acid sequences for the Peptidase_M8 family of zinc metallopeptidases and the lower amino acid sequences correspond to amino acids of human 52999. The upper sequence of domain 1 of 4 is SEQ ID NO: 15 and the lower amino acid sequence corresponds to amino acids 180 to 192 of SEQ ID NO: 13. The upper sequence of domain 2 of 4 is SEQ ID NO: 16 and the lower amino acid sequence corresponds to amino acids 230 to 290 of SEQ ID NO: 13. The upper sequence of domain 3 of 4 is SEQ ID NO: 17 and the lower amino acid sequence corresponds to amino acids 354 to 409 of SEQ ID NO: 13. The upper sequence of domain 4 of 4 is SEQ ID NO: 18 and the lower amino acid sequence corresponds to amino acids 520 to 554 of SEQ ID NO: 13.

FIG. 12 depicts a cDNA sequence (SEQ ID NO: 19) and predicted amino acid sequence (SEQ ID NO: 20) of human ADP-ribosyltransferase. The methionine-initiated open reading frame of human ADP-ribosyltransferase (without the 5′ and 3′ untranslated regions) extends from nucleotide position 255 to position 1133 of SEQ ID NO: 19 (coding sequence shown in SEQ ID NO: 21).

FIG. 13 depicts a hydropathy plot of human ADP-ribosyltransferase. Relative hydrophobic residues are shown above the dashed horizontal line, and relative hydrophilic residues are below the dashed horizontal line. The cysteine residues (cys) and N glycosylation site (Ngly) are indicated by short vertical lines just below the hydropathy trace. The numbers corresponding to the amino acid sequence (shown in SEQ ID NO: 20) of human ADP-ribosyltransferase are indicated. Polypeptides of the invention include fragments which include: all or a part of a hydrophobic sequence (a sequence above the dashed line); or all or part of a hydrophilic fragment (a sequence below the dashed line). Other fragments include a cysteine residue or as N-glycosylation site.

FIG. 14 depicts an alignment of the human ADP-ribosyltransferase polypeptiode with a consensus amino acid sequence derived from a hidden Markov model. The upper sequence is the consensus amino acid sequence (SEQ ID NO: 22), while the lower amino acid sequence corresponds to amino acid 3 to amino acid 271 of SEQ ID NO: 20.

Other features and advantages of the invention will be apparent from the following detailed description, and from the claims.

### DETAILED DESCRIPTION OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

Human 8035 and 84242

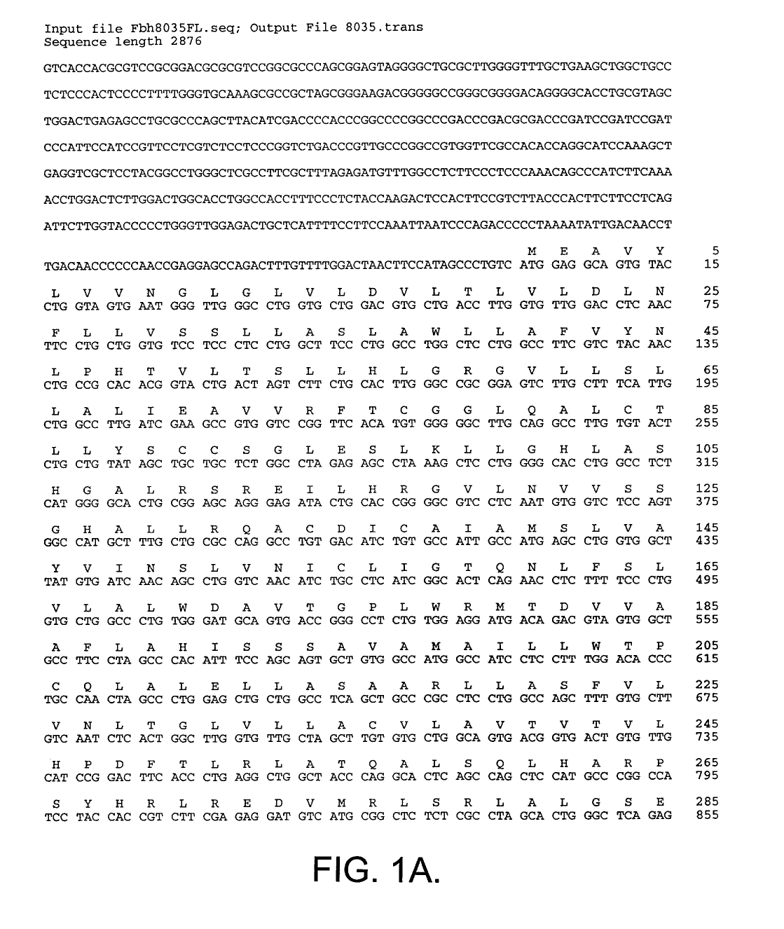

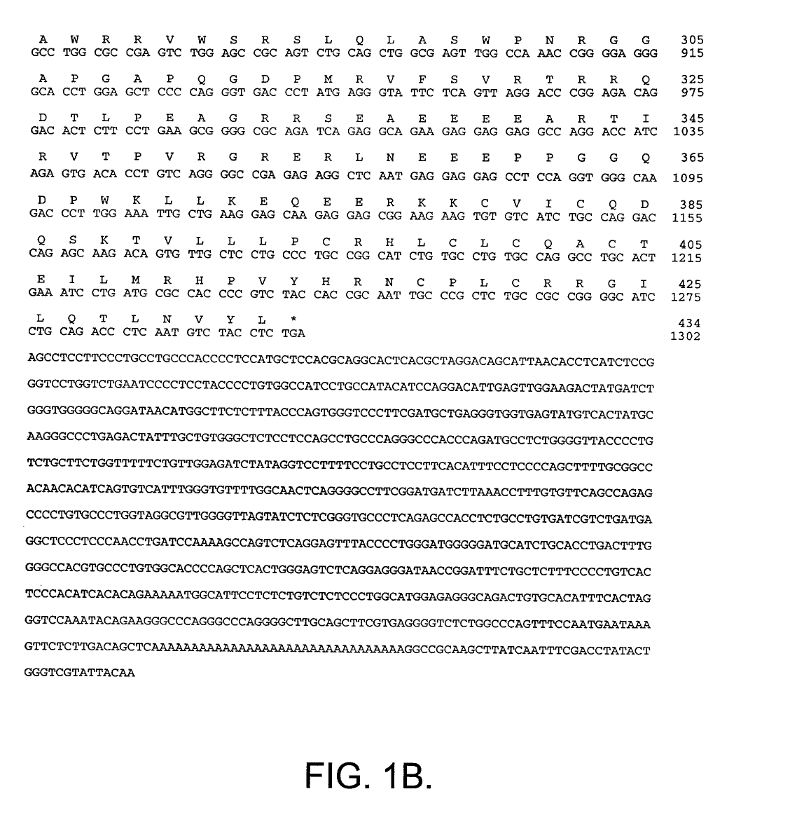

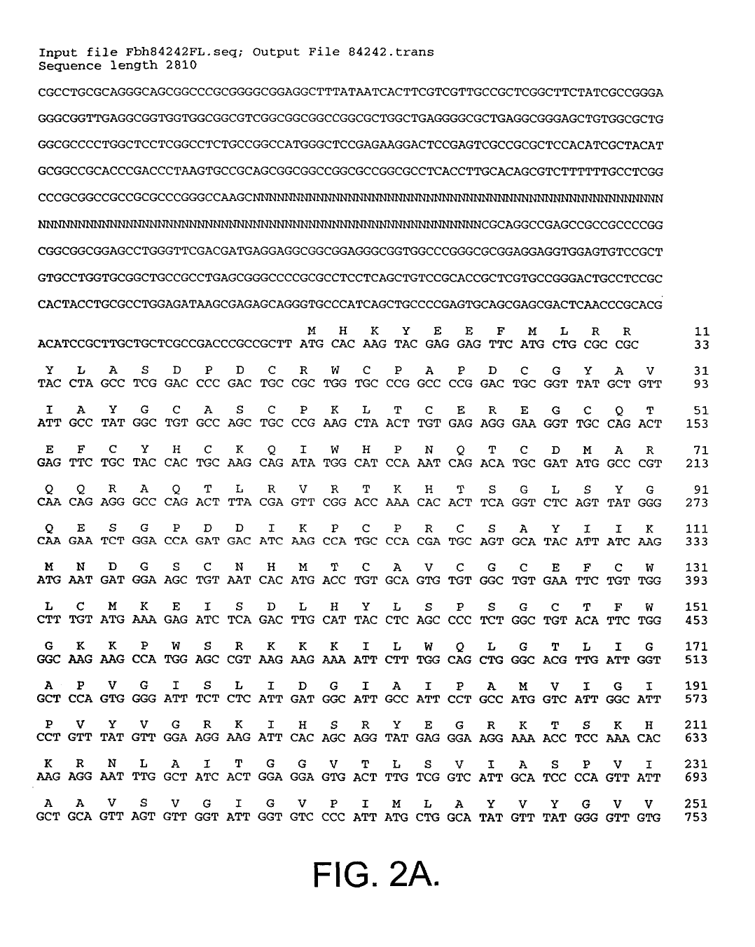

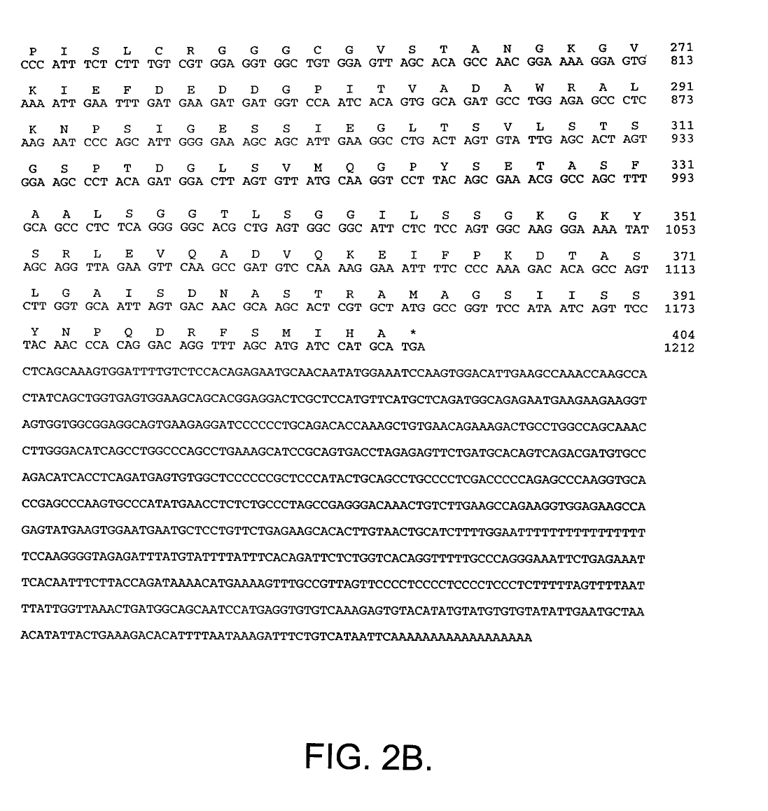

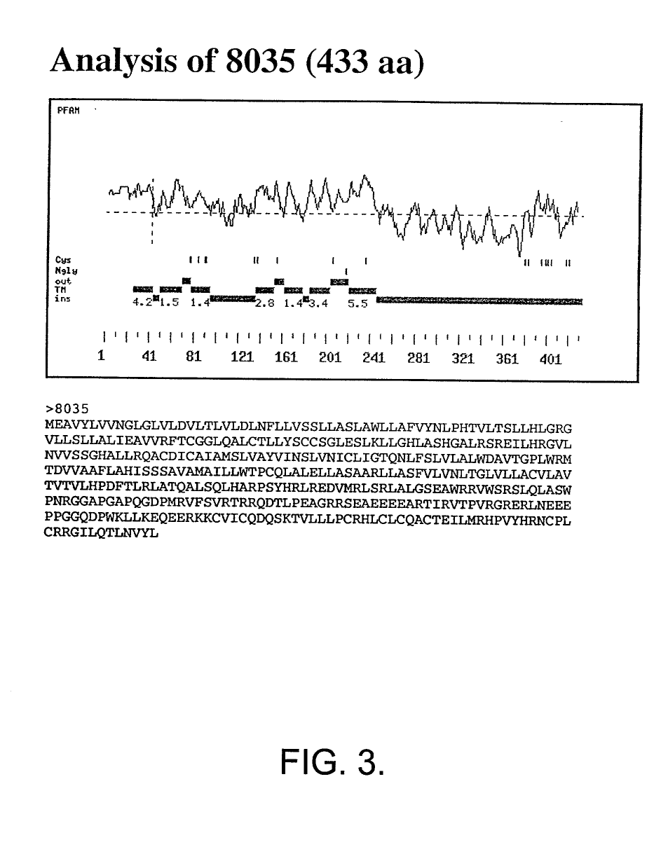

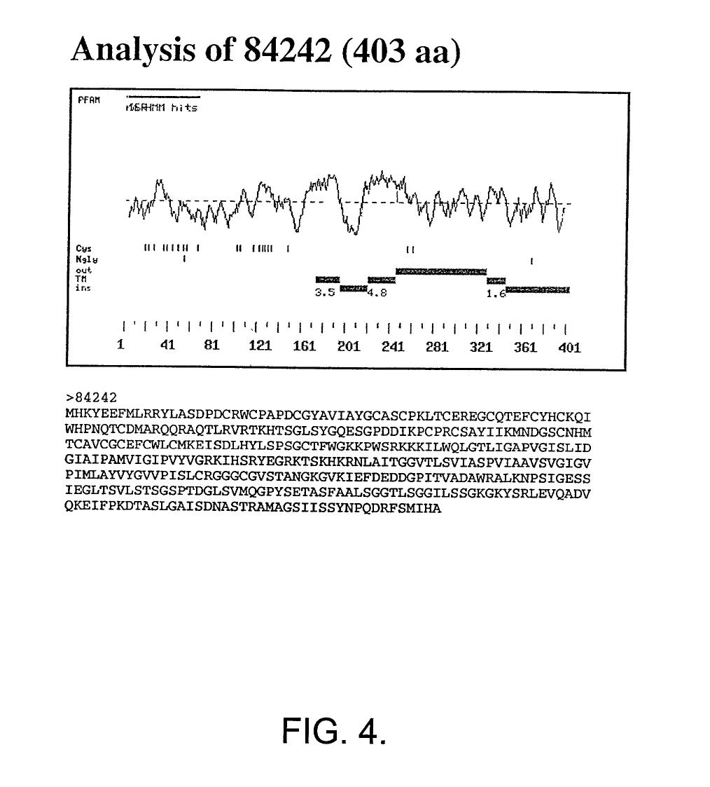

The human 8035 and 84242 sequences (FIGS. 1 and 2; SEQ ID NO: 1 and SEQ ID NO: 5, respectively), that are approximately 2876 and 2810 nucleotides long including untranslated regions, respectively, contain predicted methionine-initiated coding sequences of about 1302 and 1212 nucleotides (nucleotides 613-1914 of SEQ ID NO: 1; SEQ ID NO: 3, and nucleotides 744-1955 of SEQ ID NO: 5; SEQ ID NO: 7, respectively). The coding sequences encode a 433 and 403 amino acid protein (SEQ ID NO: 2 and SEQ ID NO: 6, respectively).

Human 8035 contains a predicted RING finger protein domain (C3HC4 type) (PFAM Accession PF00097) located at about amino acid residues 380-421 of SEQ ID NO: 2; and potential transmembrane domains are recognized from about amino acid residue 26-43, 50-69, 78-94, 136-152, 162-178, 185-203, and 221-245 of SEQ ID NO: 2.

Human 84242 contains a predicted IBR (In Between RING Fingers) domain (PFAM Accession PF01485) located at about amino acid residues 2-67 of SEQ ID NO: 6; and potential transmembrane domains are recognized from about amino acid residue 174 -195, 221-245, and 329-345 of SEQ ID NO: 6.

For general information regarding PFAM identifiers, PS prefix and PF prefix domain identification numbers, refer to Sonnhammer et al. (1997) Protein 28:405-420 and www.psc.edu/general/software/packages/pfam/pfam.html.

Plasmids containing the nucleotide sequences encoding human 8035 and 84242 were deposited with American Type Culture Collection (ATCC), 10801 University Boulevard, Manassas, Va. 20110-2209, on and assigned Accession Number ______ and ______, respectively. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. §112.

As stated above the 8035 proteins share significant structural characteristics with members of the C3HCH type RING finger protein family and the 84242 protein contains another cysteine-rich domain termed, IBR (In Between RING Fingers). The IBR domain has a C6HC consensus pattern that defines this structure as the forth family member of the zinc-binding RING, LIM, and LAP/PHD fingers (van der Reijden et al. (1999) Protein Sci. 8:1557-1561). The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., rat or mouse proteins. Members of a family can also have common functional characteristics.

A number of eukaryotic and viral proteins contain a C3HCH type RING finger domain. This conserved cysteine-rich RING domain binds two atoms of zinc, and is likely involved in mediating protein-protein interactions. The 3 dimensional structure of the zinc ligation system is unique to the RING domain and is referred to as the “cross-brace” motif.

As such, the 84242 polypeptides of the present invention can be expected to possess similar biological activities as the 8035 polypeptides of the invention and other RING finger protein family members.

Typically, RING finger family proteins play a role in diverse cellular processes. For example, proteins currently known to include the C3HC4 domain have a role in the mediation of such functions as recombination, particularly the rearrangement of immunoglobulin and T-cell receptor genes; the regulation of gene expression, particularly in various tumor cells, and as a trans-activator and/or -repressor of the expression of many viral and cellular promoters including the interleukin-2 receptor alpha chain; the maintenance of the segment-specific repression of homeotic selector genes and as a DNA-binding protein involved in X chromosome dosage compensation; developmental regulation, particularly male germ cell development and the regulation of photomorphogenesis; cellular differentiation, particularly differentiation of acute leukemia cells; the stabilization of protein-protein interactions, particularly the stabilization of the complex between the CDK7 kinase and cyclin H; peroxisome biogenesis, particularly in Zellweger syndrome, an autosomal recessive disorder associated with peroxisomal deficiencies; the postranscriptional regulation of genes, particularly in VSG expression sites; the regulation of adenylate cyclase activity; and the regulation of DNA repair.

Genetic mutations, recombinations and chromosomal translocations in RING finger protein family members have been implicated in diseases such as cancer, particularly mammalian breast and ovarian cancer, systemic lupus erythematosus, acute promyelocytic leukemia (APL), VHL disease, primary Sjogren's syndrome, Zellweger syndrome, and autosomal juvenile parkinsonism. In addition, RING finger protein family members have been shown to contribute to the pathogenesis of certain viral diseases including those caused by HSV and HIV.

Thus, the molecules of the present invention may be involved in one or more of: 1) regulation of recombination; 2) regulation of gene expression; 3) developmental regulation; 4) regulation of cellular proliferation and differentiation; 5) regulation of tumor cell growth; 6) stabilization of protein-protein interactions; 7) postranscriptional regulation of genes; 8) regulation of adenylate cyclase activity; 9) regulation of the cell cycle; 10) regulation of X chromosome dosage compensation; 11) regulation of DNA repair; 12) regulation of viral pathogenesis; 13) regulation of protein degradation from the ER; and 14) regulation of apoptosis.

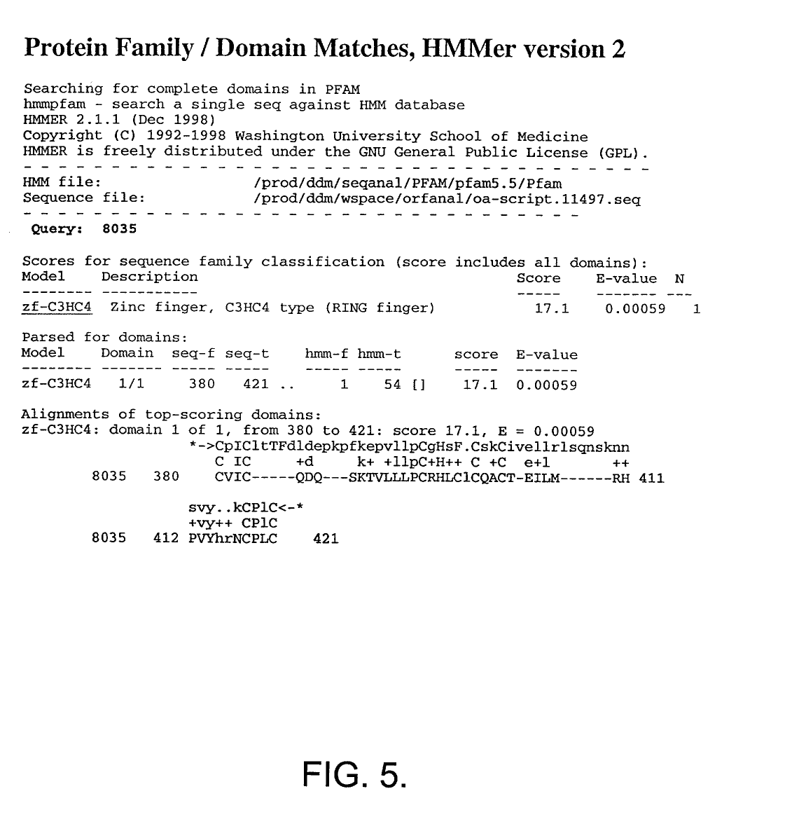

As used herein, the term “RING finger protein domain” includes an amino acid sequence of about 30-60 amino acid residues in length and having a bit score for the alignment of the sequence to the RING finger protein domain (HMM) of at least 8. Preferably, a RING finger protein domain has a bit score for the alignment of the sequence to the RING finger protein domain (HMM) of at least 16 or greater. The RING finger protein domain (HMM) has been assigned the PFAM Accession PF00097 (pfam.wustl.edu/). An alignment of the RING finger protein domain (amino acids 380-421 of SEQ ID NO: 2) of human 8035 with a consensus amino acid sequence derived from a hidden Markov model is depicted in FIG. 5.

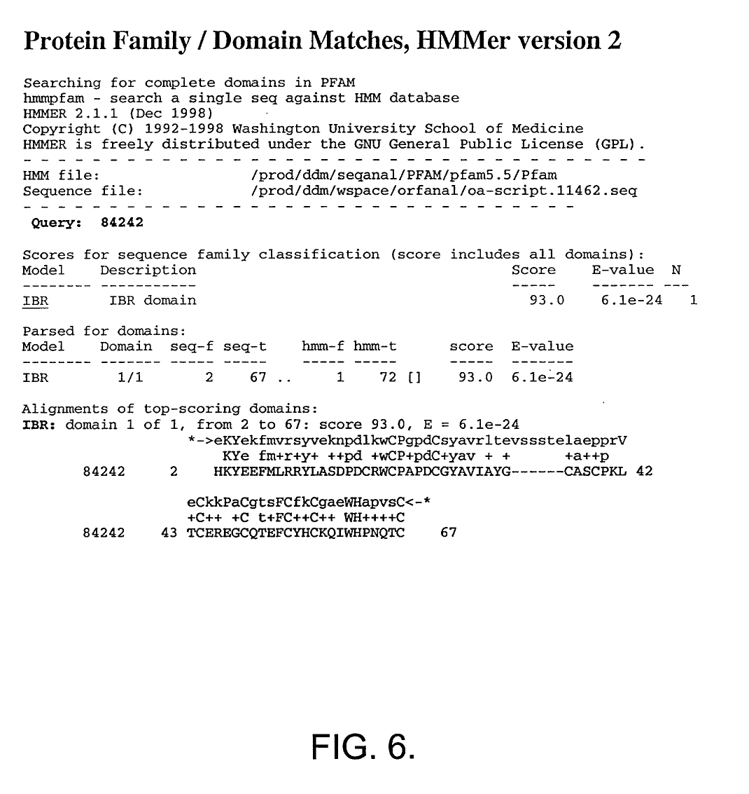

Herein, the term “RING finger protein family member” may also include a polypeptide that possess an IBR domain as described above. An IBR domain includes an amino acid sequence of about 45-70 amino acid residues in length and having a bit score for the alignment of the sequence to the IBR protein domain (HMM) of at least 8. Preferably, an IBR protein domain has a bit score for the alignment of the sequence to the IBR domain (HMM) of at least 16 or greater. The IBR domain (HMM) has been assigned the PFAM Accession PF01485 (pfam.wustl.edu/). An alignment of the IBR protein domain (amino acids 2-67 of SEQ ID NO: 6) of human 84242 with a consensus amino acid sequence derived from a hidden Markov model is depicted in FIG. 6.

In a preferred embodiment an 8035 polypeptide or protein has at least one RING finger domain or region that includes at least about 30-60 amino acid residues with at least about 60%, 70%, 80%, 90%, 95%, 99%, or 100% homology with a RING finger domain, e.g., the RING finger protein domains of human 8035 (e.g., amino acid residues 380-421 of SEQ ID NO: 2).

In another preferred embodiment an 84242 polypeptide or protein has at least one RING finger domain as described above as well as an IBR domain or region that includes at least about 45-70 amino acid residues with at least about 60%, 70%, 80%, 90%, 95%, 99%, or 100% homology with an IBR domain, e.g., the IBR protein domain of human 84242 (e.g., amino acid residues 2-67 of SEQ ID NO: 6).

To identify the presence of a RING finger domain and/or an IBR domain in a 8035 or 84242 protein sequence, and make the determination that a polypeptide or protein of interest has a particular profile, the amino acid sequence of the protein can be searched against a database of HMMs (e.g., the Pfam database, release 2.1) using the default parameters (www.sanger.ac.uk/Software/Pfam/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J Mol. Biol. 235:1501-1531; and Stultz et al. (1993) Protein Sci. 2:305-314, the contents of which are incorporated herein by reference.

As the 8035 and 84242 polypeptides of the invention may modulate 8035 and 84242-mediated activities, they may be useful for developing novel diagnostic and therapeutic agents for 8035 and 84242-mediated or related disorders, as described below.

As used herein, an “8035 or 84242 activity”, “biological activity of 8035 or 84242” or “functional activity of 8035 or 84242”, refers to an activity exerted by an 8035 or 84242 protein, polypeptide or nucleic acid molecule on e.g., an 8035 or 84242-responsive cell or on an 8035 or 84242 substrate, e.g., a protein substrate, as determined in vivo or in vitro. In one embodiment, an 8035 or 84242 activity is a direct activity, such as an association with an 8035 or 84242 target molecule. A 8035 or 84242 “target molecule” or “binding partner” or “ligand” or “substrate” is a molecule with which an 8035 or 84242 protein binds or interacts in nature, e.g., an E2 polypeptide or other protein substrate that an 8035 or 84242 protein binds to facilitate protein ubiquitination and protein degradation.

An 8035 or 84242 activity can also be an indirect activity, e.g., a cellular signaling activity mediated by interaction of the 8035 or 84242 protein with an 8035 or 84242 ligand. For example, the 8035 and 84242 proteins of the present invention can have one or more of the following activities: 1) regulation of recombination, including the rearrangement of immunoglobulin and T-cell receptor genes; 2) regulation of gene expression such as by the transactivation and/or repression of the expression of various promoters; 3) developmental regulation including regulation of male germ cell development, and maintenance of the segment-specific repression of homeotic selector genes; 4) regulation of cellular proliferation and differentiation; 5) regulation of tumor cell growth; 6) stabilization of protein-protein interactions such as stabilization of the complex between certain cyclin regulated kinases; 7) postranscriptional regulation of genes including VSG genes; 8) regulation of adenylate cyclase activity; 9) regulation of the cell cycle such as regulation of mitosis; 10) regulation of X chromosome dosage compensation; 11) regulation of DNA repair; 12) regulation of viral pathogenesis; 13) regulation of protein degradation from the ER; 14) regulation of photomorphogenesis; 15) regulation of peroxisome biogenesis; and 16) regulation of apoptosis.

Accordingly, 8035 and 84242 protein may be mediate various disorders, particularly cellular proliferative and/or differentiative disorders. Indeed, genetic mutations, recombinations and chromosomal translocations in RING finger protein family members have been implicated in diseases such as cancer, particularly mammalian breast and ovarian cancer; systemic lupus erythematosus; acute promyelocytic leukemia (APL); VHL disease; primary Sjogren's syndrome; Zellweger syndrome; and autosomal juvenile parkinsonism. In addition, RING finger protein family members have been shown to contribute to the pathogenesis of certain viral diseases including those caused by HSV and HIV.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

The 8035 and 84242 nucleic acid and protein of the invention can be used to treat and/or diagnose a variety of proliferative disorders. E.g., such disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit. Rev. in Oncol/Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicalla-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degeneration, multiple system atrophy, including striatonigral degeneration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B₁) deficiency and vitamin B₁₂ deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease.

Human 55304

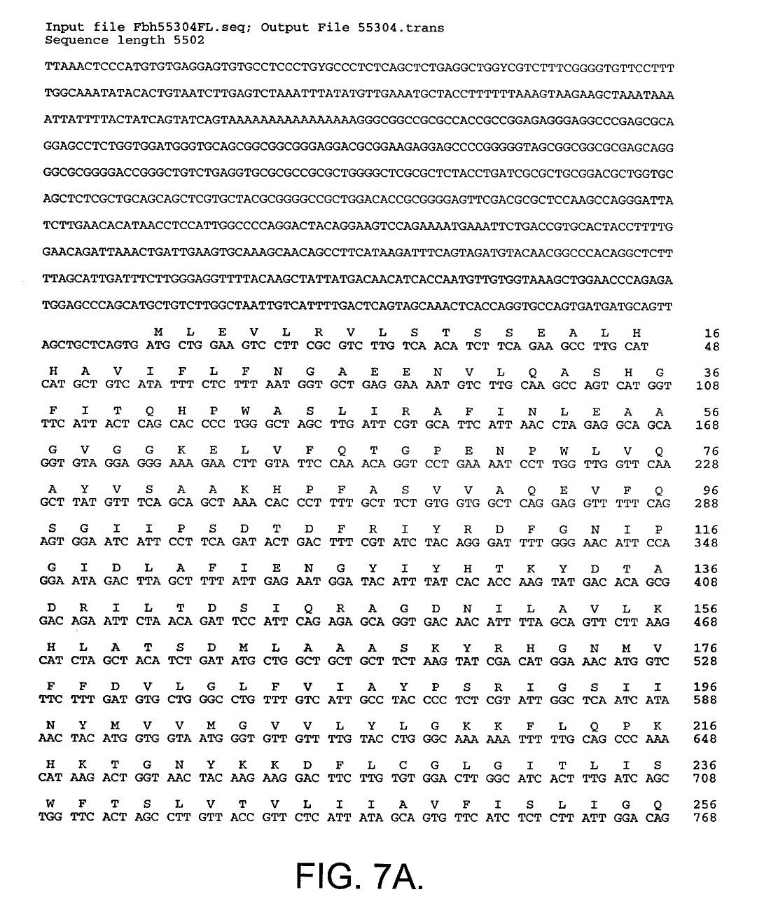

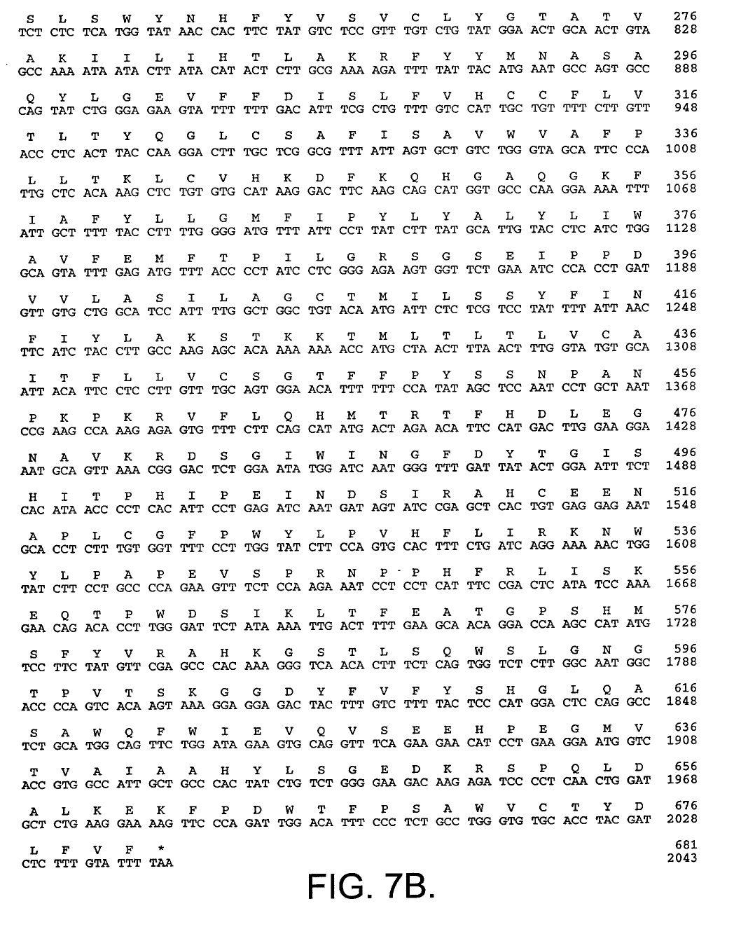

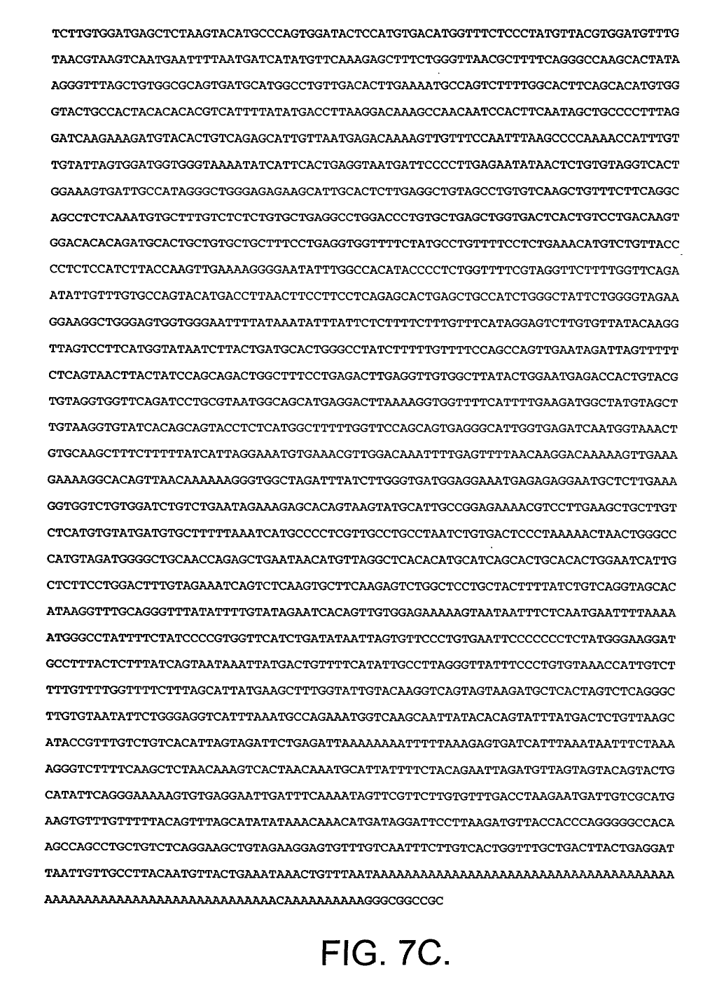

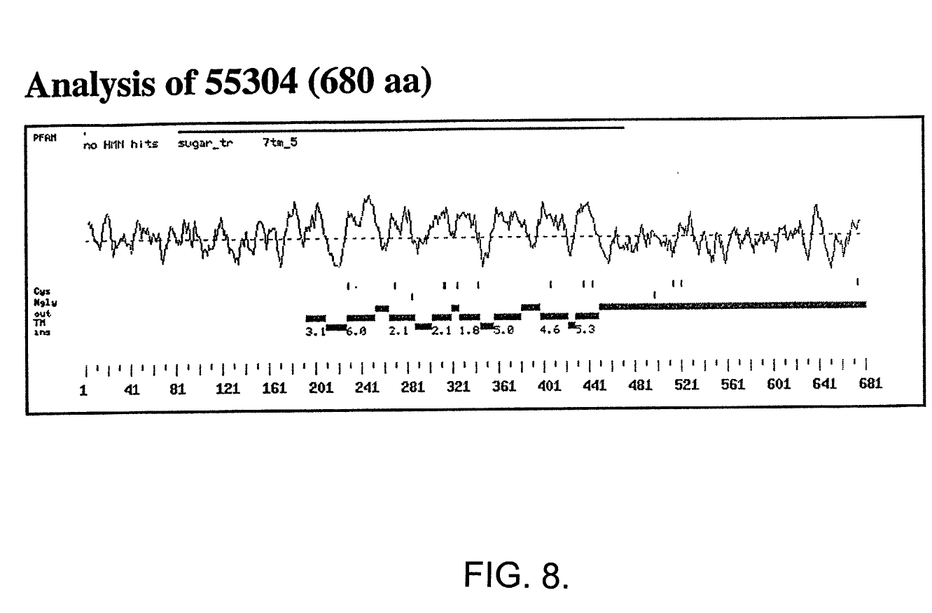

The human 55304 sequence (FIG. 7A-C; SEQ ID NO: 9), which is approximately 5502 nucleotides long including untranslated regions, contains a predicted methionine-initiated coding sequence of about 2043 nucleotides (nucleotides 803-2845 of SEQ ID NO: 9; SEQ ID NO: 11). The coding sequence encodes a 680 amino acid protein (SEQ ID NO: 10). Human 55304 shows a high degree of sequence conservation with known aminopeptidases at 16 key residues (amino acid numbers 318, 336, 344, 346, 363, 374, 397,399, 410,411,412,425,448, 450,489,508 of SEQ ID NO: 10). These amino acids are conserved between the 55340 polypeptide and the corresponding residues from a leucyl aminopeptidase from Vibrio proteolyticus (SwissProt Accession No. Q01693), an aminopeptidase from Streptomyces griseus (SwissProt Accession No. P80561), and aminopeptidase Y from Saccharomyces cerevisiae (SwissProt Accession No. P37302).

The 55304 polypeptide contains 8 putative transmembrane domains. These domains are located at amino acids 192-208, 227-251, 264-286, 302-318, 326-343, 356-379, 397-421, and 428-448 of the 55304 amino acid sequences shown in SEQ ID NO: 10.

The 55304 protein also contains the following ProDom domain matches: protein aminopeptidase/T1F15.12/HSP26-TIF32 hydrolase (amino acids 1-69 of SEQ ID NO: 10) and YBS_{—}4/HSP26-TIF32 hydrolase/aminopeptidase zinc metalloprotease (amino acids 83-206 of SEQ ID NO: 10).

A plasmid containing the nucleotide sequence encoding human 55304 was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. § 112.

The 55304 protein contains structural characteristics in common with members of the aminopeptidase family.

As used herein, the term “aminopeptidase” refers to a protein or polypeptide which is capable of catalyzing the removal of an amino acid from the amino terminus of a peptide substrate. Aminopeptidases can have a specificity for specific amino acids. For example, the removal of the amino-terminal methionine from proteins and peptides is catalyzed by the methionine aminopeptidase class of aminopeptidases.

As referred to herein, aminopeptidases preferably include a catalytic domain of about 100-250 amino acid residues in length, preferably about 130-210 amino acid residues in length, or more preferably about 180-200 amino acid residues in length. An aminopeptidase domain typically includes conserved residues (i.e. identical residues or conservatively substituted residues as defined elsewhere herein) in at least 8, at least 9, at least 10, at least 11, at least 12, at least 13, at least 14, at least 15, or at 16 sites in the amino acid sequence of the protein. These sites are located at amino acids 318, 336, 344, 346, 363, 374, 397, 399, 410, 411, 412, 425, 448, 450, 489, 508 of SEQ ID NO: 10.

Typically, aminopeptidases play a role in diverse cellular processes. For example, aminopeptidases function in protein maturation, in the terminal degradation of polypeptides, in hormone level regulation, in the regulation of the renin-angiotensin system, and in cell cycle control.

Thus, the molecules of the present invention may be involved in one or more of: 1) the removal of an amino acid from the amino terminus of a peptide substrate; 2) protein maturation; 3) the terminal degradation of proteins; 4) the modulation of hormone levels; 5) the regulation of the cell cycle; or 6) the regulation of the renin-angiotensin system.

In a preferred embodiment 55304 polypeptide or protein has an “aminopeptidase domain” or a region which includes at least about 100-250 more preferably about 130-200 or 160-200 amino acid residues and has at least about 60%, 70%, 80%, 90%, 95%, 99%, or 100% homology with an “aminopeptidase domain,” e.g., the aminopeptidase domain of human 55304 (e.g., amino acid residues 318-508, particularly amino acid residues 318, 336, 344, 346, 363, 374, 397, 399, 410, 411, 412, 425, 448, 450, 489, and 508 of SEQ ID NO: 10).

In one embodiment, a 55304 protein includes at least one transmembrane domain. As used herein, the term “transmembrane domain” includes an amino acid sequence of about 15 amino acid residues in length that spans a phospholipid membrane. More preferably, a transmembrane domain includes about at least 18, 20, 22, 24, 25, 30, 35 or 40 amino acid residues and spans a phospholipid membrane. Transmembrane domains are rich in hydrophobic residues, and typically have an α-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, pfam.wustl. edu/cgi-bin/getdesc?name=7tm-1, and Zagotta W. N. et al. (1996) Annual Rev. Neuronsci. 19:235-63, the contents of which are incorporated herein by reference.

In a preferred embodiment, a 55304 polypeptide or protein has at least one transmembrane domain or a region which includes at least 18, 20, 22, 24, 25, 30, 35 or 40 amino acid residues and has at least about 60%, 70% 80% 90% 95%, 99%, or 100% sequence identity with a “transmembrane domain,” e.g., at least one transmembrane domain of human 55304 (e.g., amino acid residues 192-208, 227-251, 264-286, 302-318, 326-343, 356-379, 397-421, or 428-448 of SEQ ID NO: 10).

In another embodiment, a 55304 protein includes at least one “non-transmembrane domain.” As used herein, “non-transmembrane domains” are domains that reside outside of the membrane. When referring to plasma membranes, non-transmembrane domains include extracellular domains (i.e., outside of the cell) and intracellular domains (i.e., within the cell). When referring to membrane-bound proteins found in intracellular organelles (e.g., mitochondria, endoplasmic reticulum, peroxisomes and microsomes), non-transmembrane domains include those domains of the protein that reside in the cytosol (i.e., the cytoplasm), the lumen of the organelle, or the matrix or the intermembrane space (the latter two relate specifically to mitochondria organelles). The C-terminal amino acid residue of a non-transmembrane domain is adjacent to an N-terminal amino acid residue of a transmembrane domain in a naturally-occurring 55304, or 55304-like protein.

In a preferred embodiment, a 55304 polypeptide or protein has a “non-transmembrane domain” or a region which includes at least about 1-250, preferably about 1-2311, more preferably about 5-231 amino acid residues, and has at least about 60%, 70% 80% 90% 95%, 99% or 100% sequence identity with a “non-transmembrane domain”, e.g., a non-transmembrane domain of human 55304 (e.g., residues 1-191, 209-226, 252-263, 287-301, 319-325, 344-355, 380-396, 422-427, or 449-680 of SEQ ID NO: 10). Preferably, a non-transmembrane domain is capable of catalytic activity (e.g., catalyzing the removal of an amino terminal amino acid from a peptide substrate).

A non-transmembrane domain located at the N-terminus of a 55304 protein or polypeptide is referred to herein as an “N-terminal non-transmembrane domain.” As used herein, an “N-terminal non-transmembrane domain” includes an amino acid sequence having about 1-350, preferably about 50-325, more preferably about 80-320, or even more preferably about 120-191 amino acid residues in length and is located outside the boundaries of a membrane. For example, an N-terminal non-transmembrane domain is located at about amino acid residues 1-191 of SEQ ID NO: 10.

Similarly, a non-transmembrane domain located at the C-terminus of a 55304 protein or polypeptide is referred to herein as a “C-terminal non-transmembrane domain.” As used herein, an “C-terminal non-transmembrane domain” includes an amino acid sequence having about 1-300, preferably about 15-290, preferably about 20-270, more preferably about 25-231 amino acid residues in length and is located outside the boundaries of a membrane. For example, a C-terminal non-transmembrane domain is located at about amino acid residues 680-449 of SEQ ID NO: 10.

As the 55304 polypeptides of the invention may modulate 55304-mediated activities, they may be useful as of for developing novel diagnostic and therapeutic agents for 55304-mediated or related disorders, as described below.

As used herein, a “55304 activity”, “biological activity of 55304” or “functional activity of 55304”, refers to an activity exerted by a 55304 protein, polypeptide or nucleic acid molecule on e.g., a 55304-responsive cell or on a 55304 substrate, e.g., a protein substrate, as determined in vivo or in vitro. In one embodiment, a 55304 activity is a direct activity, such as an association with a 55304 target molecule. A “target molecule” or is a molecule with which a 55304 protein binds or interacts in nature, e.g., a peptide substrate from which 55304 removes an amino acid. A 55304 activity can also be an indirect activity, e.g., a cellular signaling activity mediated by the product of 55304 proteolysis. For example, the 55304 proteins of the present invention can have one or more of the following activities: 1) the removal of an amino acid from the amino terminus of a peptide substrate 2) protein maturation 3) the terminal degradation of proteins; 4) the modulation of hormone levels; 5) the regulation of the cell cycle; or 6) the regulation of the renin-angiotensin system.

Accordingly, 55304 protein may be mediate various disorders, including cellular proliferative and/or differentiative disorders, hypertensive disorders, hormonal disorders, and disorders related to protein maturation and degradation.

The 55304 nucleic acid and protein of the invention can be used to treat and/or diagnose a variety of proliferative disorders (see above for examples of such disorders).

Disorders involving the heart, include but are not limited to, heart failure, including but not limited to, cardiac hypertrophy, left-sided heart failure, and right-sided heart failure; ischemic heart disease, including but not limited to angina pectoris, myocardial infarction, chronic ischemic heart disease, and sudden cardiac death; hypertensive heart disease, including but not limited to, systemic (left-sided) hypertensive heart disease and pulmonary (right-sided) hypertensive heart disease; valvular heart disease, including but not limited to, valvular degeneration caused by calcification, such as calcific aortic stenosis, calcification of a congenitally bicuspid aortic valve, and mitral annular calcification, and myxomatous degeneration of the mitral valve (mitral valve prolapse), rheumatic fever and rheumatic heart disease, infective endocarditis, and noninfected vegetations, such as nonbacterial thrombotic endocarditis and endocarditis of systemic lupus erythematosus (Libman-Sacks disease), carcinoid heart disease, and complications of artificial valves; myocardial disease, including but not limited to dilated cardiomyopathy, hypertrophic cardiomyopathy, restrictive cardiomyopathy, and myocarditis; pericardial disease, including but not limited to, pericardial effusion and hemopericardium and pericarditis, including acute pericarditis and healed pericarditis, and rheumatoid heart disease; neoplastic heart disease, including but not limited to, primary cardiac tumors, such as myxoma, lipoma, papillary fibroelastoma, rhabdomyoma, and sarcoma, and cardiac effects of noncardiac neoplasms; congenital heart disease, including but not limited to, left-to-right shunts—late cyanosis, such as atrial septal defect, ventricular septal defect, patent ductus arteriosus, and atrioventricular septal defect, right-to-left shunts—early cyanosis, such as tetralogy of fallot, transposition of great arteries, truncus arteriosus, tricuspid atresia, and total anomalous pulmonary venous connection, obstructive congenital anomalies, such as coarctation of aorta, pulmonary stenosis and atresia, and aortic stenosis and atresia, and disorders involving cardiac transplantation.

Human 52999

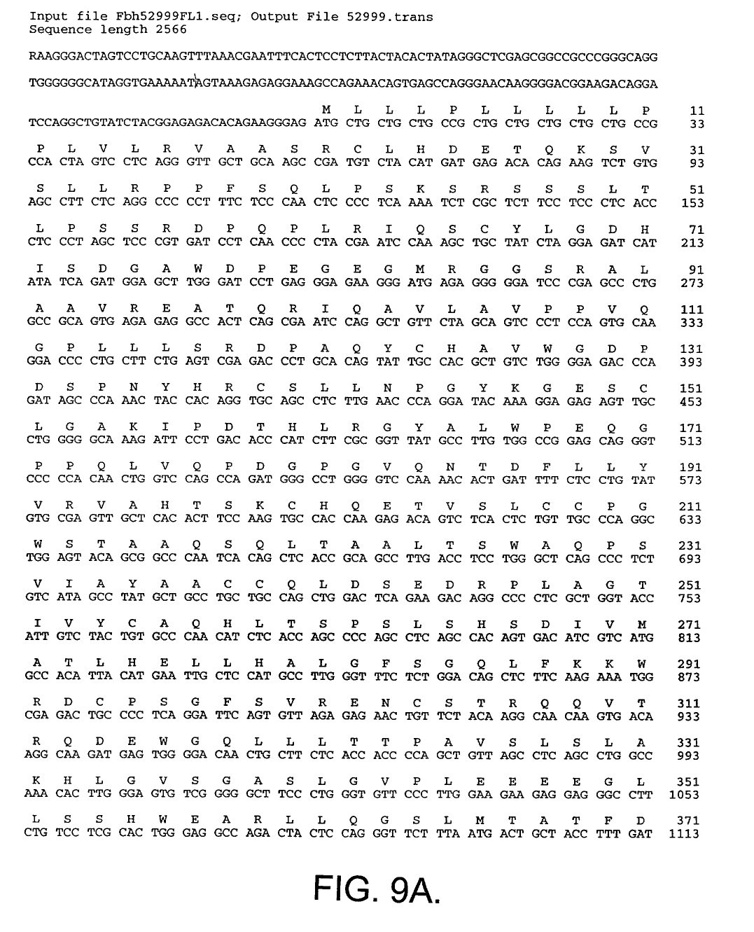

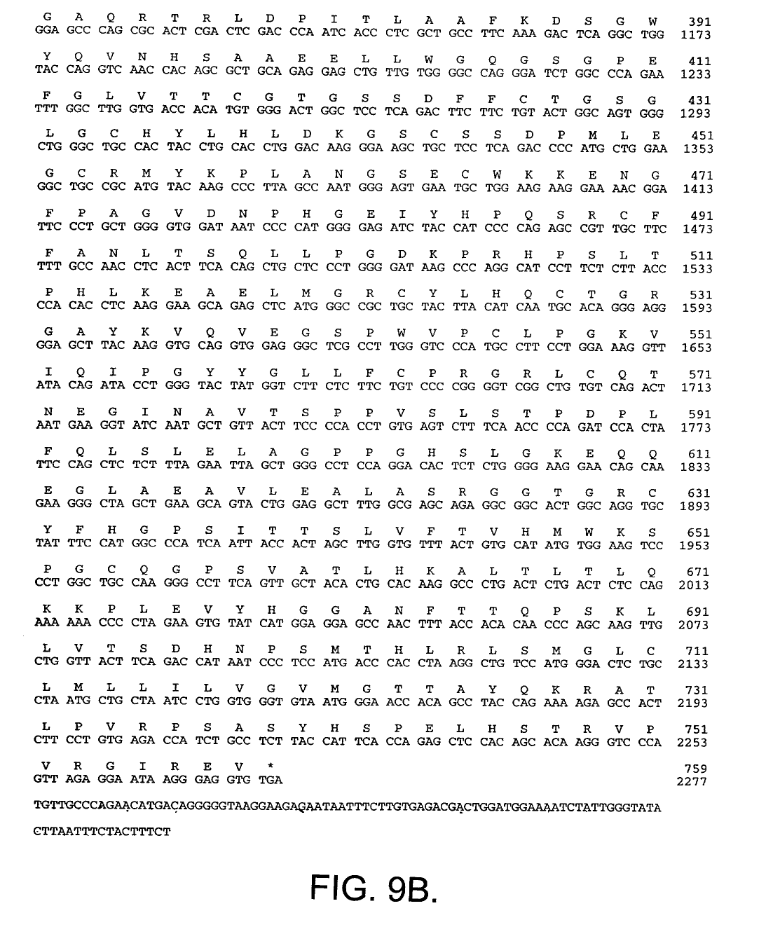

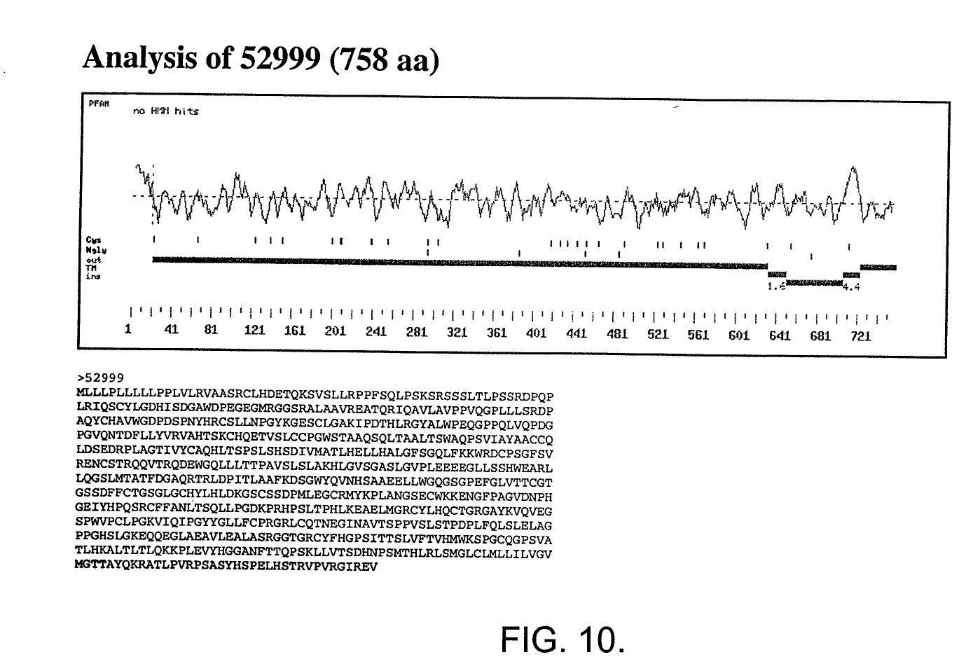

The present invention provides the human 52999 sequence (FIG. 9; SEQ ID NO: 12), which is approximately 2566 nucleotides long including untranslated regions, contains a predicted methionine-initiated coding sequence of about 2277 nucleotides (nucleotides 194 to 2470 of SEQ ID NO: 12; SEQ ID NO: 14). The coding sequence encodes a 758 amino acid protein (SEQ ID NO: 13).

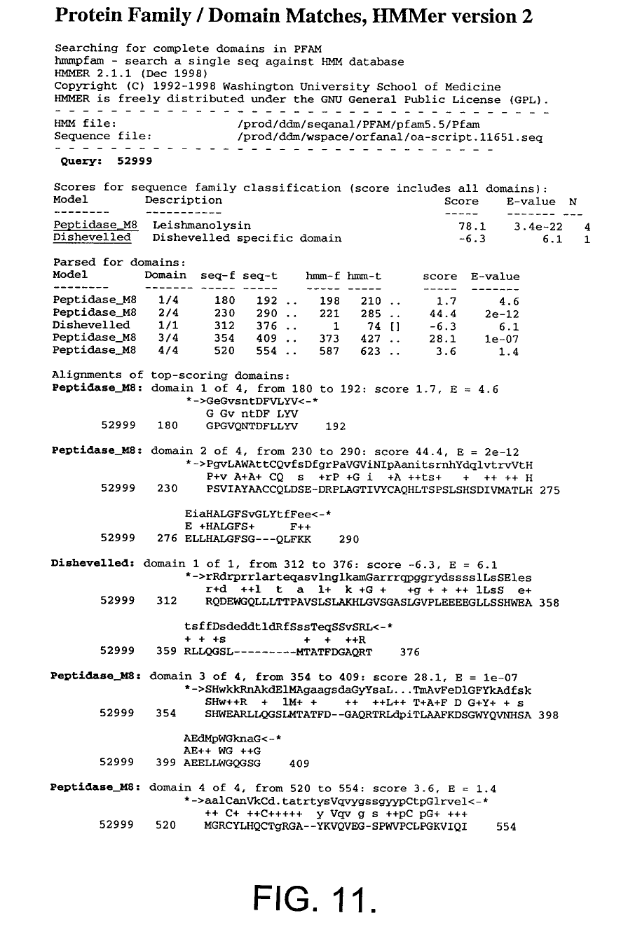

The protein form of 52999 after cleavage of the predicted signal sequence is approximately 739 amino acid residues in length (from about amino acid 20 to amino acid 758 of SEQ ID NO: 13). Human 52999 (SEQ ID NO: 13) contains regions of high homology located at about amino acid residues 180 to 192, 230 to 290, 354 to 409, and 520 to 554 of SEQ ID NO: 13 that are consistent with 52999 belonging to the Peptidase_M8 family of zinc metallopeptidases (PFAM Accession PF01457; FIG. 11). The majority of zinc-dependent metallopeptidases (with the notable exception of the carboxypeptidases) such as the Peptidase_M8 family share a common pattern of primary structure in the part of their sequence involved in the binding of zinc, and can be grouped together as a superfamily, known as the metzincins, on the basis of this sequence similarity. From the tertiary structure of thermolysin, the position of the residues acting as zinc ligands and those involved in the catalytic activity are known. Two of the zinc ligands are histidines which are very close together in the sequence; C-terminal to the first histidine is a glutamic acid residue which acts as a nucleophile and promotes the attack of a water molecule on the carbonyl carbon of the substrate. A signature pattern which includes the two histidine and the glutamic acid residues is sufficient to detect this superfamily of proteins (Rawlings and Barrett (1995) Methods Enzymol. 248:183-228).

The 52999 protein includes such a zinc metallopeptidase zinc-binding signature sequence (ATLHELLHAL) from amino acids 272-281 of SEQ ID NO: 13 (ProSite PS0014/PDOC00129), consistent with the catalytic HEXXH zinc-binding motif of the zinc metallopeptidases.

For general information regarding PFAM identifiers, PS prefix and PF prefix domain identification numbers, refer to Sonnhammer et al. (1997) Protein 28:405-420 and www.psc.edu/general/software/packages/pfam/pfam.html.

The 52999 protein also contains predicted transmembrane domains that extend from about amino acid 632-649 and 706-722 of SEQ ID NO: 13.

As used herein, the term “metallopeptidase” refers to a protein or polypeptide that is capable of catalyzing the cleavage of a polypeptide bond through hydrolysis (i.e., possessing polypeptide hydrolytic activity) and contains at least one co-factor selected from the group consisting of Zn²⁺, Mn²⁺, Mg²⁺, and Ca²⁺. Metallopeptidases can have a specificity for various polypeptide substrates including a preference for hydrophobic residues at P1 and P1′ and basic residues at P2 and P3′. Based on the sequence similarities described above, the 52999 molecules of the present invention are predicted to have similar biological activities as metallopeptidase family members.

A plasmid containing the nucleotide sequence encoding human 52999 was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. § 112.

The 52999 protein contains a significant number of structural characteristics in common with members of the metallopeptidase family as described above.

As the biological functions of metallopeptidases include protein maturation and protein degradation, they typically play a role in diverse cellular processes. In particular, metallopeptidases have been shown to have a role in tumor growth, metastasis, and angiogenesis; in inflammatory disorders including, but not limited to osteoarthritis and rheumatoid arthritis, multiple sclerosis, Crohn disease, psoriasis, periodontal disease, and asthma; in macular degeneration; in restenosis; and in Alzheimer's disease.

A 52999 polypeptide can include a “metallopeptidase zinc-binding motif” or regions homologous with the “Peptidase_M8 family of metallopeptidases”.

As used herein, the term “Peptidase_M8 family of metallopeptidases” includes an amino acid sequence having a bit score for the alignment of the sequence to the Peptidase_M8 family domain (HMM) of at least 8. Preferably, a Peptidase_M8 family domain has a bit score for the alignment of the sequence to the metallopeptidase domain (HMM) of at least 16 or greater. The Peptidase_M8 family (HMM) has been assigned the PFAM Accession PF01457 (pfam.wustl.edu/). An alignment of the Peptidase_M8 family domain of human 52999 (amino acids 180 to 192, 230 to 290, 354 to 409, and 520 to 554 of SEQ ID NO: 13) with the consensus amino acid sequences derived from a hidden Markov model is depicted in FIG. 11.

In a preferred embodiment 52999 polypeptide or protein has regions with at least about 60%, 70%, 80%, 90%, 95%, 99%, or 100% homology with the Peptidase_M8 family of metallopeptidases (e.g., amino acid residues 180 to 192, 230 to 290, 354 to 409, and 520 to 554 of SEQ ID NO: 13).

To identify the presence of a Peptidase M8 metallopeptidase region of homology in a 52999 protein sequence, and make the determination that a polypeptide or protein of interest has a particular profile, the amino acid sequence of the protein can be searched against a database of HMMs (e.g., the Pfam database, release 2.1) using the default parameters (www.sanger.ac.uk/Software/Pfam/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J Mol. Biol. 235:1501-1531; and Stultz et al. (1993) Protein Sci. 2:305-314, the contents of which are incorporated herein by reference.

In one embodiment, a 52999 protein includes at least one transmembrane domain. As used herein, the term “transmembrane domain” includes an amino acid sequence of at least about 15 amino acid residues in length that spans a phospholipid membrane. Transmembrane domains are rich in hydrophobic residues, and typically have an α-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example,pfam.wustl.edu/cgi-bin/getdesc?name=7tm-1, and Zagotta W. N. et al. (1996) Annual Rev. Neuronsci. 19:235-63, the contents of which are incorporated herein by reference.

In a preferred embodiment, a 52999 polypeptide or protein has at least one transmembrane domain or a region which includes at least 15, 16, 17, 18, 20, 22, 24, 25, 30, 35 or 40 amino acid residues and has at least about 60%, 70% 80% 90% 95%, 99%, or 100% homology with a “transmembrane domain,” e.g., at least one transmembrane domain of human 52999 (e.g., amino acid residues 632-649 and 706-722 of SEQ ID NO: 13).

In another embodiment, a 52999 protein includes at least one non-transmembrane domain. As used herein, “non-transmembrane domains” are domains that reside outside of the membrane. When referring to plasma membranes, non-transmembrane domains include extracellular domains (i.e., outside of the cell) and intracellular domains (i.e., within the cell). When referring to membrane-bound proteins found in intracellular organelles (e.g., mitochondria, endoplasmic reticulum, peroxisomes and microsomes), non-transmembrane domains include those domains of the protein that reside in the cytosol (i.e., the cytoplasm), the lumen of the organelle, or the matrix or the intermembrane space (the latter two relate specifically to mitochondria organelles). The C-terminal amino acid residue of a non-transmembrane domain is adjacent to an N-terminal amino acid residue of a transmembrane domain in a naturally-occurring 52999, or 52999-like protein.

In a preferred embodiment, a 52999 polypeptide or protein has two non-transmembrane domains wherein the larger of the non-transmembrane domains includes an amino acid sequence having at least about 100-300, 300-500, or 500-600 or more amino acid residues in length, and has at least about 60%, 70% 80% 90% 95%, 99% or 100% homology with the larger of the two non-transmembrane domains of human 52999 (e.g., residues 21-612 of SEQ ID NO: 13). Preferably, the non-transmembrane domain is capable of polypeptide hydrolytic activity.

A non-transmembrane domain located at the N-terminus of a 52999 protein or polypeptide is referred to herein as an “N-terminal non-transmembrane domain.” As used herein, an “N-terminal non-transmembrane domain” includes an amino acid sequence having at least about 1-300, 300-500, or 500-600 or more amino acid residues in length, and is located outside the boundaries of a membrane. For example, an N-terminal non-transmembrane domain is located at about amino acid residues 21-612 of SEQ ID NO: 13.

Similarly, a non-transmembrane domain located at the C-terminus of a 52999 protein or polypeptide is referred to herein as a “C-terminal non-transmembrane domain.” As used herein, an “C-terminal non-transmembrane domain” includes an amino acid sequence having at least about 1-15, 15-25, or 25-36 or more amino acid residues in length and is located outside the boundaries of a membrane. For example, a C-terminal non-transmembrane domain is located at about amino acid residues 723-758 of SEQ ID NO: 13.

A 52999 molecule can further include a signal sequence. As used herein, a “signal sequence” refers to a peptide of about 20-80 amino acid residues in length which occurs at the N-terminus of secretory and integral membrane proteins and which contains a majority of hydrophobic amino acid residues. For example, a signal sequence contains at least about 20-25, 25-50, or 50-80 amino acid residues and has at least about 40-90%, hydrophobic amino acid residues (e.g., alanine, valine, leucine, isoleucine, phenylalanine, tyrosine, tryptophan, or proline). Such a signal sequence, also referred to in the art as a “signal peptide”, serves to direct a protein containing such a sequence to a lipid bilayer. For example, in one embodiment, a 52999 protein contains a signal sequence of about amino acids 1-20 of SEQ ID NO: 13. The signal sequence is cleaved during processing of the metallopeptidase. The processed 52999 protein corresponds to amino acids 21 to 758 of SEQ ID NO: 13.

As the 52999 polypeptides of the invention may modulate 52999-mediated activities, they may be useful for developing novel diagnostic and therapeutic agents for 52999-mediated or related disorders, as described below.

As used herein, a “52999 activity”, “biological activity of 52999” or “functional activity of 52999”, refers to an activity exerted by a 52999 protein, polypeptide or nucleic acid molecule on e.g., a 52999-responsive cell or on a 52999 polypeptide substrate, as determined in vivo or in vitro. In one embodiment, a 52999 activity is a direct activity, such as an association with a 52999 target molecule. A “target molecule” or “binding partner” or “ligand” or “substrate” is a molecule with which a 52999 protein binds or interacts in nature, e.g., a polypeptide that a 52999 protein cleaves. A 52999 activity can also be an indirect activity, e.g., a cellular signaling activity mediated by interaction of the 52999 protein with a 52999 ligand. For example, the 52999 proteins of the present invention can have one or more of the following activities: 1) cleavage of a protein precursor to maturation; 2) cleavage of a proenzyme to its active state; 3) catalysis of protein degradation; 4) catalysis of the degradation of extracellular matrix proteins; 5) modulation of tumor cell growth and invasion; and 6) modulation of angiogenesis.

Accordingly, 52999 protein may be mediate various disorders, including cellular proliferative and/or differentiative disorders; inflammatory disorders including, but not limited to osteoarthritis and rheumatoid arthritis, multiple sclerosis, Crohn disease, psoriasis, periodontal disease, and asthma; macular degeneration; restenosis; and Alzheimer's disease (see above for examples of such disorders).

Human 21999

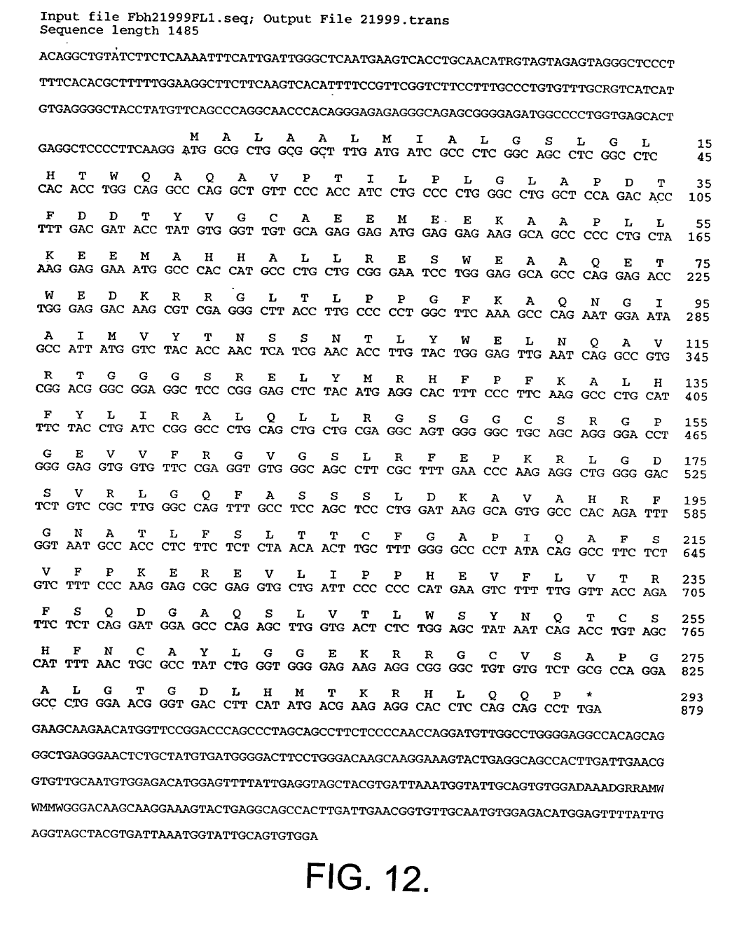

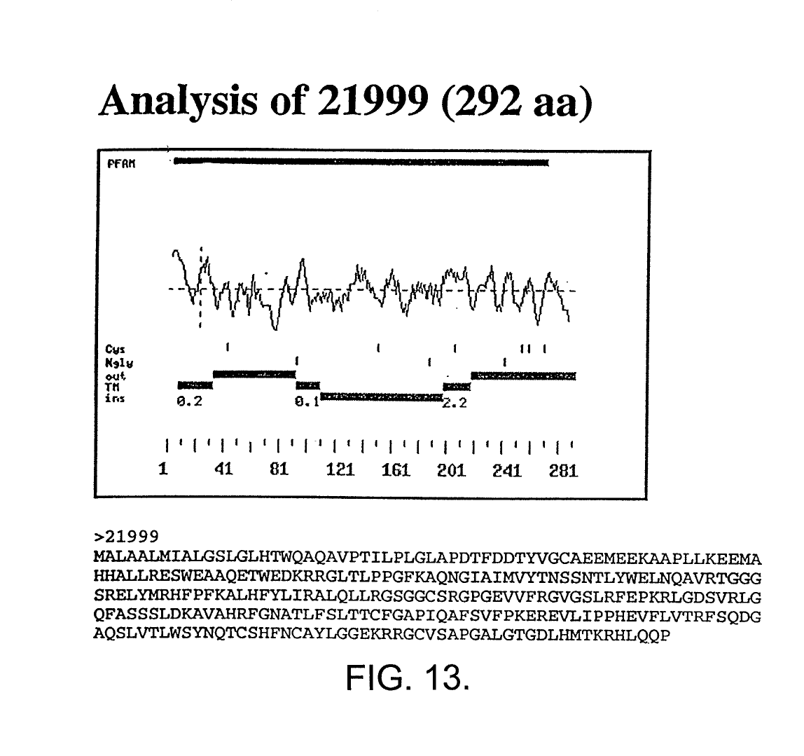

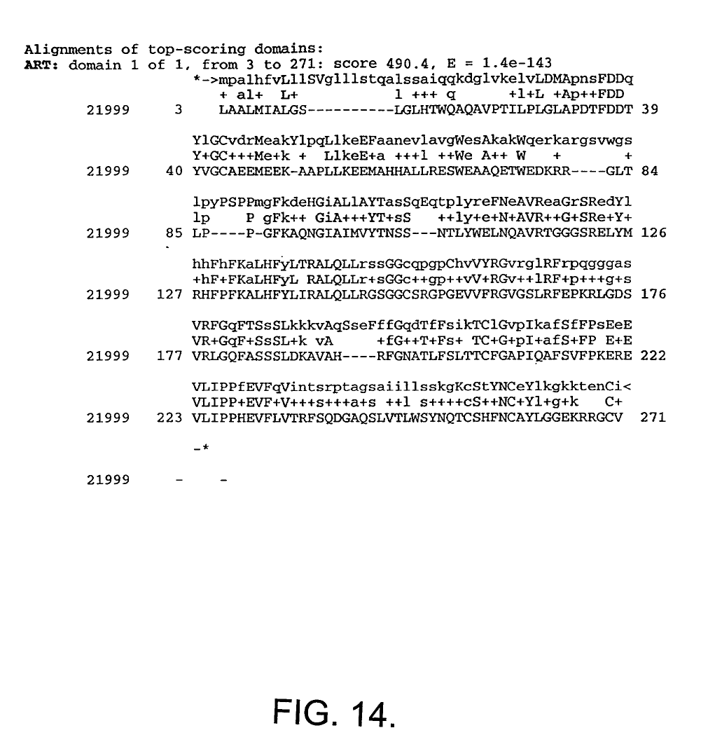

The human ADP-ribosyltransferase sequence (FIG. 12; SEQ ID NO: 19), which is approximately 1485 nucleotides long including untranslated regions, contains a predicted methionine-initiated coding sequence of about 879 nucleotides. The coding sequence encodes a 292 amino acid protein (SEQ ID NO: 20).

A plasmid containing the nucleotide sequence encoding human ADP-ribosyltransferase was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. § 112.

As the ADP-ribosyltransferase polypeptides of the invention may modulate ADP-ribosyltransferase-mediated activities, they may be useful as of for developing novel diagnostic and therapeutic agents for ADP-ribosyltransferase-mediated or related disorders, as described below.

As used herein, a “ADP-ribosyltransferase activity”, “biological activity of ADP-ribosyltransferase” or “functional activity of ADP-ribosyltransferase”, refers to an activity exerted by a ADP-ribosyltransferase protein, polypeptide or nucleic acid molecule on e.g., a ADP-ribosyltransferase-responsive cell or on a ADP-ribosyltransferase substrate, e.g., an ADP-ribose moiety substrate, as determined in vivo or in vitro. In one embodiment, a ADP-ribosyltransferase activity is a direct activity, such as an association with a ADP-ribosyltransferase target molecule. A “target molecule” or “binding partner” is a molecule with which a ADP-ribosyltransferase protein binds or interacts in nature, e.g., an ADP-ribose moiety of NAD.

ADP-ribosyltransferase protein can be detected in a variety of cell types, including viruses, bacteria and eukaryotic cells. ADP-ribosylation of target proteins by bacterial toxin transferases such as cholera, diptheria and pertussis toxins alters critical pathways. For example, cholera toxin ADP-ribosylates an arginine in the ∀-subunit of the stimulatory heterotrimeric guanine nucleotide-binding (G) protein, resulting in the activation of adenylyl cyclase and in increase in intracellular CHAMP. Eukaryotic ADP-ribosyltransferase activity has been detected in several tissues including human skeletal muscle. In fact, inhibitor studies suggest that the muscle transferase may participate in the regulation of myogenesis (Kharadia, S. V. et al. (1992) Exp. Cell. Res. 201: 33-42). Accordingly, the ADP-ribosyltransferase may be involved in various cellular metabolic and proliferative/differentiative disorders. Examples of cellular proliferative and/or differentiative disorders include cancer, e.g., carcinoma, sarcoma, metastatic disorders or hematopoietic neoplastic disorder.

The ADP-ribosyltransferase nucleic acid and protein of the invention can be used to treat and/or diagnose a variety of proliferative disorders (see above, for examples).

Disorders involving blood vessels include, but are not limited to, responses of vascular cell walls to injury, such as endothelial dysfunction and endothelial activation and intimal thickening; vascular diseases including, but not limited to, congenital anomalies, such as arteriovenous fistula, atherosclerosis, and hypertensive vascular disease, such as hypertension; inflammatory disease—the vasculitides, such as giant cell (temporal) arteritis, Takayasu arteritis, polyarteritis nodosa (classic), Kawasaki syndrome (mucocutaneous lymph node syndrome), microscopic polyanglitis (microscopic polyarteritis, hypersensitivity or leukocytoclastic anglitis), Wegener granulomatosis, thromboanglitis obliterans (Buerger disease), vasculitis associated with other disorders, and infectious arteritis; Raynaud disease; aneurysms and dissection, such as abdominal aortic aneurysms, syphilitic (luetic) aneurysms, and aortic dissection (dissecting hematoma); disorders of veins and lymphatics, such as varicose veins, thrombophlebitis and phlebothrombosis, obstruction of superior vena cava (superior vena cava syndrome), obstruction of inferior vena cava (inferior vena cava syndrome), and lymphangitis and lymphedema; tumors, including benign tumors and tumor-like conditions, such as hemangioma, lymphangioma, glomus tumor (glomangioma), vascular ectasias, and bacillary angiomatosis, and intermediate-grade (borderline low-grade malignant) tumors, such as Kaposi sarcoma and hemangloendothelioma, and malignant tumors, such as angiosarcoma and hemangiopericytoma; and pathology of therapeutic interventions in vascular disease, such as balloon angioplasty and related techniques and vascular replacement, such as coronary artery bypass graft surgery.

The 8035, 84242, 55304, 52999, and 21999 protein, fragments thereof, and derivatives and other variants of the sequence in SEQ ID NO: 2, SEQ ID NO: 6, SEQ ID NO: 10, SEQ ID NO: 13, and SEQ ID NO: 20, respectively, are collectively referred to as “polypeptides or proteins of the invention” or “8035, 84242, 55304, 52999, and 21999 polypeptides or proteins”. Nucleic acid molecules encoding such polypeptides or proteins are collectively referred to as “nucleic acids of the invention” or “8035, 84242, 55304, 52999, and 21999 nucleic acids.” 8035, 84242, 55304, 52999, and 21999 molecules refer to 8035 and 84242 nucleic acids, polypeptides, and antibodies.

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under stringent conditions” describes conditions for hybridization and washing. Stringent conditions are known to those skilled in the art and can be found in Current Protocols in Molecular Biology John Wiley & Sons, N.Y. (1989), 6.3.1-6.3.6. Aqueous and nonaqueous methods are described in that reference and either can be used. A preferred, example of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions are hybridization in 6× sodium chloride/sodium citrate (SSC) at about 45° C., followed by one or more washes in 0.2×SSC, 0.1% SDS at 60° C. Preferably, stringent hybridization conditions are hybridization in 6× sodium chloride/sodium citrate (SSC) at about 45° C., followed by one or more washes in 0.2×SSC, 0.1% SDS at 65° C. Particularly preferred stringency conditions (and the conditions that should be used if the practitioner is uncertain about what conditions should be applied to determine if a molecule is within a hybridization limitation of the invention) are 0.5M Sodium Phosphate, 7% SDS at 65° C., followed by one or more washes at 0.2×SSC, 1% SDS at 65° C. Preferably, an isolated nucleic acid molecule of the invention that hybridizes under stringent conditions to the sequence of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 14, SEQ ID NO: 19, or SEQ ID NO: 21 corresponds to a naturally-occurring nucleic acid molecule.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules that include an open reading frame encoding an 8035, 84242, 55304, 52999, or 21999 protein, preferably a mammalian 8035, 84242, 55304, 52999, or 21999 protein, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means a preparation of 8035, 84242, 55304, 52999, or 21999 protein having less than about 30%, 20%, 10% and more preferably 5% (by dry weight), of non-8035, 84242, 55304, 52999, or 21999 protein, respectively, (also referred to herein as a “contaminating protein”), or of chemical precursors or non-8035, 84242, 55304, 52999, or 21999 chemicals, respectively. When the 8035, 84242, 55304, 52999, and 21999 protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of 8035, 84242, 55304, 52999, or 21999 (e.g., the sequence of SEQ ID NO: 2, SEQ ID NO: 6, SEQ ID NO: 10, SEQ ID NO: 13, and SEQ ID NO: 20) without abolishing or more preferably, without substantially altering a biological activity, whereas an “essential” amino acid residue results in such a change. For example, amino acid residues that are conserved among the 84242 or 8035 polypeptides of the present invention, e.g., those present in the RING or IBR protein domains, are predicted to be particularly unamenable to alteration. Similarly, amino acid residues that are conserved among the 55304 polypeptides of the present invention, e.g., those present in the aminopeptidase domain, are predicted to be particularly unamenable to alteration. Amino acid residues that are conserved among the 52999 polypeptides of the present invention, in particular those present in the metal-binding active site domain, are also not predicted to be amenable to alteration. Amino acid residues that are conserved among the 21999 polypeptides of the present invention, e.g., those present in the transferase domain, are predicted to be particularly unamenable to alteration, as well.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 8035, 84242, 55304, 52999, or 21999 protein is preferably replaced with another amino acid residue from the same side chain family. Alternatively, in another embodiment, mutations can be introduced randomly along all or part of an 8035, 84242, 55304, 52999, or 21999 coding sequence, such as by saturation mutagenesis, and the resultant mutants can be screened for 8035, 84242, 55304, 52999, or 21999 biological activity to identify mutants that retain activity. Following mutagenesis of the sequence of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 14, SEQ ID NO: 19, or SEQ ID NO: 21, or the nucleotide sequences of the DNA inserts of the plasmids deposited with ATCC as Accession Number ______, ______, ______, ______, or _____, the encoded protein can be expressed recombinantly and the activity of the protein can be determined.

As used herein, a “biologically active portion” of an 8035, 84242, 55304, 52999, or 21999 protein includes a fragment of an 8035, 84242, 55304, 52999, or 21999 protein that participates in an interaction between an 8035, 84242, 55304, 52999, or 21999 molecule and a non-8035, non-84242, non-55304, non-52999, or non-21999 molecule. Biologically active portions of 8035, 84242, 55304, 52999, or 21999 proteins include peptides comprising amino acid sequences sufficiently homologous to or derived from the amino acid sequence of the 8035, 84242, 55304, 52999, or 21999 protein, e.g., the amino acid sequence shown in SEQ ID NO: 2, SEQ ID NO: 6, SEQ ID NO: 10, SEQ ID NO: 13, and SEQ ID NO: 20, respectively, that include less amino acids than the full length 8035, 84242, 55304, 52999, or 21999 protein, and exhibit at least one activity of an 8035, 84242, 55304, 52999, or 21999 protein. Typically, biologically active portions comprise a domain or motif with at least one activity of the 8035, 84242, 55304, 52999, or 21999 protein, e.g., in the case of 8035, or 84242, RING finger protein activity; in the case of 55304, aminopeptidase protein activity; in the case of 52999, metallopeptidase protein activity; and in the case of 21999, ribosyltransferase activity. A biologically active portion of an 8035, 84242, 55304, 52999, or 21999 protein can be a polypeptide that is, for example, 10, 25, 50, 100, 200 or more amino acids in length. Biologically active portions of an 8035, 84242, 55304, 52999, or 21999 protein can be used as targets for developing agents that modulate an 8035, 84242, 55304, 52999, or 21999 mediated activity, e.g., in the case of 8035 or 84242, RING finger protein activity; in the case of 55304, aminopeptidase protein activity; in the case of 52999, metallopeptidase protein activity; and in the case of 21999, ribosyltransferase activity.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence (e.g., when aligning a second sequence to the 8035 amino acid sequence of SEQ ID NO: 2 having 130 amino acid residues, at least 173, preferably at least 217, more preferably at least 260, even more preferably at least 303, and even more preferably at least 346, 390 or 433 amino acid residues are aligned; when aligning a second sequence to the 84242 amino acid sequence of SEQ ID NO: 6 having 121 amino acid residues, at least 161, preferably at least 202, more preferably at least 242, even more preferably at least 282, and even more preferably at least 322, 363 or 403 amino acid residues are aligned; when aligning a second sequence to the 55304 amino acid sequence of SEQ ID NO: 10 having 204 amino acid residues, at least 272, preferably at least, more preferably at least 340, even more preferably at least 408, and even more preferably at least 544, 612 or 680 amino acid residues are aligned; when aligning a second sequence to the 52999 amino acid sequence of SEQ ID NO: 13 having 227 amino acid residues, at least 303, preferably at least 379, more preferably at least 455, even more preferably at least 530, and even more preferably at least 606, 682 or 758 amino acid residues are aligned; and, when aligning a second sequence to the ADP-ribosyltransferase amino acid sequence of SEQ ID NO: 20 having 88 amino acid residues, at least 117, preferably at least 146, more preferably at least 177, even more preferably at least 204, and even more preferably at least 234, 263 or 292 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (1970) J. Mol. Biol. 48:444-453 algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) is using a Blossum 62 scoring matrix with a gap open penalty of 12, a gap extend penalty of 4, and a frameshift gap penalty of 5.

The percent identity between two amino acid or nucleotide sequences can be determined using the algorithm of E. Meyers and W. Miller (1989) CABIOS 4:11-17 which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to 8035, 84242, 55304, 52999, or 21999 nucleic acid molecules of the invention. BLAST protein searches can be performed with the XBLAST program, score=50, wordlength=3 to obtain amino acid sequences homologous to 8035, 84242, 55304, 52999, or 21999 protein molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

“Misexpression or aberrant expression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject”, as used herein, can refer to a mammal, e.g., a human, or to an experimental or animal or disease model. The subject can also be a non-human animal, e.g., a horse, cow, goat, or other domestic animal.

A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

Various aspects of the invention are described in further detail below.

Isolated Nucleic Acid Molecules

In one aspect, the invention provides, isolated or purified, nucleic acid molecules that encode an 8035 and 84242 polypeptide described herein, e.g., a full length 8035 and 84242 protein or a fragment thereof, e.g., a biologically active portion of 8035 and 84242 protein. Also included are nucleic acid fragments suitable for use as a hybridization probes, that can be used, e.g., to identify a nucleic acid molecule encoding a polypeptide of the invention, 8035 and 84242 MRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO: 1, or the nucleotide sequence of the DNA insert of the plasmid deposited with ATCC as Accession Number ______, or a portion of any of these nucleotide sequences. In one embodiment, an isolated nucleic acid molecule of the invention includes the nucleotide sequence shown in SEQ ID NO: 5, or the nucleotide sequence of the DNA insert of the plasmid deposited with ATCC as Accession Number ______, or a portion of any of these nucleotide sequences. In one embodiment, the nucleic acid molecule includes sequences encoding the human 8035 protein (i.e., “the coding region”, from nucleotides 613-1914 of SEQ ID NO: 1), as well as 5′ untranslated sequences (nucleotides 1-612 of SEQ ID NO: 1). In one embodiment, the nucleic acid molecule includes sequences encoding the human 84242 protein (i.e., “the coding region”, from nucleotides 744-1038 of SEQ ID NO: 5), as well as 5′ untranslated sequences (nucleotides 1-743 of SEQ ID NO: 5). Alternatively, the nucleic acid molecule can include only the coding region of SEQ ID NO: 1 or SEQ ID NO: 5 (e.g., nucleotides 613-1914 of SEQ ID NO: 1, corresponding to SEQ ID NO: 3, and nucleotides 744-1955 of SEQ ID NO: 5, corresponding to SEQ ID NO: 7, respectively) and, e.g., no flanking sequences which normally accompany the subject sequence. In another embodiment, the nucleic acid molecule encodes a sequence corresponding to the mature protein of SEQ ID NO: 2 or SEQ ID NO: 6.

In another embodiment, an isolated nucleic acid molecule of the invention includes a nucleic acid molecule which is a complement of the nucleotide sequence shown in SEQ ID NO: 1, SEQ ID NO: 3, SEQ ID NO: 5, SEQ ID NO: 7, or the nucleotide sequences of the DNA inserts of the plasmids deposited with ATCC as Accession Number ______ or ______, or a portion of any of these nucleotide sequences. In other embodiments, the nucleic acid molecule of the invention is sufficiently complementary to the nucleotide sequence shown in SEQ ID NO: 1, SEQ ID NO: 3, SEQ ID NO: 5, SEQ ID NO: 7, or the nucleotide sequences of the DNA inserts of the plasmids deposited with ATCC as Accession Number ______ or ______ such that it can hybridize to the nucleotide sequences shown in SEQ ID NO: 1, SEQ ID NO: 3, SEQ ID NO: 5, SEQ ID NO: 7, or the nucleotide sequences of the DNA inserts of the plasmids deposited with ATCC as Accession Number ______ or ______, thereby forming a stable duplex.

In one embodiment, an isolated nucleic acid molecule of the present invention includes a nucleotide sequence which is at least about 60%, 65%, 70%, 75%, 80%, 85%, 90%, 91%, 92%, 93%, 94%, 95%, 96%, 97%, 98%, 99%, or more homologous to the nucleotide sequence shown in SEQ ID NO: 1, SEQ ID NO: 3, SEQ ID NO: 5, SEQ ID NO: 7, or the nucleotide sequence of the DNA insert of the plasmid deposited with ATCC as Accession Number ______ or ______. In the case of an isolated nucleic acid molecule which is longer than or equivalent in length to the reference sequence, e.g., SEQ ID NO: 1 or SEQ ID NO: 3, and SEQ ID NO: 5 or SEQ ID NO: 7, the comparison is made with the full length of the reference sequence. Where the isolated nucleic acid molecule is shorter than the reference sequence, e.g., shorter than SEQ ID NO: 1 or SEQ ID NO: 3, and SEQ ID NO: 5 or SEQ ID NO: 7, the comparison is made to a segment of the reference sequence of the same length (excluding any loop required by the homology calculation).

In another aspect, the invention provides, an isolated or purified, nucleic acid molecule that encodes a 55304 polypeptide described herein, e.g., a full length 55304 protein or a fragment thereof, e.g., a biologically active portion of 55304 protein. Also included is a nucleic acid fragment suitable for use as a hybridization probe, which can be used, e.g., to a identify nucleic acid molecule encoding a polypeptide of the invention, 55304 mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO: 9, or the nucleotide sequence of the DNA insert of the plasmid deposited with ATCC as Accession Number ______, or a portion of any of these nucleotide sequences. In one embodiment, the nucleic acid molecule includes sequences encoding the human 55304 protein (i.e., “the coding region”, from nucleotides 803-2845 of SEQ ID NO: 9), as well as 5′ untranslated sequences (nucleotides 1-802 of SEQ ID NO: 9). Alternatively, the nucleic acid molecule can include only the coding region of SEQ ID NO: 9 (e.g., nucleotides 803-2845 of SEQ ID NO: 9, corresponding to SEQ ID NO: 11) and, e.g., no flanking sequences which normally accompany the subject sequence. In another embodiment, the nucleic acid molecule encodes a sequence corresponding to the mature protein of SEQ ID NO: 10.

In another embodiment, an isolated nucleic acid molecule of the invention includes a nucleic acid molecule which is a complement of the nucleotide sequence shown in SEQ ID NO: 9, SEQ ID NO: 11, or the nucleotide sequence of the DNA insert of the plasmid deposited with ATCC as Accession Number ______, or a portion of any of these nucleotide sequences. In other embodiments, the nucleic acid molecule of the invention is sufficiently complementary to the nucleotide sequence shown in SEQ ID NO: 9, SEQ ID NO: 11, or the nucleotide sequence of the DNA insert of the plasmid deposited with ATCC as Accession Number ______ such that it can hybridize to the nucleotide sequence shown in SEQ ID NO: 9, SEQ ID NO: 11, or the nucleotide sequence of the DNA insert of the plasmid deposited with ATCC as Accession Number ______, thereby forming a stable duplex.

In one embodiment, an isolated nucleic acid molecule of the present invention includes a nucleotide sequence which is at least about 60%, 65%, 70%, 75%, 80%, 85%, 90%, 91%, 92%, 93%, 94%, 95%, 96%, 97%, 98%, 99%, or more homologous to the nucleotide sequence shown in SEQ ID NO: 9, SEQ ID NO: 11, or the nucleotide sequence of the DNA insert of the plasmid deposited with ATCC as Accession Number ______. In the case of an isolated nucleic acid molecule which is longer than or equivalent in length to the reference sequence, e.g., SEQ ID NO: 9, or SEQ ID NO: 11, the comparison is made with the full length of the reference sequence. Where the isolated nucleic acid molecule is shorter than the reference sequence, e.g., shorter than SEQ ID NO: 9, or SEQ ID NO: 11, the comparison is made to a segment of the reference sequence of the same length (excluding any gap required by the alignment calculation).

In yet another aspect, the invention provides an isolated or purified nucleic acid molecule that encodes a 52999 polypeptide described herein, e.g., a full length 52999 protein or a fragment thereof, e.g., a biologically active portion of 52999 protein. Also included is a nucleic acid fragment suitable for use as a hybridization probe, which can be used, e.g., to a identify nucleic acid molecule encoding a polypeptide of the invention, 52999 MRNA, and fragments suitable for use as primers, e.g., PCR primers for the amplification or mutation of nucleic acid molecules.

In one embodiment, an isolated nucleic acid molecule of the invention includes the nucleotide sequence shown in SEQ I) NO: 12, or the nucleotide sequence of the DNA insert of the plasmid deposited with ATCC as Accession Number ______, or a portion of any of these nucleotide sequences. In one embodiment, the nucleic acid molecule includes sequences encoding the human 52999 protein (i.e., “the coding region”, from nucleotides 194-2470 of SEQ ID NO: 12), as well as 5′ untranslated sequences (nucleotides 1-193 of SEQ ID NO: 12). Alternatively, the nucleic acid molecule can include only the coding region of SEQ ID NO: 12 (e.g., nucleotides 194-2470 of SEQ ID NO: 12, corresponding to SEQ ID NO: 14) and, e.g., no flanking sequences which normally accompany the subject sequence. In another embodiment, the nucleic acid molecule encodes a sequence corresponding to the mature protein of SEQ ID NO: 13.

In another embodiment, an isolated nucleic acid molecule of the invention includes a nucleic acid molecule which is a complement of the nucleotide sequence shown in SEQ ID NO: 12, SEQ ID NO: 14, or the nucleotide sequence of the DNA insert of the plasmid deposited with ATCC as Accession Number ______, or a portion of any of these nucleotide sequences. In other embodiments, the nucleic acid molecule of the invention is sufficiently complementary to the nucleotide sequence shown in SEQ ID NO: 12, SEQ ID NO: 14, or the nucleotide sequence of the DNA insert of the plasmid deposited with ATCC as Accession Number ______ such that it can hybridize to the nucleotide sequence shown in SEQ ID NO: 12, SEQ ID NO: 14, or the nucleotide sequence of the DNA insert of the plasmid deposited with ATCC as Accession Number ______, thereby forming a stable duplex.

In one embodiment, an isolated nucleic acid molecule of the present invention includes a nucleotide sequence which is at least about 60%, 65%, 70%, 75%, 80%, 85%, 90%, 91%, 92%, 93%, 94%, 95%, 96%, 97%, 98%, 99%, or more homologous to the nucleotide sequence shown in SEQ ID NO: 12, SEQ ID NO: 14, or the nucleotide sequence of the DNA insert of the plasmid deposited with ATCC as Accession Number ______. In the case of an isolated nucleic acid molecule which is longer than or equivalent in length to the reference sequence, e.g., SEQ ID NO: 12, or SEQ ID NO: 14, the comparison is made with the full length of the reference sequence. Where the isolated nucleic acid molecule is shorter than the reference sequence, e.g., shorter than SEQ ID NO: 12, or SEQ ID NO: 14, the comparison is made to a segment of the reference sequence of the same length (excluding any loop required by the homology calculation).

In an additional aspect, the invention provides, an isolated or purified, nucleic acid molecule that encodes a ADP-ribosyltransferase polypeptide described herein, e.g., a full length ADP-ribosyltransferase protein or a fragment thereof, e.g., a biologically active portion of ADP-ribosyltransferase protein. Also included is a nucleic acid fragment suitable for use as a hybridization probe, which can be used, e.g., to a identify nucleic acid molecule encoding a polypeptide of the invention, ADP-ribosyltransferase mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO: 19, or the nucleotide sequence of the DNA insert of the plasmid deposited with ATCC as Accession Number ______, or a portion of any of these nucleotide sequences. In one embodiment, the nucleic acid molecule includes sequences encoding the human ADP-ribosyltransferase protein (i.e., “the coding region”, from nucleotides 255 to 1133 of SEQ ID NO: 19), as well as 5′ untranslated sequences of SEQ ID NO: 19. Alternatively, the nucleic acid molecule can include only the coding region of SEQ ID NO: 19 and, e.g., no flanking sequences which normally accompany the subject sequence. In another embodiment, the nucleic acid molecule encodes a sequence corresponding to the mature protein of SEQ ID NO: 20.

In another embodiment, an isolated nucleic acid molecule of the invention includes a nucleic acid molecule which is a complement of the nucleotide sequence shown in SEQ ID NO: 19, or the nucleotide sequence of the DNA insert of the plasmid deposited with ATCC as Accession Number ______, or a portion of any of these nucleotide sequences. In other embodiments, the nucleic acid molecule of the invention is sufficiently complementary to the nucleotide sequence shown in SEQ ID NO: 19, SEQ ID NO: 21, or the nucleotide sequence of the DNA insert of the plasmid deposited with ATCC as Accession Number ______ such that it can hybridize to the nucleotide sequence shown in SEQ ID NO: 19 or the nucleotide sequence of the DNA insert of the plasmid deposited with ATCC as Accession Number ______, thereby forming a stable duplex.

In one embodiment, an isolated nucleic acid molecule of the present invention includes a nucleotide sequence which is at least about 60%, 65%, 70%, 75%, 80%, 85%, 90%, 91%, 92%, 93%, 94%, 95%, 96%, 97%, 98%, 99%, or more homologous to the nucleotide sequence shown in SEQ ID NO: 19 or the nucleotide sequence of the DNA insert of the plasmid deposited with ATCC as Accession Number ______. In the case of an isolated nucleic acid molecule which is longer than or equivalent in length to the reference sequence, e.g., SEQ ID NO: 19, or the comparison is made with the full length of the reference sequence. Where the isolated nucleic acid molecule is shorter than the reference sequence, e.g., shorter than SEQ ID NO: 19, or the comparison is made to a segment of the reference sequence of the same length (excluding any loop required by the homology calculation).

8035 and 84242 Nucleic Acid Fragments

A nucleic acid molecule of the invention can include only a portion of the nucleic acid sequence of SEQ ID NO: 1, SEQ ID NO: 3, SEQ ID NO: 5, SEQ ID NO: 7, or the nucleotide sequence of the DNA insert of the plasmid deposited with ATCC as Accession Number ______ or ______. For example, such a nucleic acid molecule can include a fragment which can be used as a probe or primer or a fragment encoding a portion of an 8035 or 84242 protein, e.g., an immunogenic or biologically active portion of an 8035 or 84242 protein. A fragment can comprise nucleotides 1750-1875 of SEQ ID NO: 1, that encodes a RING finger protein domain of human 8035. A fragment can comprise nucleotides 837-1038 of SEQ ID NO: 5, that encodes an IBR protein domain of human 84242. The nucleotide sequences determined from the cloning of the 8035 and 84242 genes allow for the generation of probes and primers designed for use in identifying and/or cloning other 8035 and 84242 family members, or fragments thereof, as well as 8035 and 84242 homologues, or fragments thereof, from other species.

In another embodiment, a nucleic acid includes a nucleotide sequence that includes part, or all, of the coding regions and extends into either (or both) the 5′ or 3′ noncoding regions. Other embodiments include a fragment that includes a nucleotide sequence encoding an amino acid fragment described herein. Nucleic acid fragments can encode a specific domain or site described herein or fragments thereof, or fragments comprising a specific domain or site described herein that are at least 150 amino acids in length. Fragments also include nucleic acid sequences corresponding to specific amino acid sequences described above or fragments thereof. Nucleic acid fragments should not be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, the nucleic acid fragment can include a RING finger protein domain and/or an IBR protein domain. In a preferred embodiment the fragment is at least, 50, 75, 100, 125, 150, 200, 250, 300, 400, 500, 600, 700, 900, 1100, 1200 or 1300 base pairs in length.

8035 and 84242 probes and primers are provided. Typically a probe/primer is an isolated or purified oligonucleotide. The oligonucleotide typically includes a region of nucleotide sequence that hybridizes under stringent conditions to at least about 7, 12 or 15, preferably about 20 or 25, more preferably about 30, 35, 40, 45, 50, 55, 60, 65, or 75 consecutive nucleotides of a sense or antisense sequence of SEQ ID NO: 1, SEQ ID NO: 3, of SEQ ID NO: 5, SEQ ID NO: 7 or the nucleotide sequence of the DNA insert of the plasmid deposited with ATCC as Accession Number ______ or ______, or of a naturally occurring allelic variant or mutant of SEQ ID NO: 1, SEQ ID NO: 3, of SEQ ID NO: 5, SEQ ID NO: 7 or the nucleotide sequence of the DNA insert of the plasmid deposited with ATCC as Accession Number ______ or______.

In a preferred embodiment the nucleic acid is a probe that is at least 5 or 10, and less than 200, more preferably less than 100, or less than 50, base pairs in length. It should be identical, or differ by 1, or less than in 5 or 10 bases, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid that encodes a RING finger protein domain and/or an IBR protein domain (e.g., about amino acid residues 380-421 of SEQ ID NO: 2 and about amino acid residues 2-67 or 102-133 of SEQ ID NO: 6).

In another embodiment a set of primers is provided, e.g., primers suitable for use in a PCR, which can be used to amplify a selected region of an 8035 or 84242 sequence, e.g., a region described herein. The primers should be at least 5, 10, or 50 base pairs in length and less than 100, or less than 200, base pairs in length. The primers should be identical, or differ by one base from a sequence disclosed herein or from a naturally occurring variant. E.g., primers suitable for amplifying all or a portion of any of the following regions are provided: a RING finger protein domain (e.g., about amino acid residues 380-421 of SEQ ID NO: 2; an IBR protein domain (e.g., about amino acid residues 2-67 of SEQ ID NO: 6).

A nucleic acid fragment can encode an epitope bearing region of a polypeptide described herein.

A nucleic acid fragment encoding a “biologically active portion of an 8035 or 84242 polypeptide” can be prepared by isolating a portion of the nucleotide sequence of SEQ ID NO: 1, SEQ ID NO: 3, SEQ ID NO: 5, SEQ ID NO: 7 or the nucleotide sequence of the DNA insert of the plasmid deposited with ATCC as Accession Number ______ or ______, that encodes a polypeptide having an 8035 or 84242 biological activity (e.g., the biological activities of the 8035 and 84242 proteins as described herein), expressing the encoded portion of the 8035 or 84242 protein (e.g., by recombinant expression in vitro) and assessing the activity of the encoded portion of the 8035 or 84242 protein. For example, a nucleic acid fragment encoding a biologically active portion of 8035 includes an RING finger protein domain (e.g., about amino acid residues 380-421 of SEQ I) NO: 2). A nucleic acid fragment encoding a biologically active portion of an 8035 polypeptide, may comprise a nucleotide sequence which is greater than 125-1200 or more nucleotides in length. For example, a nucleic acid fragment encoding a biologically active portion of 84242 includes an IBR domain and/or a RING finger protein domain (e.g., about amino acid residues 2-67 and 102-133 of SEQ ID NO: 6, respectively). A nucleic acid fragment encoding a biologically active portion of an 84242 polypeptide, may comprise a nucleotide sequence which is greater than 125-1200 or more nucleotides in length.

In preferred embodiments, nucleic acids include a nucleotide sequence which is about 125, 150, 200, 300, 500, 600, 700, 800, 900, 1000, 1100, 1200, 1300, or 1302 nucleotides in length in the case of 8035 and which is about 125, 150, 200, 300, 500, 600, 700, 800, 900, 1000, 1100, 1200, or 1212 nucleotides in length in the case of 84242 and hybridizes under stringent hybridization conditions to a nucleic acid molecule of SEQ ID NO: 1, SEQ ID NO: 3, SEQ ID NO: 5, SEQ ID NO: 7, or the nucleotide sequence of the DNA insert of the plasmid deposited with ATCC as Accession Number ______ or ______.

55304 Nucleic Acid Fragments

A nucleic acid molecule of the invention can include only a portion of the nucleic acid sequence of SEQ ID NO: 9, SEQ ID NO: 11, or the nucleotide sequence of the DNA insert of the plasmid deposited with ATCC as Accession Number ______. For example, such a nucleic acid molecule can include a fragment which can be used as a probe or primer or a fragment encoding a portion of a 55304 protein, e.g., an immunogenic or biologically active portion of a 55304 protein. A fragment can comprise: nucleotides 803-2845 of SEQ ID NO: 9, which encodes an aminopeptidase domain of human 55304. The nucleotide sequence determined from the cloning of the 55304 gene allows for the generation of probes and primers designed for use in identifying and/or cloning other 55304 family members, or fragments thereof, as well as 55304 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 150 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, the nucleic acid fragment can include an Aminopeptidase domain. In a preferred embodiment the fragment is at least, 50, 100, 200, 300, 400, 500, 600, 700, 800, 900, 1000, or 1500 base pairs in length.

55304 probes and primers are provided. Typically a probe/primer is an isolated or purified oligonucleotide. The oligonucleotide typically includes a region of nucleotide sequence that hybridizes under stringent conditions to at least about 7, 12 or 15, preferably about 20 or 25, more preferably about 30, 35, 40, 45, 50, 55, 60, 65, or 75 consecutive nucleotides of a sense or antisense sequence of SEQ ID NO: 9, SEQ ID NO: 11, or the nucleotide sequence of the DNA insert of the plasmid deposited with ATCC as Accession Number ______, or of a naturally occurring allelic variant or mutant of SEQ ID NO: 9, SEQ ID NO: 11, or the nucleotide sequence of the DNA insert of the plasmid deposited with ATCC as Accession Number ______.

In a preferred embodiment the nucleic acid is a probe which is at least 5 or 10, and less than 200, more preferably less than 100, or less than 50, base pairs in length. It should be identical, or differ by 1, or less than in 5 or 10 bases, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes an Aminopeptidase domain (e.g., about amino acid residues 318-508 of SEQ ID NO: 10).

In another embodiment a set of primers is provided, e.g., primers suitable for use in a PCR, which can be used to amplify a selected region of a 55304 sequence, e.g., a region described herein. The primers should be at least 5, 10, or 50 base pairs in length and less than 100, or less than 200, base pairs in length. The primers should be identical, or differs by one base from a sequence disclosed herein or from a naturally occurring variant. E.g., primers suitable for amplifying all or a portion of any of the following regions are provided: an Aminopeptidase domain (e.g., about amino acid residues 318-508 of SEQ ID NO: 10).

A nucleic acid fragment can encode an epitope bearing region of a polypeptide described herein.

A nucleic acid fragment encoding a “biologically active portion of a 55304 polypeptide” can be prepared by isolating a portion of the nucleotide sequence of SEQ ID NO: 9, SEQ ID NO: 11, or the nucleotide sequence of the DNA insert of the plasmid deposited with ATCC as Accession Number ______, which encodes a polypeptide having a 55304 biological activity (e.g., the biological activities of the 55304 proteins as described herein), expressing the encoded portion of the 55304 protein (e.g., by recombinant expression in vitro) and assessing the activity of the encoded portion of the 55304 protein. For example, a nucleic acid fragment encoding a biologically active portion of 55304 includes an aminopeptidase domain (e.g., about amino acid residues 318-508 of SEQ ID NO: 10). A nucleic acid fragment encoding a biologically active portion of a 55304 polypeptide, may comprise a nucleotide sequence which is greater than 300-1200 or more nucleotides in length.

In preferred embodiments, nucleic acids include a nucleotide sequence which is about 300, 400, 500, 600, 700, 800, 900, 1000, 1100, 1200, 1300, 1400, 1500, 1600, 1700, 1800, 1900, 2000, or 2043 nucleotides in length and hybridizes under stringent hybridization conditions to a nucleic acid molecule of SEQ ID NO: 9, or SEQ ID NO: 11, or the nucleotide sequence of the DNA insert of the plasmid deposited with ATCC as Accession Number ______.

52999 Nucleic Acid Fragments

A nucleic acid molecule of the invention can include only a portion of the nucleic acid sequence of SEQ ID NO: 12, SEQ ID NO: 14, or the nucleotide sequence of the DNA insert of the plasmid deposited with ATCC as Accession Number ______. For example, such a nucleic acid molecule can include a fragment which can be used as a probe or primer or a fragment encoding a portion of a 52999 protein, e.g., an immunogenic or biologically active portion of a 52999 protein. A fragment can comprise all or a portion of the nucleotides from about nucleotide 253-2086 of SEQ ID NO: 12, that encode a polypeptide hydrolytic domain of human 52999. The nucleotide sequence determined from the cloning of the 52999 gene allows for the generation of probes and primers designed for use in identifying and/or cloning other 52999 family members, or fragments thereof, as well as 52999 homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 150 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a region or functional site described herein. A nucleic acid fragment can also include one or more regions or functional sites described herein. Thus, for example, a nucleic acid fragment can include a polypeptide hydrolytic domain or a conserved region or motif. In a preferred embodiment the fragment is at least 50, 100, 200, 400, 600, 800, 1000, 1200, 1400, 1600, 1800 or more base pairs in length.

52999 probes and primers are provided. Typically a probe/primer is an isolated or purified oligonucleotide. The oligonucleotide typically includes a region of nucleotide sequence that hybridizes under stringent conditions to at least about 7, 12 or 15, preferably about 20 or 25, more preferably about 30, 35, 40, 45, 50, 55, 60, 65, or 75 consecutive nucleotides of a sense or antisense sequence of SEQ ID NO: 12, SEQ ID NO: 14, or the nucleotide sequence of the DNA insert of the plasmid deposited with ATCC as Accession Number ______, or of a naturally occurring allelic variant or mutant of SEQ ID NO: 12, SEQ ID NO: 14, or the nucleotide sequence of the DNA insert of the plasmid deposited with ATCC as Accession Number ______.

In a preferred embodiment the nucleic acid is a probe which is at least 5 or 10, and less than 200, more preferably less than 100, or less than 50, base pairs in length. It should be identical, or differ by 1, or less than in 5 or 10 bases, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes a portion of an endopepdidase domain (e.g., about amino acid residues 21-631 of SEQ ID NO: 13).

In another embodiment a set of primers is provided, e.g., primers suitable for use in a PCR, which can be used to amplify a selected region of a 52999 sequence, e.g., a region described herein. The primers should be at least 5, 10, or 50 base pairs in length and less than 100, or less than 200, base pairs in length. The primers should be identical, or differs by one base from a sequence disclosed herein or from a naturally occurring variant. E.g., primers suitable for amplifying all or a portion of any of a polypeptide hydrolytic domain (e.g., about amino acid residues 21-631 of SEQ ID NO: 13).

A nucleic acid fragment can encode an epitope bearing region of a polypeptide described herein.

A nucleic acid fragment encoding a “biologically active portion of a 52999 polypeptide” can be prepared by isolating a portion of the nucleotide sequence of SEQ ID NO: 12, SEQ ID NO: 14, or the nucleotide sequence of the DNA insert of the plasmid deposited with ATCC as Accession Number ______, which encodes a polypeptide having a 52999 biological activity (e.g., the biological activities of the 52999 proteins as described herein), expressing the encoded portion of the 52999 protein (e.g., by recombinant expression in vitro) and assessing the activity of the encoded portion of the 52999 protein. For example, a nucleic acid fragment encoding a biologically active portion of 52999 may include a polypeptide hydrolytic domain (e.g., about amino acid residues 21-631 of SEQ ID NO: 13). A nucleic acid fragment encoding a biologically active portion of a 52999 polypeptide, may comprise a nucleotide sequence that is 300 -1800 or more nucleotides in length.

In preferred embodiments, nucleic acids include a nucleotide sequence that is about 300, 400, 500, 600, 700, 800, 900, 1000, 1100, 1200, 1300, 1400, 1600, 1800, 2000, 2200 or 2277 nucleotides in length and hybridizes under stringent hybridization conditions to a nucleic acid molecule of SEQ ID NO: 12, or SEQ ID NO: 14, or the nucleotide sequence of the DNA insert of the plasmid deposited with ATCC as Accession Number ______.

21999 Nucleic Acid Fragments

A nucleic acid molecule of the invention can include only a portion of the nucleic acid sequence of SEQ ID NO: 19, or the nucleotide sequence of the DNA insert of the plasmid deposited with ATCC as Accession Number ______. For example, such a nucleic acid molecule can include a fragment which can be used as a probe or primer or a fragment encoding a portion of a ADP-ribosyltransferase protein, e.g., an immunogenic or biologically active portion of a ADP-ribosyltransferase protein. A fragment can comprise nucleotide sequences which code for a portion of the ADP-ribosyltransferase protein of SEQ ID NO: 20 and retains biological activity. The nucleotide sequence determined from the cloning of the ADP-ribosyltransferase gene allows for the generation of probes and primers designed for use in identifying and/or cloning other ADP-ribosyltransferase family members, or fragments thereof, as well as ADP-ribosyltransferase homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 150 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. In a preferred embodiment the fragment is at least, 50, 100, 200, 300, 400, 500, 600, 700, or 900 base pairs in length.

ADP-ribosyltransferase probes and primers are provided. Typically a probe/primer is an isolated or purified oligonucleotide. The oligonucleotide typically includes a region of nucleotide sequence that hybridizes under stringent conditions to at least about 7, 12 or 15, preferably about 20 or 25, more preferably about 30, 35, 40, 45, 50, 55, 60, 65, or 75 consecutive nucleotides of a sense or antisense sequence of SEQ ID NO: 19, or the nucleotide sequence of the DNA insert of the plasmid deposited with ATCC as Accession Number ______, or of a naturally occurring allelic variant or mutant of SEQ ID NO: 19, , or the nucleotide sequence of the DNA insert of the plasmid deposited with ATCC as Accession Number ______.

In a preferred embodiment the nucleic acid is a probe which is at least 5 or 10, and less than 200, more preferably less than 100, or less than 50, base pairs in length. It should be identical, or differ by 1, or less than in 5 or 10 bases, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes a fragment of SEQ ID NO: 20.

In another embodiment a set of primers is provided, e.g., primers suitable for use in a PCR, which can be used to amplify a selected region of a ADP-ribosyltransferase sequence, e.g., a region described herein. The primers should be at least 5, 10, or 50 base pairs in length and less than 100, or less than 200, base pairs in length. The primers should be identical, or differs by one base from a sequence disclosed herein or from a naturally occurring variant. E.g., primers suitable for amplifying all or a portion of any of the region of SEQ ID NO: 20 are provided.

A nucleic acid fragment can encode an epitope bearing region of a polypeptide described herein.

A nucleic acid fragment encoding a “biologically active portion of a ADP-ribosyltransferase polypeptide” can be prepared by isolating a portion of the nucleotide sequence of SEQ ID NO: 19, or the nucleotide sequence of the DNA insert of the plasmid deposited with ATCC as Accession Number ______, which encodes a polypeptide having a ADP-ribosyltransferase biological activity (e.g., the biological activities of the ADP-ribosyltransferase proteins as described herein), expressing the encoded portion of the ADP-ribosyltransferase protein (e.g., by recombinant expression in vitro) and assessing the activity of the encoded portion of the ADP-ribosyltransferase protein.

In preferred embodiments, nucleic acids include a nucleotide sequence which is about 300, 400, 500, 600, 700, 800, or 879 nucleotides in length and hybridizes under stringent hybridization conditions to a nucleic acid molecule of SEQ ID NO: 19, or the nucleotide sequence of the DNA insert of the plasmid deposited with ATCC as Accession Number ______.

8035 and 84242 Nucleic Acid Variants

The invention further encompasses nucleic acid molecules that differ from the nucleotide sequence shown in SEQ ID NO: 1, SEQ ID NO: 3, SEQ ID NO: 5, SEQ ID NO: 7, or the nucleotide sequence of the DNA insert of the plasmid deposited with ATCC as Accession Number ______ or ______. Such differences can be due to degeneracy of the genetic code (and result in a nucleic acid which encodes the same 8035 or 84242 proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues than that that is shown in SEQ ID NO: 2 or SEQ ID NO: 6. If alignment is needed for this comparison the sequences should be aligned for maximum homology. “Looped” out sequences from deletions or insertions, or mismatches, are considered differences.

Nucleic acids of the invention can be chosen for having codons, which are preferred, or non preferred, for a particular expression system. E.g., the nucleic acid can be one in which at least one codon, preferably at least 10%, or 20% of the codons have been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO: 1, SEQ ID NO: 3, SEQ ID NO: 5, SEQ ID NO: 7, or the nucleotide sequence of the DNA insert of the plasmid deposited with ATCC as Accession Number ______ or ______, e.g., as follows: by at least one but less than 10, 20, 30, or 40 nucleotides; at least one but less than 1%, 5%, 10% or 20% of the in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the amino acid sequence shown in SEQ ID NO: 2 or SEQ ID NO: 6, or fragments of these sequences. Such nucleic acid molecules can readily be obtained as being able to hybridize under stringent conditions, to the nucleotide sequence shown in SEQ ID NO: 3 or SEQ ID NO: 7, or fragments of these sequences. Nucleic acid molecules corresponding to orthologs, homologs, and allelic variants of the 8035 and 84242 cDNAs of the invention can further be isolated by mapping to the same chromosome or locus as the 8035 or 84242 gene. Preferred variants include those that are correlated with RING finger protein activity (E3 ubiquitin ligase activity), e.g. variants that comprise nucleotide sequences encoding polypeptides that share identity to the amino acid sequence shown in SEQ ID NO: 2 or SEQ ID NO: 6 or a fragment of these sequences retain RING finger protein activity (E3 ubiquitin ligase activity).

Allelic variants of 8035 and 84242, e.g., human 8035 and 84242, include both functional and non-functional proteins. Functional allelic variants are naturally occurring amino acid sequence variants of the 8035 and 84242 proteins within a population that maintain the ability to function as E3 ubiquitin ligases. Functional allelic variants will typically contain only conservative substitution of one or more amino acids of SEQ ID NO: 2 or SEQ ID NO: 6 or substitution, deletion, or insertion of non-critical residues in non-critical regions of these proteins. Non-functional allelic variants are naturally-occurring amino acid sequence variants of 8035 and 84242, e.g., human 8035 and 84242, proteins within a population that do not have the ability function as E3 ubiquitin ligases. Non-functional allelic variants will typically contain a non-conservative substitution, a deletion, or insertion, or premature truncation of the amino acid sequence of SEQ ID NO: 2 or SEQ ID NO: 6, or a substitution, insertion, or deletion in critical residues or critical regions of these proteins.

Moreover, nucleic acid molecules encoding other 8035 and 84242 family members and, thus, which have a nucleotide sequence which differs from the 8035 and 84242 sequences of SEQ ID NO: 1, SEQ ID NO: 3, SEQ ID NO: 5, SEQ ID NO: 7, or the nucleotide sequence of the DNA insert of the plasmid deposited with ATCC as Accession Number ______ or ______ are intended to be within the scope of the invention.

55304 Nucleic Acid Variants

The invention further encompasses nucleic acid molecules that differ from the nucleotide sequence shown in SEQ ID NO: 9, SEQ ID NO: 11 , or the nucleotide sequence of the DNA insert of the plasmid deposited with ATCC as Accession Number ______. Such differences can be due to degeneracy of the genetic code (and result in a nucleic acid which encodes the same 55304 proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that is shown in SEQ ID NO: 10. If alignment is needed for this comparison the sequences should be aligned for maximum homology. “Looped” out sequences from deletions or insertions, or mismatches, are considered differences.

Nucleic acids of the invention can be chosen for having codons, which are preferred, or non preferred, for a particular expression system. E.g., the nucleic acid can be one in which at least one codon, and preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO: 9, SEQ ID NO: 11, or the nucleotide sequence of the DNA insert of the plasmid deposited with ATCC as Accession Number ______, e.g., as follows: by at least one but less than 10, 20, 30, or 40 nucleotides; at least one but less than 1%, 5%, 10% or 20% of the in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the amino acid sequence shown in SEQ ID NO: 10 or a fragment of this sequence. Such nucleic acid molecules can readily be obtained as being able to hybridize under stringent conditions, to the nucleotide sequence shown in SEQ ID NO: 11 or a fragment of this sequence. Nucleic acid molecules corresponding to orthologs, homologs, and allelic variants of the 55304 cDNAs of the invention can further be isolated by mapping to the same chromosome or locus as the 55304 gene. Preferred variants include those that are correlated with aminopeptidase activity.

Allelic variants of 55304, e.g., human 55304, include both functional and non-functional proteins. Functional allelic variants are naturally occurring amino acid sequence variants of the 55304 protein within a population that maintain the ability to hydrolyse the amino terminal amino acid from a peptide substrate. Functional allelic variants will typically contain only conservative substitution of one or more amino acids of SEQ ID NO: 10, or substitution, deletion or insertion of non-critical residues in non-critical regions of the protein. Non-functional allelic variants are naturally-occurring amino acid sequence variants of the 55304, e.g., human 55304, protein within a population that do not have the ability to remove the amino terminal amino acid from a peptide substrate. Non-functional allelic variants will typically contain a non-conservative substitution, a deletion, or insertion, or premature truncation of the amino acid sequence of SEQ ID NO: 10, or a substitution, insertion, or deletion in critical residues or critical regions of the protein.

Moreover, nucleic acid molecules encoding other 55304 family members and, thus, which have a nucleotide sequence which differs from the 55304 sequences of SEQ ID NO: 9, SEQ ID NO: 11, or the nucleotide sequence of the DNA insert of the plasmid deposited with ATCC as Accession Number ______ are intended to be within the scope of the invention.

52999 Nucleic Acid Variants

The invention further encompasses nucleic acid molecules that differ from the nucleotide sequence shown in SEQ ID NO: 12, SEQ ID NO: 14, or the nucleotide sequence of the DNA insert of the plasmid deposited with ATCC as Accession Number ______. Such differences can be due to degeneracy of the genetic code (and result in a nucleic acid which encodes the same 52999 proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that is shown in SEQ ID NO: 13. If alignment is needed for this comparison the sequences should be aligned for maximum homology. “Looped” out sequences from deletions or insertions, or mismatches, are considered differences.

Nucleic acids of the invention can be chosen for having codons, which are preferred, or non preferred, for a particular expression system. E.g., the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO: 12, SEQ ID NO: 14, or the nucleotide sequence of the DNA insert of the plasmid deposited with ATCC as Accession Number ______, e.g., as follows: by at least one but less than 10, 20, 30, or 40 nucleotides; at least one but less than 1%, 5%, 10% or 20% of the in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the amino acid sequence shown in SEQ ID NO: 13 or a fragment of this sequence. Such nucleic acid molecules can readily be obtained as being able to hybridize under stringent conditions, to the nucleotide sequence shown in SEQ ID NO: 14 or a fragment of this sequence. Nucleic acid molecules corresponding to orthologs, homologs, and allelic variants of the 52999 cDNAs of the invention can further be isolated by mapping to the same chromosome or locus as the 52999 gene. Preferred variants include those that are correlated with metallopeptidase activity, e.g. variants that comprise nucleotide sequences encoding polypeptides that share identity to the amino acid sequence shown in SEQ ID NO: 13 or a fragment of this sequence retain metallopeptidase activity.

Allelic variants of 52999, e.g., human 52999, include both functional and non-functional proteins. Functional allelic variants are naturally occurring amino acid sequence variants of the 52999 protein within a population that maintain polypeptide hydrolytic activity. Functional allelic variants will typically contain only conservative substitution of one or more amino acids of SEQ ID NO: 13, or substitution, deletion or insertion of non-critical residues in non-critical regions of the protein. Non-functional allelic variants are naturally-occurring amino acid sequence variants of the 52999, e.g., human 52999, protein within a population that do not have the ability to catalyze the cleavage of polypeptide bonds. Non-functional allelic variants will typically contain a non-conservative substitution, a deletion, or insertion, or premature truncation of the amino acid sequence of SEQ ID NO: 13, or a substitution, insertion, or deletion in critical residues or critical regions of the protein.

Moreover, nucleic acid molecules encoding other 52999 family members and, thus, which have a nucleotide sequence which differs from the 52999 sequences of SEQ ID NO: 12, SEQ ID NO: 14, or the nucleotide sequence of the DNA insert of the plasmid deposited with ATCC as Accession Number ______ are intended to be within the scope of the invention.

21999 Nucleic Acid Variants

The invention further encompasses nucleic acid molecules that differ from the nucleotide sequence shown in SEQ ID NO: 19 or the nucleotide sequence of the DNA insert of the plasmid deposited with ATCC as Accession Number ______. Such differences can be due to degeneracy of the genetic code (and result in a nucleic acid which encodes the same ADP-ribosyltransferase proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that is shown in SEQ ID NO: 20. If alignment is needed for this comparison the sequences should be aligned for maximum homology. “Looped” out sequences from deletions or insertions, or mismatches, are considered differences.

Nucleic acids of the invention can be chosen for having codons, which are preferred, or non preferred, for a particular expression system. E.g., the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO: 19, or the nucleotide sequence of the DNA insert of the plasmid deposited with ATCC as Accession Number ______, e.g., as follows: by at least one but less than 10, 20, 30, or 40 nucleotides; at least one but less than 1%, 5%, 10% or 20% of the in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the amino acid sequence shown in SEQ ID NO: 20 or a fragment of this sequence. Such nucleic acid molecules can readily be obtained as being able to hybridize under stringent conditions, to the nucleotide sequence shown in SEQ ID NO: 19 or a fragment of this sequence. Nucleic acid molecules corresponding to orthologs, homologs, and allelic variants of the ADP-ribosyltransferase cDNAs of the invention can further be isolated by mapping to the same chromosome or locus as the ADP-ribosyltransferase gene.

Allelic variants of ADP-ribosyltransferase, e.g., human ADP-ribosyltransferase, include both functional and non-functional proteins. Functional allelic variants are naturally occurring amino acid sequence variants of the ADP-ribosyltransferase protein within a population that maintain the ability to transfer an ADP-ribose moiety to an acceptor protein. Functional allelic variants will typically contain only conservative substitution of one or more amino acids of SEQ ID NO: 20, or substitution, deletion or insertion of non-critical residues in non-critical regions of the protein. Non-functional allelic variants are naturally-occurring amino acid sequence variants of the ADP-ribosyltransferase, e.g., human ADP-ribosyltransferase, protein within a population that do not have the ability to transfer an ADP-ribose moiety to an acceptor protein. Non-functional allelic variants will typically contain a non-conservative substitution, a deletion, or insertion, or premature truncation of the amino acid sequence of SEQ ID NO: 20, or a substitution, insertion, or deletion in critical residues or critical regions of the protein.

Moreover, nucleic acid molecules encoding other ADP-ribosyltransferase family members and, thus, which have a nucleotide sequence which differs from the ADP-ribosyltransferase sequences of SEQ ID NO: 19, or the nucleotide sequence of the DNA insert of the plasmid deposited with ATCC as Accession Number ______ are intended to be within the scope of the invention.

Antisense Nucleic Acid Molecules, Ribozymes and Modified 8035, 84242, 55304, 52999, or 21999 Nucleic Acid Molecules

In another aspect, the invention features, an isolated nucleic acid molecule which is antisense to 8035, 84242, 55304, 52999, or 21999. An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire 8035, 84242, 55304, 52999, or 21999 coding strand, or to only a portion thereof (e.g., the coding region of human 8035, 84242, 55304, 52999, or 21999 corresponding to SEQ ID NO: 3, SEQ ID NO: 7, SEQ ID NO: 11, SEQ ID NO: 14, SEQ ID NO: 21, respectively). In another embodiment, the antisense nucleic acid molecule is antisense to a “noncoding region” of the coding strand of a nucleotide sequence encoding 8035, 84242, 55304, 52999, or 21999 (e.g., the 5′ and 3′ untranslated regions).

An antisense nucleic acid can be designed such that it is complementary to the entire coding region of 8035, 84242, 55304, 52999, or 21999 mRNA, but more preferably is an oligonucleotide which is antisense to only a portion of the coding or noncoding region of 8035, 84242, 55304, 52999, or 21999 mRNA. For example, the antisense oligonucleotide can be complementary to the region surrounding the translation start site of 8035 or 84242 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding an 8035, 84242, 55304, 52999, or 21999 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An a-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for an 8035, 84242, 55304, 52999, or 21999-encoding nucleic acid can include one or more sequences complementary to the nucleotide sequence of an 8035, 84242, 55304, 52999, or 21999 cDNA disclosed herein (i.e., the sequence of SEQ ID NO: 1, SEQ ID NO: 3, SEQ ID NO: 5, SEQ ID NO: 7, SEQ ID NO: 9, SEQ ID NO: 11, SEQ ID NO: 12, SEQ ID NO: 14, SEQ ID NO: 19, or SEQ ID NO: 21, or the nucleotide sequences of the DNA inserts of the plasmids deposited with ATCC as Accession Number ______, ______, ______, ______, or ______), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in an 8035, 84242, 55304, 52999, or 21999-encoding mRNA. See, e.g., Cech et al. U.S. Pat. No. 4,987,071; and Cech et al. U.S. Pat. No. 5,116,742. Alternatively, 8035, 84242, 55304, 52999, or 21999 mRNA can be used to select a catalytic RNA having a specific ribonuclease activity from a pool of RNA molecules. See, e.g., Bartel, D. and Szostak, J. W. (1993) Science 261:1411-1418.

8035, 84242, 55304, 52999, or 21999 gene expression can be inhibited by targeting nucleotide sequences complementary to the regulatory region of the 8035, 84242, 55304, 52999, or 21999 (e.g., the 8035, 84242, 55304, 52999, or 21999 promoter and/or enhancers) to form triple helical structures that prevent transcription of the 8035, 84242, 55304, 52999, or 21999 gene in target cells. See generally, Helene, C. (1991) Anticancer Drug Des. 6(6):569-84; Helene, C. et al. (1992) Ann. N.Y. Acad. Sci. 660: 27-36; and Maher, L. J. (1992) Bioassays 14(12):807-15. The potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or calorimetric.

An 8035, 84242, 55304, 52999, or 21999 nucleic acid molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93:14670-675.

PNAs of 8035, 84242, 55304, 52999, or 21999 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 8035, 84242, 55304, 52999, or 21999 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to an 8035, 84242, 55304, 52999, or 21999 nucleic acid of the invention, two complementary regions one having a fluorophore and one a quencher such that the molecular beacon is useful for quantitating the presence of the 8035, 84242, 55304, 52999, or 21999 nucleic acid of the invention in a sample. Molecular beacon nucleic acids are described, for example, in Lizardi et al. U.S. Pat. No. 5,854,033; Nazarenko et al. U.S. Pat. No. 5,866,336, and Livak et al. U.S. Pat. No. 5,876,930.

Isolated 8035 and 84242 Polypeptides

In another aspect, the invention features, an isolated 8035 or 84242 protein, or fragment, e.g., a biologically active portion, for use as immunogens or antigens to raise or test (or more generally to bind) anti-8035 or anti-84242 antibodies. 8035 and 84242 protein can be isolated from cells or tissue sources using standard protein purification techniques. 8035 and 84242 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and postranslational events. The polypeptide can be expressed in systems, e.g., cultured cells, which result in substantially the same postranslational modifications present when expressed the polypeptide is expressed in a native cell, or in systems which result in the alteration or omission of postranslational modifications, e.g., glycosylation or cleavage, present when expressed in a native cell.

In a preferred embodiment, an 8035 or 84242 polypeptide has one or more of the following characteristics:

(i) it functions as an E3 ubiquitin ligase;

(ii) it has a molecular weight, e.g., a deduced molecular weight, amino acid composition or other physical characteristic of the polypeptide of SEQ ID NO: 2 or SEQ ID NO: 6;

(iii) it has an overall sequence identity of at least 50%, preferably at least 60%, more preferably at least 70, 80, 90, 91%, 92%, 93%, 94%, 95%, 96%, 97%, 98%, or 99%, with a polypeptide of SEQ ID NO: 2 or SEQ ID NO: 6;

(iv) it has at least one RING finger protein domain and/or an IBR protein domain which preferably have an overall sequence identity of about 70%, 80%, 90% or 95% with amino acid residues 380-421 of SEQ ID NO: 2 (RING), amino acid residues 2-67 of SEQ ID NO: 6 (IBR), or amino acid residues 102-133 of SEQ ID NO: 6 (RING);

(v) it has at least 70%, preferably 80%, and most preferably 95% of the cysteines found in the amino acid sequence of the native proteins.

In a preferred embodiment the 8035 or 84242 protein, or fragments thereof, differs from the corresponding sequence in SEQ ID NO: 2 or SEQ ID NO: 6, respectively. In one embodiment it differs by at least 1 but by less than 15, 10 or 5 amino acid residues. In another it differs from the corresponding sequence in SEQ ID NO: 2 or SEQ ID NO: 6 by at least 1 residue but less than 20%, 15%, 10% or 5% of the residues in it differ from the corresponding sequence in SEQ ID NO: 2 or SEQ ID NO: 6. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment the differences are not in the RING finger protein domain. In another preferred embodiment one or more differences are in non-active site residues, e.g. outside of the RING finger protein domain.

Other embodiments include proteins that contain one or more changes in amino acid sequence, e.g., a change in an amino acid residue which is not essential for activity. Such 8035 and 84242 proteins differ in amino acid sequence from SEQ ID NO: 2 and SEQ ID NO: 6, yet retain biological activity.

In one embodiment, a biologically active portion of an 8035 or 84242 protein includes a RING finger protein domain. In another embodiment, a biologically active portion of an 8035 or 84242 protein includes an IBR protein domain and a RING finger protein domain. Moreover, other biologically active portions, in which other regions of the proteins are deleted, can be prepared by recombinant techniques and evaluated for one or more of the functional activities of a native 8035 or 84242 protein.

In a preferred embodiment, the 8035 or 84242 protein has an amino acid sequence shown in SEQ ID NO: 2 or SEQ ID NO: 6, respectively. In other embodiments, the 8035 or 84242 protein is substantially identical to SEQ ID NO: 2 or SEQ ID NO: 6, respectively. In yet another embodiment, the 8035 or 84242 protein is substantially identical to SEQ ID NO: 2 or SEQ ID NO: 6 and retains the functional activity of the protein of SEQ ID NO: 2 or SEQ ID NO: 6, respectively, as described in detail above. Accordingly, in another embodiment, the 8035 or 84242 protein is a protein which includes an amino acid sequence at least about 60%, 65%, 70%, 75%, 80%, 85%, 90%, 91%, 92%, 93%, 94%, 95%, 96%, 97%, 98%, 99%, or more identical to SEQ ID NO: 2 or SEQ ID NO: 6, respectively.

Isolated 55304 Polypeptides

In another aspect, the invention features, an isolated 55304 protein, or fragment, e.g., a biologically active portion, for use as immunogens or antigens to raise or test (or more generally to bind) anti-55304 antibodies. 55304 protein can be isolated from cells or tissue sources using standard protein purification techniques. 55304 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and postranslational events. The polypeptide can be expressed in systems, e.g., cultured cells, which result in substantially the same postranslational modifications present when expressed the polypeptide is expressed in a native cell, or in systems which result in the alteration or omission of postranslational modifications, e.g., glycosylation or cleavage, present when expressed in a native cell.

In a preferred embodiment, a 55304 polypeptide has one or more of the following characteristics:

(i) it catalyzes the removal of an amino terminal amino acid from a peptide substrate;

(ii) it has a molecular weight, e.g., a deduced molecular weight, amino acid composition or other physical characteristic of the polypeptide of SEQ ID NO: 10;

(iii) it has an overall sequence identity of at least 50%, preferably at least 60%, more preferably at least 70, 80, 90, 91%, 92%, 93%, 94%, 95%, 96%, 97%, 98%, or 99%, with a polypeptide of SEQ ID NO: 10 as determined using the preferred algorithm described elsewhere herein;

(iv) it has an aminopeptidase domain which preferably has an overall sequence identity of about 68%, 75%, 81%, 87.5% or 93% with amino acid residues 318, 336, 344, 346, 363, 374, 397, 399, 410, 411, 412, 425, 448, 450, 489, and 508 of SEQ ID NO: 10, i.e. 11, 12, 13, 14, or 15 of these amino acids are conserved between the 5304 protein and the corresponding residues of the amino acid sequence set forth in SEQ ID NO: 10;

(v) it has at least 70%, preferably 80%, and most preferably 95% of the cysteines found amino acid sequence of the native protein.

In a preferred embodiment the 55304 protein, or fragment thereof, differs from the corresponding sequence in SEQ ID NO: 10. In one embodiment it differs by at least one but by less than 15, 10 or 5 amino acid residues. In another it differs from the corresponding sequence in SEQ ID NO: 10 by at least one residue but less than 20%, 15%, 10% or 5% of the residues in it differ from the corresponding sequence in SEQ ID NO: 10. (If this comparison requires alignment the sequences should be aligned for maximum homology, e.g. by the GAP algorithm described elsewhere herein. Gapped sequences from deletions or insertions, or mismatches, are considered differences.) The differences are, preferably, differences or changes at a non-essential residue or a conservative substitution. In a preferred embodiment the differences are not in the aminopeptidase domain. In another preferred embodiment one or more differences are in non-active site residues, e.g. outside of the aminopeptidase domain.

Other embodiments include a protein that contain one or more changes in amino acid sequence, e.g., a change in an amino acid residue which is not essential for activity. Such 55304 proteins differ in amino acid sequence from SEQ ID NO: 10, yet retain biological activity.

In one embodiment, a biologically active portion of a 55304 protein includes an Aminopeptidase domain. Moreover, other biologically active portions, in which other regions of the protein are deleted, can be prepared by recombinant techniques and evaluated for one or more of the functional activities of a native 55304 protein.

In a preferred embodiment, the 55304 protein has an amino acid sequence shown in SEQ ID NO: 10. In other embodiments, the 55304 protein is substantially identical to SEQ ID NO: 10. In yet another embodiment, the 55304 protein is substantially identical to SEQ ID NO: 10 and retains the functional activity of the protein of SEQ ID NO: 10, as described in detail above. Accordingly, in another embodiment, the 55304 protein is a protein which includes an amino acid sequence at least about 60%, 65%, 70%, 75%, 80%, 85%, 90%, 91%, 92%, 93%, 94%, 95%, 96%, 97%, 98%, 99%, or more identical to SEQ ID NO: 10.

Isolated 52999 Polypeptides

In another aspect, the invention features, an isolated 52999 protein, or fragment, e.g., a biologically active portion, for use as immunogens or antigens to raise or test (or more generally to bind) anti-52999 antibodies. 52999 protein can be isolated from cells or tissue sources using standard protein purification techniques. 52999 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and postranslational events. The polypeptide can be expressed in systems, e.g., cultured cells, which result in substantially the same postranslational modifications present when expressed the polypeptide is expressed in a native cell, or in systems which result in the alteration or omission of postranslational modifications, e.g., glycosylation or cleavage, present when expressed in a native cell.

In a preferred embodiment, a 52999 polypeptide has one or more of the following characteristics:

(i) it is capable of catalyzing the cleavage of a polypeptide through hydrolysis;

(ii) it has a molecular weight, e.g., a deduced molecular weight, amino acid composition or other physical characteristic of the polypeptide of SEQ ID NO: 13;

(iii) it has an overall sequence identity of at least 50%, preferably at least 60%, more preferably at least 70, 80, 90, 91%, 92%, 93%, 94%, 95%, 96%, 97%, 98%, or 99%, with a polypeptide of SEQ ID NO: 13;

(iv) it has a zinc-binding signature sequence that preferably has an overall sequence identity of about 70%, 80%, 90%, or 95% or more with amino acid residues 272-281 of SEQ ID NO: 13;

(v) it has at least 70%, preferably 80%, and most preferably 95% of the cysteines found amino acid sequence of the native protein.

In a preferred embodiment the 52999 protein, or fragment thereof, differs from the corresponding sequence in SEQ ID NO: 13. In one embodiment it differs by at least one but by less than 15, 10 or 5 amino acid residues. In another it differs from the corresponding sequence in SEQ ID NO: 13 by at least one residue but less than 20%, 15%, 10% or 5% of the residues in it differ from the corresponding sequence in SEQ ID NO: 13. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment the differences are not in the metallopeptidase domain. In another preferred embodiment one or more differences are in non-active site residues, e.g. outside of the metallopeptidase domain.

Other embodiments include a protein that contain one or more changes in amino acid sequence, e.g., a change in an amino acid residue which is not essential for activity. Such 52999 proteins differ in amino acid sequence from SEQ ID NO: 13, yet retain biological activity.

In one embodiment, a biologically active portion of a 52999 protein includes a polypeptide hydrolytic domain. In another embodiment, a biologically active portion of a 52999 protein includes a portion of the polypeptide hydrolytic domain that includes the zinc-binding signature sequence. Moreover, other biologically active portions, in which other regions of the protein are deleted, can be prepared by recombinant techniques and evaluated for the functional activities of a native 52999 protein.

In a preferred embodiment, the 52999 protein has an amino acid sequence shown in SEQ ID NO: 13. In other embodiments, the 52999 protein is substantially identical to SEQ ID NO: 13. In yet another embodiment, the 52999 protein is substantially identical to SEQ ID NO: 13 and retains the functional activity of the protein of SEQ ID NO: 13, as described in detail above. Accordingly, in another embodiment, the 52999 protein is a protein which includes an amino acid sequence at least about 60%, 65%, 70%, 75%, 80%, 85%, 90%, 91%, 92%, 93%, 94%, 95%, 96%, 97%, 98%, 99%, or more identical to SEQ ID NO: 13.

Isolated 21999 Polypeptides

In another aspect, the invention features, an isolated ADP-ribosyltransferase protein, or fragment, e.g., a biologically active portion, for use as immunogens or antigens to raise or test (or more generally to bind) anti-ADP-ribosyltransferase antibodies. ADP-ribosyltransferase protein can be isolated from cells or tissue sources using standard protein purification techniques. ADP-ribosyltransferase protein or fragments thereof can be produced by recombinant DNA techniques or synthesized chemically.

Polypeptides of the invention include those which arise as a result of the existence of multiple genes, alternative transcription events, alternative RNA splicing events, and alternative translational and postranslational events. The polypeptide can be expressed in systems, e.g., cultured cells, which result in substantially the same postranslational modifications present when expressed the polypeptide is expressed in a native cell, or in systems which result in the alteration or omission of postranslational modifications, e.g., glycosylation or cleavage, present when expressed in a native cell.

In a preferred embodiment, a ADP-ribosyltransferase polypeptide has one or more of the following characteristics:

(i) can act to transfer an ADP-ribose moiety of NAD to an acceptor protein;

(ii) it has a molecular weight, e.g., a deduced molecular weight, amino acid composition or other physical characteristic of the polypeptide of SEQ ID NO: 20;

(iii) it has an overall sequence identity of at least 50%, preferably at least 60%, more preferably at least 70, 80, 90, 91%, 92%, 93%, 94%, 95%, 96%, 97%, 98%, or 99%, with a polypeptide of SEQ ID NO: 20.

In a preferred embodiment the ADP-ribosyltransferase protein, or fragment thereof, differs from the corresponding sequence in SEQ ID NO: 20. In one embodiment it differs by at least one but by less than 15, 10 or 5 amino acid residues. In another it differs from the corresponding sequence in SEQ ID NO: 20 by at least one residue but less than 20%, 15%, 10% or 5% of the residues in it differ from the corresponding sequence in SEQ ID NO: 20. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment the differences are not in the transferase domain. In another preferred embodiment one or more differences are in non-active site residues, e.g. outside of the transferase domain.

Other embodiments include a protein that contain one or more changes in amino acid sequence, e.g., a change in an amino acid residue which is not essential for activity. Such ADP-ribosyltransferase proteins differ in amino acid sequence from SEQ ID NO: 20, yet retain biological activity.

In one embodiment, a biologically active portion of a ADP-ribosyltransferase protein includes a transferase domain. Moreover, other biologically active portions, in which other regions of the protein are deleted, can be prepared by recombinant techniques and evaluated for one or more of the functional activities of a native ADP-ribosyltransferase protein.

In a preferred embodiment, the ADP-ribosyltransferase protein has an amino acid sequence shown in SEQ ID NO: 20. In other embodiments, the ADP-ribosyltransferase protein is substantially identical to SEQ ID NO: 20. In yet another embodiment, the ADP-ribosyltransferase protein is substantially identical to SEQ ID NO: 20 and retains the functional activity of the protein of SEQ ID NO: 20, as described in detail above. Accordingly, in another embodiment, the ADP-ribosyltransferase protein is a protein which includes an amino acid sequence at least about 60%, 65%, 70%, 75%, 80%, 85%, 90%, 91%, 92%, 93%, 94%, 95%, 96%, 97%, 98%, 99%, or more identical to SEQ ID NO: 20.

8035, 84242 55304, 52999 or 21999 Chimeric or Fusion Proteins

In another aspect, the invention provides 8035, 84242, 55304, 52999, or 21999 chimeric or fusion proteins. As used herein, an 8035, 84242, 55304, 52999, or 21999 “chimeric protein” or “fusion protein” includes an 8035, 84242, 55304, 52999, or 21999 polypeptide linked to a non-8035, non-84242, non-55304, non-52999, or non-21999 polypeptide. A “non-8035, non-84242, non-55304, non-52999, or non-21999 polypeptide” refers to a polypeptide having an amino acid sequence corresponding to a protein which is not substantially homologous to the 8035, 84242, 55304, 52999, or 21999 protein, respectively, e.g., a protein which is different from the 8035, 84242, 55304, 52999, or 21999 protein and which is derived from the same or a different organism. The 8035, 84242, 55304, 52999, or 21999 polypeptides of the fusion proteins can correspond to all or a portion e.g., a fragment described herein of an 8035, 84242, 55304, 52999, or 21999 amino acid sequence. In a preferred embodiment, an 8035, 84242, 55304, 52999, or 21999 fusion protein includes at least one biologically active portion of an 8035, 84242, 55304, 52999, or 21999 protein. The non-8035, non-84242, non-55304, non-52999, or non-21999 polypeptide can be fused to the N-terminus or C-terminus of the 8035, 84242, 55304, 52999, or 21999 polypeptide.

The fusion protein can include a moiety which has a high affinity for a ligand. For example, the fusion protein can be a GST-8035, GST-84242, GST-55304, GST-52999, or GST-21999 fusion protein in which the 8035, 84242, 55304, 52999, or 21999 sequences are fused to the C-terminus of the GST sequences. Such fusion proteins can facilitate the purification of recombinant 8035, 84242, 55304, 52999, or 21999. Alternatively, the fusion protein can be an 8035, 84242, 55304, 52999, or 21999 protein containing a heterologous signal sequence at its N-terminus. In certain host cells (e.g., mammalian host cells), expression and/or secretion of 8035, 84242, 55304, 52999, or 21999 can be increased through use of a heterologous signal sequence.

Fusion proteins can include all or a part of a serum protein, e.g., an IgG constant region, or human serum albumin.

The 8035, 84242, 55304, 52999, or 21999 fusion proteins of the invention can be incorporated into pharmaceutical compositions and administered to a subject in vivo. The 8035, 84242, 55304, 52999, or 21999 fusion proteins can be used to affect the bioavailability of an 8035, 84242, 55304, 52999, or 21999 substrate. 8035, 84242, 55304, 52999, or 21999 fusion proteins may be useful therapeutically for the treatment of disorders caused by, for example, (i) aberrant modification or mutation of a gene encoding an 8035, 84242, 55304, 52999, or 21999 protein; (ii) misregulation of the 8035, 84242, 55304, 52999, or 21999 gene; and (iii) aberrant post-translational modification of an 8035, 84242, 55304, 52999, or 21999 protein. “Treatment” is herein defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A “therapeutic agent” as defined herein, includes, but is not limited to, small molecules, peptides, antibodies, ribozymes and antisense oligonucleotides.

Moreover, the 8035, 84242, 55304, 52999, or 21999-fusion proteins of the invention can be used as immunogens to produce anti-8035, anti-84242, anti-55304, anti-52999, or anti-21999 antibodies, respectively, in a subject, to purify 8035, 84242, 55304, 52999, or 21999 ligands and in screening assays to identify molecules which inhibit the interaction of 8035, 84242, 55304, 52999, or 21999 with an 8035, 84242, 55304, 52999, or 21999 substrate.

Expression vectors are commercially available that already encode a fusion moiety (e.g., a GST polypeptide). An 8035, 84242, 55304, 52999, or 21999-encoding nucleic acid can be cloned into such an expression vector such that the fusion moiety is linked in-frame to the 8035, 84242, 55304, 52999, or 21999 protein.

Variants of 8035, 84242. 55304. 52999. or 21999 Proteins

In another aspect, the invention also features a variant of an 8035, 84242, 55304, 52999, or 21999 polypeptide, e.g., which functions as an agonist (mimetics) or as an antagonist. Variants of the 8035, 84242, 55304, 52999, or 21999 proteins can be generated by mutagenesis, e.g., discrete point mutation, the insertion or deletion of sequences or the truncation of an 8035, 84242, 55304, 52999, or 21999 protein. An agonist of the 8035, 84242, 55304, 52999, or 21999 proteins can retain substantially the same, or a subset, of the biological activities of the naturally occurring form of an 8035, 84242, 55304, 52999, or 21999 protein. An antagonist of an 8035, 84242, 55304, 52999, or 21999 protein can inhibit one or more of the activities of the naturally occurring form of the 8035, 84242, 55304, 52999, or 21999 protein by, for example, competitively modulating an 8035, 84242, 55304, 52999, or 21999-mediated activity of an 8035, 84242, 55304, 52999, or 21999 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the 8035, 84242, 55304, 52999, or 21999 protein.

Variants of an 8035, 84242, 55304, 52999, or 21999 protein can be identified by screening combinatorial libraries of mutants, e.g., truncation mutants, of an 8035, 84242, 55304, 52999, or 21999 protein for agonist or antagonist activity.

Libraries of fragments e.g., N terminal, C terminal, or internal fragments, of an 8035, 84242, 55304, 52999, or 21999 protein coding sequence can be used to generate a variegated population of fragments for screening and subsequent selection of variants of an 8035, 84242, 55304, 52999, or 21999 protein.

Variants in which a cysteine residues is added or deleted or in which a residue which is glycosylated is added or deleted are particularly preferred.

Methods for screening gene products of combinatorial libraries made by point mutations or truncation, and for screening cDNA libraries for gene products having a selected property. Recursive ensemble mutagenesis (REM), a new technique which enhances the frequency of functional mutants in the libraries, can be used in combination with the screening assays to identify 8035, 84242, 55304, 52999, or 21999 variants (Arkin and Yourvan (1992) Proc. Natl. Acad. Sci. USA 89:7811-7815; Delgrave et al. (1993) Protein Engineering 6(3):327-331).

Cell based assays can be exploited to analyze a variegated 8035, 84242, 55304, 52999, or 21999 library. For example, a library of expression vectors can be transfected into a cell line, e.g., a cell line, which ordinarily responds to 8035, 84242, 55304, 52999, or 21999 in a substrate-dependent manner. The transfected cells are then contacted with 8035, 84242, 55304, 52999, or 21999 and the effect of the expression of the mutant on signaling by the 8035, 84242, 55304, 52999, or 21999 substrate can be detected. For example, where 8035 or 84242 is tested, detection can be accomplished by measuring RING finger protein-mediated activity; where 55304 is tested, detection can be accomplished by measuring aminopeptidase activity; where 52999 is tested, detection can be accomplished by measuring polypeptide hydrolytic activity; and where 21999 is tested, detection can be accomplished by measuring transferase activity in the reaction wherein NAD(+) and L-arginine are converted by the ADP-ribosyltransferase enzyme to form the end-products nicotinamide and N2-(ADP-D-ribosyl)-L-arginine. Plasmid DNA can then be recovered from the cells which score for inhibition, or alternatively, potentiation of signaling by the 8035, 84242, 55304, 52999, or 21999 substrate, and the individual clones further characterized.

In another aspect, the invention features a method of making an 8035, 84242, 55304, 52999, or 21999 polypeptide, e.g., a peptide having a non-wild type activity, e.g., an antagonist, agonist, or super agonist of a naturally occurring 8035, 84242, 55304, 52999, or 21999 polypeptide, e.g., a naturally occurring 8035, 84242, 55304, 52999, or 21999 polypeptide. The method includes: altering the sequence of a 8035, 84242, 55304, 52999, or 21999 polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of an 8035, 84242, 55304, 52999, or 21999 polypeptide having a biological activity of a naturally occurring 8035, 84242, 55304, 52999, or 21999 polypeptide. The method includes: altering the sequence, e.g., by substitution or deletion of one or more residues, of an 8035, 84242, 55304, 52999, or 21999 polypeptide, e.g., altering the sequence of a non-conserved region, or a domain or residue described herein, and testing the altered polypeptide for the desired activity.

Anti-8035 Anti-84242. Anti-55304, Anti-52999, and Anti-21999 Antibodies

In another aspect, the invention provides an anti-8035, anti-84242, anti-55304, anti-52999, and anti-21999 antibody. The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin.

The antibody can be a polyclonal, monoclonal, recombinant, e.g., a chimeric or humanized, fully human, non-human, e.g., murine, or single chain antibody. In a preferred embodiment it has effector function and can fix complement. The antibody can be coupled to a toxin or imaging agent.

A full-length 8035, 84242, 55304, 52999, or 21999 protein or, antigenic peptide fragment of 8035, 84242, 55304, 52999, or 21999 can be used as an immunogen or can be used to identify anti-8035, anti-84242, anti-55304, anti-52999, or anti-21999 antibodies made with other immunogens, e.g., cells, membrane preparations, and the like. The antigenic peptide of 8035, 84242, 55304, 52999, or 21999 should include at least 8 amino acid residues of the amino acid sequence shown in SEQ ID NO: 2, SEQ ID NO: 6, SEQ ID NO: 10, SEQ ID NO: 13, and SEQ ID NO: 20 respectively, and encompasses an epitope of 8035, 84242, 55304, 52999, or 21999. Preferably, the antigenic peptide includes at least 10 amino acid residues, more preferably at least 15 amino acid residues, even more preferably at least 20 amino acid residues, and most preferably at least 30 amino acid residues.

Fragments of 8035 which include, e.g., residues 350-390 of SEQ ID NO: 2 can be used to make, e.g., used as immunogens, or used to characterize the specificity of an antibody or antibodies against what are believed to be hydrophilic regions of the 8035 protein. Similarly, a fragment of 8035 which includes, e.g., residues 200-230 of SEQ ID NO: 2 can be used to make an antibody against what is believed to be a hydrophobic region of the 8035 protein; a fragment of 8035 which includes residues 380-421 of SEQ ID NO: 2 can be used to make an antibody against the RING finger protein region of the 8035 protein. Antibodies reactive with, or specific for, any of these regions, or other regions or domains described herein are provided.

Fragments of 84242 which include, e.g., residues 190-220 of SEQ ID NO: 6 can be used to make, e.g., used as immunogens, or used to characterize the specificity of an antibody or antibodies against what are believed to be hydrophilic regions of the 84242 protein. Similarly, a fragment of 84242 which includes, e.g., residues 115-150 of SEQ ID NO: 6 can be used to make an antibody against what is believed to be a hydrophobic region of the 84242 protein; a fragment of 84242 which includes residues 2-67 of SEQ ID NO: 6 can be used to make an antibody against the IBR protein region of the 84242 protein. Antibodies reactive with, or specific for, any of these regions, or other regions or domains described herein are provided.

Fragments of 55304 which include, e.g., residues 650-670 of SEQ ID NO: 10 of SEQ ID NO: 5 can be used to make, e.g., used as immunogens, or used to characterize the specificity of an antibody or antibodies against what are believed to be hydrophilic regions of the 55304 protein. Similarly, a fragment of 55304 which includes, e.g., residues 240-260 of SEQ ID NO: 10 can be used to make an antibody against what is believed to be a hydrophobic region of the 55304 protein; a fragment of 55304 which includes residues 318-508 of SEQ ID NO: 10 can be used to make an antibody against the aminopeptidase region of the 55304 protein.

Fragments of 52999 that include residues from about amino acid 291-320 of SEQ ID NO: 13 can be used to make, e.g., used as immunogens, or characterize the specificity of an antibody or antibodies against what are believed to be hydrophilic regions of the 52999 protein. Similarly, a fragment of 52999 that includes residues from about amino acid 321-345 of SEQ ID NO: 13 can be used to make an antibody against what is believed to be a hydrophobic region of the 52999 protein; a fragment of 52999 that includes residues from about amino acid 270-290 of SEQ ID NO: 13 can be used to make an antibody against the active site region of the 52999 protein.

Fragments of 21999 can be used to make immunogens, or used to characterize the specificity of an antibody or antibodies against what are believed to be hydrophilic regions of the ADP-ribosyltransferase protein.

In a preferred embodiment the antibody fails to bind an Fc receptor, e.g. it is a type which does not support Fc receptor binding or has been modified, e.g., by deletion or other mutation, such that is does not have a functional Fc receptor binding region.

Preferred epitopes encompassed by the antigenic peptide are regions of 8035, 84242, 55304, 52999, or 21999 that are located on the surface of the proteins, e.g., hydrophilic regions, as well as regions with high antigenicity. For example, an Emini surface probability analysis of the human 8035, 84242, 55304, 52999, or 21999 protein sequence can be used to indicate the regions that have a particularly high probability of being localized to the surface of the 8035, 84242, 55304, 52999, or 21999 protein, respectively, and are thus likely to constitute surface residues useful for targeting antibody production.

In a preferred embodiment the antibody binds an epitope on any domain or region on 8035, 84242, 55304, 52999, or 21999 proteins described herein.

Chimeric, humanized, but most preferably, completely human antibodies are desirable for applications which include repeated administration, e.g., therapeutic treatment (and some diagnostic applications) of human patients.

The anti-8035, anti-84242, anti-55304, anti-52999, or anti-21999 antibody can be a single chain antibody. A single-chain antibody (scFV) may be engineered (see, for example, Colcher, D. et al. (Jun. 30, 1999,) Ann. NY Acad. Sci.880:263-80; and Reiter, Y. (1996 Feb) Clin. Cancer Res.2(2):245-52). The single chain antibody can be dimerized or multimerized to generate multivalent antibodies having specificities for different epitopes of the same target 8035, 84242, 55304, 52999, or 21999 protein.

An anti-8035, anti-84242, anti-55304, anti-52999, or anti-21999 antibody (e.g., monoclonal antibody) can be used to isolate 8035, 84242, 55304, 52999, or 21999 by standard techniques, such as affinity chromatography or immunoprecipitation. Moreover, an anti-8035, anti-84242, anti-55304, anti-52999, or anti-21999 antibody can be used to detect 8035, 84242, 55304, 52999, or 21999 protein (e.g., in a cellular lysate or cell supernatant) in order to evaluate the abundance and pattern of expression of the protein. Anti-8035, anti-84242, anti-55304, anti-52999, or anti-21999 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³⁵I, ³⁵S or ³H.

Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include an 8035, 84242, 55304, 52999, or 21999 nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., 8035, 84242, 55304, 52999, or 21999 proteins, mutant forms of 8035, 84242, 55304, 52999, or 21999 proteins, fusion proteins, and the like).

The recombinant expression vectors of the invention can be designed for expression of 8035, 84242, 55304, 52999, or 21999 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in 8035, 84242, 55304, 52999, or 21999 activity assays, (e.g., direct assays or competitive assays described in detail below), or to generate antibodies specific for 8035, 84242, 55304, 52999, or 21999 proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six (6) weeks).

To maximize recombinant protein expression in E. coli is to express the protein in host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

The 8035, 84242, 55304, 52999, or 21999 expression vector can be a yeast expression vector, a vector for expression in insect cells, e.g., a baculovirus expression vector or a vector suitable for expression in mammalian cells.

When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes see Weintraub, H. et al. (1986) Antisense RNA as a molecular tool for genetic analysis, Reviews—Trends in Genetics, Vol. 1(1).

Another aspect the invention provides a host cell which includes a nucleic acid molecule described herein, e.g., an 8035, 84242, 55304, 52999, or 21999 nucleic acid molecule within a recombinant expression vector or an 8035, 84242, 55304, 52999, or 21999 nucleic acid molecule containing sequences which allow it to homologously recombine into a specific site of the host cell's genome. The terms “host cell” and “recombinant host cell” are used interchangeably herein. Such terms refer not only to the particular subject cell but rather also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an 8035, 84242, 55304, 52999, or 21999 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride. co-precipitation, DEAE-dextran-mediated transfection, lipofection, or electroporation.

A host cell of the invention can be used to produce (i.e., express) an 8035, 84242, 55304, 52999, or 21999 protein. Accordingly, the invention further provides methods for producing a 8035, 84242, 55304, 52999, or 21999 protein using the host cells of the invention. In one embodiment, the method includes culturing the host cell of the invention (into which a recombinant expression vector encoding an 8035, 84242, 55304, 52999, or 21999 protein has been introduced) in a suitable medium such that an 8035, 84242, 55304, 52999, or 21999 protein is produced. In another embodiment, the method further includes isolating an 8035, 84242, 55304, 52999, or 21999 protein from the medium or the host cell.

In another aspect, the invention features, a cell or purified preparation of cells which include an 8035, 84242, 55304, 52999, or 21999 transgene, or which otherwise misexpress 8035, 84242, 55304, 52999, or 21999. The cell preparation can consist of human or non-human cells, e.g., rodent cells, e.g., mouse or rat cells, rabbit cells, or pig cells. In preferred embodiments, the cell or cells include an 8035, 84242, 55304, 52999, or 21999 transgene, e.g., a heterologous form of an 8035, 84242, 55304, 52999, or 21999, e.g., a gene derived from humans (in the case of a non-human cell). The 8035, 84242, 55304, 52999, or 21999 transgene can be misexpressed, e.g., overexpressed or underexpressed. In other preferred embodiments, the cell or cells include a gene which misexpress an endogenous 8035, 84242, 55304, 52999, or 21999, e.g., a gene the expression of which is disrupted, e.g., a knockout. Such cells can serve as a model for studying disorders which are related to mutated or misexpressed 8035, 84242, 55304, 52999, or 21999 alleles or for use in drug screening.

In another aspect, the invention features, a human cell, e.g., a hematopoietic stem cell, transformed with nucleic acid which encodes a subject 8035, 84242, 55304, 52999, or 21999 polypeptide.

Also provided are cells or a purified preparation thereof, e.g., human cells, in which an endogenous 8035, 84242, 55304, 52999, or 21999 is under the control of a regulatory sequence that does not normally control the expression of the endogenous 8035, 84242, 55304, 52999, or 21999 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 8035, 84242, 55304, 52999, or 21999 gene. For example, an endogenous 8035, 84242, 55304, 52999, or 21999 gene, e.g., a gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published on May 16, 1991.

Transgenic Animals

The invention provides non-human transgenic animals. Such animals are useful for studying the function and/or activity of an 8035, 84242, 55304, 52999, or 21999 protein and for identifying and/or evaluating modulators of 8035, 84242, 55304, 52999, or 21999 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous 8035, 84242, 55304, 52999, or 21999 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of an 8035, 84242, 55304, 52999, or 21999 protein to particular cells. A transgenic founder animal can be identified based upon the presence of an 8035, 84242, 55304, 52999, or 21999 transgene in its genome and/or expression of 8035, 84242, 55304, 52999, or 21999 mRNA in tissues or cells of the animals. A transgenic founder animal can then be used to breed additional animals carrying the transgene. Moreover, transgenic animals carrying a transgene encoding an 8035, 84242, 55304, 52999, or 21999 protein can further be bred to other transgenic animals carrying other transgenes. 8035, 84242, 55304, 52999, or 21999 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed herein.

Uses

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express an 8035, 84242, 55304, 52999, or 21999 protein (e.g., via a recombinant expression vector in a host cell in gene therapy applications), to detect an 8035, 84242, 55304, 52999, or 21999 mRNA (e.g., in a biological sample) or a genetic alteration in an 8035, 84242, 55304, 52999, or 21999 gene, and to modulate 8035, 84242, 55304, 52999, or 21999 activity, as described further below. The 8035, 84242, 55304, 52999, or 21999 proteins can be used to treat disorders characterized by insufficient or excessive production of an 8035, 84242, 55304, 52999, or 21999 substrate or production of 8035, 84242, 55304, 52999, or 21999 inhibitors. In addition, the 8035, 84242, 55304, 52999, or 21999 proteins can be used to screen for naturally occurring 8035, 84242, 55304, 52999, or 21999 substrates, to screen for drugs or compounds which modulate 8035, 84242, 55304, 52999, or 21999 activity, as well as to treat disorders characterized by insufficient or excessive production of 8035, 84242, 55304, 52999, or 21999 protein or production of 8035, 84242, 55304, 52999, or 21999 protein forms which have decreased, aberrant or unwanted activity compared to 8035, 84242, 55304, 52999, or 21999 wild-type protein. In the case of 8035 and 84242, such disorders include those characterized by aberrant cellular proliferative and/or differentiative disorders. In the case of 55304, such disorders include those characterized by aberrant protein proteolysis or maturation or aberrant, e.g. hyperproliferative, cell growth. In the case of 52999, such disorders include those characterized by aberrant protein processing or protein degradation. In the case of 21999, such disorders include those characterized by aberrant cellular metabolism or aberrant growth, e.g., hyperproliferative, cell growth. Moreover, the anti-8035, anti-84242, anti-55304, anti-52999, or anti-21999 antibodies of the invention can be used to detect and isolate 8035, 84242, 55304, 52999, or 21999 proteins, regulate the bioavailability of 8035, 84242, 55304, 52999, or 21999 proteins, and modulate 8035, 84242, 55304, 52999, or 21999 activity, respectively.

A method of evaluating a compound for the ability to interact with, e.g., bind, a subject 8035, 84242, 55304, 52999, or 21999 polypeptide is provided. The method includes: contacting the compound with the subject 8035, 84242, 55304, 52999, or 21999 polypeptide; and evaluating ability of the compound to interact with, e.g., to bind or form a complex with the subject 8035, 84242, 55304, 52999, or 21999 polypeptide. This method can be performed in vitro, e.g., in a cell free system, or in vivo, e.g., in a two-hybrid interaction trap assay. This method can be used to identify naturally occurring molecules which interact with subject 8035, 84242, 55304, 52999, or 21999 polypeptide. It can also be used to find natural or synthetic inhibitors of subject 8035, 84242, 55304, 52999, or 21999 polypeptide. Screening methods are discussed in more detail below.

Screening Assays:

The invention provides methods (also referred to herein as “screening assays”) for identifying modulators, i.e., candidate or test compounds or agents (e.g., proteins, peptides, peptidomimetics, peptoids, small molecules or other drugs) which bind to 8035, 84242, 55304, 52999, or 21999 proteins, have a stimulatory or inhibitory effect on, for example, 8035, 84242, 55304, 52999, or 21999 expression or 8035, 84242, 55304, 52999, or 21999 activity, or have a stimulatory or inhibitory effect on, for example, the expression or activity of an 8035, 84242, 55304, 52999, or 21999 substrate. Compounds thus identified can be used to modulate the activity of target gene products (e.g., 8035, 84242, 55304, 52999, or 21999 genes) in a therapeutic protocol, to elaborate the biological function of the target gene product, or to identify compounds that disrupt normal target gene interactions.

In one embodiment, the invention provides assays for screening candidate or test compounds which are substrates of an 8035, 84242, 55304, 52999, or 21999 protein or polypeptide or a biologically active portion thereof. In another embodiment, the invention provides assays for screening candidate or test compounds which bind to or modulate the activity of an 8035, 84242, 55304, 52999, or 21999 protein or polypeptide or a biologically active portion thereof.

The test compounds of the present invention can be obtained using any of the numerous approaches in combinatorial library methods known in the art, including: biological libraries; peptoid libraries [libraries of molecules having the functionalities of peptides, but with a novel, non-peptide backbone which are resistant to enzymatic degradation but which nevertheless remain bioactive] (see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 3 7:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria or spores (Ladner, U.S. Pat. No. 5,223,409), plasmids (Cull et al. (1992) Proc. Natl. Acad. Sci. USA 89:1865-1869) or on phage (Scott and Smith (1990) Science 249:386-390); (Devlin (1990) Science 249:404 -406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses an 8035, 84242, 55304, 52999, or 21999 protein or biologically active portion thereof is contacted with a test compound, and the ability of the test compound to modulate 8035, 84242, 55304, 52999, or 21999 activity is determined. Determining the ability of the test compound to modulate 8035 or 84242 activity can be accomplished by monitoring, for example, RING finger E3 ubiquitin ligase protein activity. Determining the ability of the test compound to modulate 55304 activity can be accomplished by monitoring, for example, aminopeptidase activity. Determining the ability of the test compound to modulate 52999 activity can be accomplished by monitoring, for example, polypeptide hydrolytic activity. Determining the ability of the test compound to modulate 21999 activity can be accomplished by monitoring, for example, ADP-moiety transferase activity. The cell, for example, can be of mammalian origin, e.g., human. Cell homogenates, or fractions, preferably membrane containing fractions, can also be tested.

The ability of the test compound to modulate 8035, 84242, 55304, 52999, or 21999 binding to a compound, e.g., an 8035, 84242, 55304, 52999, or 21999 substrate, or to bind to 8035, 84242, 55304, 52999, or 21999 can also be evaluated. This can be accomplished, for example, by coupling the compound, e.g., the substrate, with a radioisotope or enzymatic label such that binding of the compound, e.g., the substrate, to 8035, 84242, 55304, 52999, or 21999 can be determined by detecting the labeled compound, e.g., substrate, in a complex. Alternatively, 8035, 84242, 55304, 52999, or 21999 could be coupled with a radioisotope or enzymatic label to monitor the ability of a test compound to modulate 8035, 84242, 55304, 52999, or 21999 binding to an 8035, 84242, 55304, 52999, or 21999 substrate in a complex. For example, compounds (e.g., 8035, 84242, 55304, 52999, or 21999 substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., an 8035, 84242, 55304, 52999, or 21999 substrate) to interact with 8035, 84242, 55304, 52999, or 21999 with or without the labeling of any of the interactants can be evaluated. For example, a microphysiometer can be used to detect the interaction of a compound with 8035, 84242, 55304, 52999, or 21999 without the labeling of either the compound or the 8035, 84242, 55304, 52999, or 21999. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 8035, 84242, 55304, 52999, or 21999.

In yet another embodiment, a cell-free assay is provided in which an 8035, 84242, 55304, 52999, or 21999 protein or biologically active portion thereof is contacted with a test compound and the ability of the test compound to bind to the 8035, 84242, 55304, 52999, or 21999 protein or biologically active portion thereof is evaluated. Preferred biologically active portions of the 8035, 84242, 55304, 52999, or 21999 proteins to be used in assays of the present invention include fragments which participate in interactions with non-8035, 84242, 55304, 52999, or 21999 molecules, e.g., fragments with high surface probability scores.

Soluble and/or membrane-bound forms of isolated proteins (e.g., 8035, 84242, 55304, 52999, or 21999 proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

In one embodiment related to 8035 and 84242, assays are performed where the ability of an agent to block RING finger protein E3 ubiquitin ligase activity within a cell is evaluated. In another embodiment involving 55304, assays are performed where the ability of an agent to block aminopeptidase activity within a cell is evaluated. In yet another embodiment related to 52999, assays are performed where the ability of an agent to block metallopeptidase activity within a cell is evaluated. In another embodiment related to 21999, an assay is a cell-based assay in which a cell which expresses a ADP-ribosyltransferase protein or biologically active portion thereof is contacted with a test compound, and the ability of the test compound to modulate ADP-ribosyltransferase activity is determined. Determining the ability of the test compound to modulate ADP-ribosyltransferase activity can be accomplished by monitoring, for example, ADP-moiety transferase activity. The cell, for example, can be of mammalian origin, e.g., human. Cell homogenates, or fractions, preferably membrane containing fractions, can also be tested.

The interaction between two molecules can also be detected, e.g., using fluorescence energy transfer (FET) (see, for example, Lakowicz et al. U.S. Pat. No. 5,631,169; Stavrianopoulos, et al. U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the 8035, 84242, 55304, 52999, or 21999 protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either 8035, 84242, 55304, 52999, or 21999, an anti-8035, anti-84242, anti-55304, anti-52999, or anti-21999 antibody, or its target molecule, respectively, to facilitate separation of complexed from uncomplexed forms of one or both of the proteins, as well as to accommodate automation of the assay. Binding of a test compound to an 8035, 84242, 55304, 52999, or 21999 protein, or interaction of an 8035, 84242, 55304, 52999, or 21999 protein with a target molecule, respectively, in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/8035, glutathione-S-transferase/84242, glutathione-S-transferase/55304, glutathione-S-transferase/52999, or glutathione-S-transferase/21999 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or 8035, 84242, 55304, 52999, or 21999 protein, respectively, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of 8035, 84242, 55304, 52999, or 21999 binding or activity determined using standard techniques.

Other techniques for immobilizing either an 8035, 84242, 55304, 52999, or 21999 protein or a target molecule on matrices include using conjugation of biotin and streptavidin. Biotinylated 8035, 84242, 55304, 52999, or 21999 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with 8035, 84242, 55304, 52999, or 21999 protein or target molecules but which do not interfere with binding of the 8035, 84242, 55304, 52999, or 21999 protein, respectively, to its target molecule. Such antibodies can be derivatized to the wells of the plate, and unbound target or 8035, 84242, 55304, 52999, or 21999 protein trapped in the wells by antibody conjugation. Methods for detecting such complexes, in addition to those described above for the GST-immobilized complexes, include immunodetection of complexes using antibodies reactive with the 8035, 84242, 55304, 52999, or 21999 protein or target molecule, as well as enzyme-linked assays which rely on detecting an enzymatic activity associated with the 8035, 84242, 55304, 52999, or 21999 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P. (August 1993) Trends Biochem Sci 18(8):284-7); chromatography (gel filtration chromatography, ion-exchange chromatography); electrophoresis (see, e.g., Ausubel, F. et al. eds. Current Protocols in Molecular Biology 1999, J. Wiley: New York.); and immunoprecipitation (see, for example, Ausubel, F. et al. eds. Current Protocols in Molecular Biology 1999, J. Wiley: New York). Such resins and chromatographic techniques are known to one skilled in the art (see, e.g., Heegaard, N. H. (1998 Winter) J Mol. Recognit.11 (1-6):141-8; Hage, D. S., and Tweed, S.A. (October 1997) J. Chromatogr. B Biomed. Sci. Appl.699(1-2):499-525). Further, fluorescence energy transfer may also be conveniently utilized, as described herein, to detect binding without further purification of the complex from solution.

In a preferred embodiment, the assay includes contacting the 8035, 84242, 55304, 52999, or 21999 protein or biologically active portion thereof with a known compound which binds 8035, 84242, 55304, 52999, or 21999, respectively, to form an assay mixture, contacting the assay mixture with a test compound, and determining the ability of the test compound to interact with an 8035, 84242, 55304, 52999, or 21999 protein, wherein determining the ability of the test compound to interact with an 8035, 84242, 55304, 52999, or 21999 protein includes determining the ability of the test compound to preferentially bind to 8035, 84242, 55304, 52999, or 21999 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the 8035, 84242, 55304, 52999, or 21999 genes herein identified. In an alternative embodiment, the invention provides methods for determining the ability of the test compound to modulate the activity of an 8035, 84242, 55304, 52999, or 21999 protein through modulation of the activity of a downstream effector of an 8035, 84242, 55304, 52999, or 21999 target molecule, respectively. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), e.g., a substrate, a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the 8035, 84242, 55304, 52999, or 21999 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent W094/10300), to identify other proteins, which bind to or interact with 8035, 84242, 55304, 52999, or 21999 (“8035, 84242, 55304, 52999, or 21999-binding proteins” or “8035, 84242, 55304, 52999, or 21999-bp”) and are involved in 8035, 84242, 55304, 52999, or 21999 activity, respectively. Such8035, 84242, 55304, 52999, or 21999-bps can be activators or inhibitors of signals by the 8035, 84242, 55304, 52999, or 21999 proteins or 8035, 84242, 55304, 52999, or 21999 targets, respectively, as, for example, downstream elements of an 8035, 84242, 55304, 52999, or 21999-mediated signaling pathway.

The two-hybrid system is based on the modular nature of most transcription factors, which consist of separable DNA-binding and activation domains. Briefly, the assay utilizes two different DNA constructs. In one construct, the gene that codes for a 8035, 84242, 55304, 52999, or 21999 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: 8035, 84242, 55304, 52999, or 21999 protein can be fused to the activator domain.) If the “bait” and the “prey” proteins are able to interact, in vivo, forming an 8035, 84242, 55304, 52999, or 21999-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 8035, 84242, 55304, 52999, or 21999 protein.

In another embodiment, modulators of 8035, 84242, 55304, 52999, or 21999 expression are identified. For example, a cell or cell free mixture is contacted with a candidate compound and the expression of 8035, 84242, 55304, 52999, or 21999 mRNA or protein evaluated relative to the level of expression of 8035, 84242, 55304, 52999, or 21999 mRNA or protein in the absence of the candidate compound. When expression of 8035, 84242, 55304, 52999, or 21999 mRNA or protein is greater in the presence of the candidate compound than in its absence, the candidate compound is identified as a stimulator of 8035, 84242, 55304, 52999, or 21999 mRNA or protein expression, respectively. Alternatively, when expression of 8035, 84242, 55304, 52999, or 21999 mRNA or protein is less (statistically significantly less) in the presence of the candidate compound than in its absence, the candidate compound is identified as an inhibitor of 8035, 84242, 55304, 52999, or 21999 mRNA or protein expression, respectively. The level of 8035, 84242, 55304, 52999, or 21999 mRNA or protein expression can be determined by methods described herein for detecting 8035, 84242, 55304, 52999, or 21999 mRNA or protein, respectively.

In another aspect, the invention pertains to a combination of two or more of the assays described herein. For example, a modulating agent can be identified using a cell-based or a cell free assay, and the ability of the agent to modulate the activity of an 8035, 84242, 55304, 52999, or 21999 protein can be confirmed in vivo, e.g., in an animal.

This invention further pertains to novel agents identified by the above-described screening assays. Accordingly, it is within the scope of this invention to further use an agent identified as described herein (e.g., an 8035, 84242, 55304, 52999, or 21999 modulating agent, an antisense 8035, 84242, 55304, 52999, or 21999 nucleic acid molecule, an 8035, 84242, 55304, 52999, or 21999-specific antibody, or a 8035, 84242, 55304, 52999, or 21999-binding partner) in an appropriate animal model to determine the efficacy, toxicity, side effects, or mechanism of action, of treatment with such an agent. Furthermore, novel agents identified by the above-described screening assays can be used for treatments as described herein.

Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate 8035, 84242, 55304, 52999, or 21999 with a disease; (ii) identify an individual from a minute biological sample (tissue typing); and (iii) aid in forensic identification of a biological sample. These applications are described in the subsections below.

Chromosome Mapping

The 8035, 84242, 55304, 52999, or 21999 nucleotide sequences or portions thereof can be used to map the location of the 8035, 84242, 55304, 52999, or 21999 genes, respectively, on a chromosome. This process is called chromosome mapping. Chromosome mapping is useful in correlating the 8035, 84242, 55304, 52999, or 21999 sequences with genes associated with disease.

Briefly, 8035, 84242, 55304, 52999, or 21999 genes can be mapped to chromosomes by preparing PCR primers (preferably 15-25 bp in length) from the 8035, 84242, 55304, 52999, or 21999 nucleotide sequences. These primers can then be used for PCR screening of somatic cell hybrids containing individual human chromosomes. Only those hybrids containing the human gene corresponding to the 8035, 84242, 55304, 52999, or 21999 sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes. (D'Eustachio P. et al. (1983) Science 220:919-924).

Other mapping strategies e.g., in situ hybridization (described in Fan, Y. et al. (1990) Proc. Natl. Acad. Sci. USA 87:6223-27), pre-screening with labeled flow-sorted chromosomes, and pre-selection by hybridization to chromosome specific cDNA libraries can be used to map 8035, 84242, 55304, 52999, or 21999 to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al. Human Chromosomes: A Manual of basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature 325:783-787.

Moreover, differences in the DNA sequences between individuals affected and unaffected with a disease associated with the 8035, 84242, 55304, 52999, or 21999 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

8035, 84242, 55304, 52999, or 21999 sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the 8035, 84242, 55304, 52999, or 21999 nucleotide sequences described herein can be used to prepare two PCR primers from the 5′ and 3′ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO: 1, SEQ ID NO: 5, SEQ ID NO: 9, SEQ ID NO: 12, and SEQ ID NO: 19 can provide positive individual identification with a panel of perhaps 10 to 1,000 primers which each yield a noncoding amplified sequence of 100 bases. If predicted coding sequences, such as those in SEQ ID NO: 3, SEQ ID NO: 7, SEQ ID NO: 11, SEQ ID NO: 14, or SEQ ID NO: 21 are used, a more appropriate number of primers for positive individual identification would be 500-2,000.

If a panel of reagents from 8035, 84242, 55304, 52999, or 21999 nucleotide sequences described herein is used to generate a unique identification database for an individual, those same reagents can later be used to identify tissue from that individual. Using the unique identification database, positive identification of the individual, living or dead, can be made from extremely small tissue samples.

Use of Partial 8035, 84242, 55304, 52999, or 21999 Sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO: 1, SEQ ID NO: 5, SEQ ID NO: 9, SEQ ID NO: 12, or SEQ ID NO: 19 (e.g., fragments derived from the noncoding regions of SEQ ID NO: 1, SEQ ID NO: 5, SEQ ID NO: 9, SEQ ID NO: 12, or SEQ ID NO: 19 having a length of at least 20 bases, preferably at least 30 bases) are particularly appropriate for this use.

The 8035, 84242, 55304, 52999, or 21999 nucleotide sequences described herein can further be used to provide polynucleotide reagents, e.g., labeled or labelable probes which can be used in, for example, an in situ hybridization technique, to identify a specific tissue, e.g., a tissue containing RING finger E3 ubiquitin ligase protein activity (8035 and 84242), aminopeptidase activity (55304), metallopreptidase activity (52999), and ADP-ribosyltransferase activity (21999). This can be very useful in cases where a forensic pathologist is presented with a tissue of unknown origin. Panels of such 8035, 84242, 55304, 52999, or 21999 probes can be used to identify tissue by species and/or by organ type.

In a similar fashion, these reagents, e.g., 8035, 84242, 55304, 52999, or 21999 primers or probes can be used to screen tissue culture for contamination (i.e. screen for the presence of a mixture of different types of cells in a culture).

Predictive Medicine

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes 8035, 84242, 55304, 52999, or 21999.

Such disorders include, e.g., a disorder associated with the misexpression of 8035 or 84242, those disorders resulting from aberrant cellular proliferation and/or differentiation including diseases such as cancer, acute promyelocytic leukemia (APL), VHL disease, and systemic lupus erythematosus. In addition, RING finger protein family members have been shown to contribute to the pathogenesis of certain viral diseases including those caused by HSV and HIV. Other such disorders include, e.g., a disorder associated with the misexpression of 55304, 52999, 21999, or lipid metabolism related disorder.

The method includes one or more of the following:

detecting, in a tissue of the subject, the presence or absence of a mutation which affects the expression of the 8035, 84242, 55304, 52999, or 21999 gene, or detecting the presence or absence of a mutation in a region which controls the expression of the gene, e.g., a mutation in the 5′ control region;

detecting, in a tissue of the subject, the presence or absence of a mutation which alters the structure of the 8035, 84242, 55304, 52999, or 21999 gene;

detecting, in a tissue of the subject, the misexpression of the 8035, 84242, 55304, 52999, or 21999 gene, at the mRNA level, e.g., detecting a non-wild type level of an mRNA;

detecting, in a tissue of the subject, the misexpression of the gene, at the protein level, e.g., detecting a non-wild type level of an 8035, 84242, 55304, 52999, or 21999 polypeptide.

In preferred embodiments the method includes: ascertaining the existence of at least one of: a deletion of one or more nucleotides from the 8035, 84242, 55304, 52999, or 21999 gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO: 1, SEQ ID NO: 5, SEQ ID NO: 9, SEQ ID NO: 12, SEQ ID NO: 19, naturally occurring mutants thereof or 5′ or 3′ flanking sequences naturally associated with the 8035, 84242, 55304, 52999, or 21999 gene, respectively; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of the 8035, 84242, 55304, 52999, or 21999 gene; the presence of a non-wild type splicing pattern of a messenger RNA transcript of the gene; or a non-wild type level of 8035, 84242,55304,52999, or 21999.

Methods of the invention can be used prenatally or to determine if a subject's offspring will be at risk for a disorder.

In preferred embodiments the method includes determining the structure of an 8035, 84242, 55304, 52999, or 21999 gene, an abnormal structure being indicative of risk for the disorder.

In preferred embodiments the method includes contacting a sample form the subject with an antibody to the 8035, 84242, 55304, 52999, or 21999 protein or a nucleic acid, which hybridizes specifically with the gene. These and other embodiments are discussed below.

Diagnostic and Prognostic Assays

The presence, level, or absence of 8035, 84242, 55304, 52999, or 21999 protein or nucleic acid in a biological sample can be evaluated by obtaining a biological sample from a test subject and contacting the biological sample with a compound or an agent capable of detecting 8035, 84242, 55304, 52999, or 21999 protein or nucleic acid (e.g., mRNA, genomic DNA) that encodes 8035, 84242, 55304, 52999, or 21999 protein such that the presence of 8035, 84242, 55304, 52999, or 21999 protein or nucleic acid is detected in the biological sample. The term “biological sample” includes tissues, cells and biological fluids isolated from a subject, as well as tissues, cells and fluids present within a subject. The level of expression of the 8035, 84242, 55304, 52999, or 21999 gene can be measured in a number of ways, including, but not limited to: measuring the mRNA encoded by the 8035, 84242, 55304, 52999, or 21999 genes; measuring the amount of protein encoded by the 8035, 84242, 55304, 52999, or 21999 genes; or measuring the activity of the protein encoded by the 8035, 84242, 55304, 52999, or 21999 genes.

The level of mRNA corresponding to the 8035, 84242, 55304, 52999, or 21999 gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length 8035, 84242, 55304, 52999, or 21999 nucleic acid, such as the nucleic acid of SEQ ID NO: 1, SEQ ID NO: 5, SEQ ID NO: 9, SEQ ID NO: 12, SEQ ID NO: 19, or the DNA insert of the plasmid deposited with ATCC as Accession Number ______, ______, ______, ______, or ______, or a portion thereof, such as an oligonucleotide of at least 7, 15, 30, 50, 100, 250 or 500 nucleotides in length and sufficient to specifically hybridize under stringent conditions to 8035, 84242, 55304, 52999, or 21999 mRNA or genomic DNA, respectively. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array. A skilled artisan can adapt known mRNA detection methods for use in detecting the level of mRNA encoded by the 8035, 84242, 55304, 52999, or 21999 genes.

The level of mRNA in a sample that is encoded by one of 8035, 84242, 55304, 52999, or 21999 can be evaluated with nucleic acid amplification, e.g., by rtPCR (Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al. U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the 8035, 84242, 55304, 52999, or 21999 gene being analyzed.

In another embodiment, the methods further contacting a control sample with a compound or agent capable of detecting 8035, 84242, 55304, 52999, or 21999 mRNA, or genomic DNA, and comparing the presence of 8035, 84242, 55304, 52999, or 21999 mRNA or genomic DNA in the control sample with the presence of 8035, 84242, 55304, 52999, or 21999 mRNA or genomic DNA, respectively, in the test sample.

A variety of methods can be used to determine the level of protein encoded by 8035, 84242, 55304, 52999, or 21999. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect 8035, 84242, 55304, 52999, or 21999 protein in a biological sample in vitro as well as in vivo. In vitro techniques for detection of 8035, 84242, 55304, 52999, or 21999 protein include enzyme linked immunosorbent assays (ELISAs), immunoprecipitations, immunofluorescence, enzyme immunoassay (EIA), radioimmunoassay (RIA), and Western blot analysis. In vivo techniques for detection of 8035, 84242, 55304, 52999, or 21999 protein include introducing into a subject a labeled anti-8035, anti-84242, anti-55304, anti-52999, or anti-21999 antibody. For example, the antibody can be labeled with a radioactive marker whose presence and location in a subject can be detected by standard imaging techniques.

In another embodiment, the methods further include contacting the control sample with a compound or agent capable of detecting 8035, 84242, 55304, 52999, or 21999 protein, and comparing the presence of 8035, 84242, 55304, 52999, or 21999 protein in the control sample with the presence of 8035, 84242, 55304, 52999, or 21999 protein, respectively, in the test sample.

The invention also includes kits for detecting the presence of 8035, 84242, 55304, 52999, or 21999 in a biological sample. For example, the kit can include a compound or agent capable of detecting 8035, 84242, 55304, 52999, or 21999 protein or mRNA in a biological sample; and a standard. The compound or agent can be packaged in a suitable container. The kit can further comprise instructions for using the kit to detect 8035, 84242, 55304, 52999, or 21999 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein-stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted 8035, 84242, 55304, 52999, or 21999 expression or activity. As used herein, the term “unwanted” includes an unwanted phenomenon involved in a biological response such as, for example, in the case of 8035 and 84242, deregulated cell proliferation and/or differentiation, in the case of 55304 deregulated cell proliferation or hypertension, in the case of 52999, inflammation or deregulated cell proliferation, or in the case of 21999, deregulated cell proliferation or depressed cellular metabolism.

In one embodiment, a disease or disorder associated with aberrant or unwanted 8035, 84242, 55304, 52999, or 21999 expression or activity is identified. A test sample is obtained from a subject and 8035, 84242, 55304, 52999, or 21999 protein or nucleic acid (e.g., mRNA or genomic DNA) is evaluated, wherein the level, e.g., the presence or absence, of 8035, 84242, 55304, 52999, or 21999 protein or nucleic acid is diagnostic for a subject having or at risk of developing a disease or disorder associated with aberrant or unwanted 8035, 84242, 55304, 52999, or 21999 expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 8035, 84242, 55304, 52999, or 21999 expression or activity. For example, such methods can be used to determine whether a subject can be effectively treated with an agent for a cellular growth related disorder.

The methods of the invention can also be used to detect genetic alterations in a 8035, 84242, 55304, 52999, or 21999 gene, thereby determining if a subject with the altered gene is at risk for a disorder characterized by misregulation in 8035, 84242, 55304, 52999, or 21999 protein activity or nucleic acid expression, such as a cellular growth related disorder. In preferred embodiments, the methods include detecting, in a sample from the subject, the presence or absence of a genetic alteration characterized by at least one of an alteration affecting the integrity of a gene encoding an 8035, 84242, 55304, 52999, or 21999-protein, or the misexpression of the 8035, 84242, 55304, 52999, or 21999 gene. For example, such genetic alterations can be detected by ascertaining the existence of at least one of 1) a deletion of one or more nucleotides from an 8035, 84242, 55304, 52999, or 21999 gene; 2) an addition of one or more nucleotides to an 8035, 84242, 55304, 52999, or 21999 gene; 3) a substitution of one or more nucleotides of an 8035, 84242, 55304, 52999, or 21999 gene, 4) a chromosomal rearrangement of an 8035, 84242, 55304, 52999, or 21999 gene; 5) an alteration in the level of a messenger RNA transcript of an 8035, 84242, 55304, 52999, or 21999 gene, 6) aberrant modification of an8035, 84242, 55304, 52999, or 21999 gene, such as of the methylation pattern of the genomic DNA, 7) the presence of a non-wild type splicing pattern of a messenger RNA transcript of an 8035, 84242, 55304, 52999, or 21999 gene, 8) a non-wild type level of an 8035, 84242, 55304, 52999, or 21999-protein, 9) allelic loss of an 8035, 84242, 55304, 52999, or 21999 gene, and 10) inappropriate post-translational modification of an 8035, 84242, 55304, 52999, or 21999-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the 8035, 84242, 55304, 52999, or 21999-gene. This method can include the steps of collecting a sample of cells from a subject, isolating nucleic acid (e.g., genomic, mRNA or both) from the sample, contacting the nucleic acid sample with one or more primers which specifically hybridize to an 8035, 84242, 55304, 52999, or 21999 gene under conditions such that hybridization and amplification of the 8035, 84242, 55304, 52999, or 21999-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86: 1173-1177), Q-Beta Replicase (Lizardi, P. M. et al. (1988) Bio-Technology 6:1197), or other nucleic acid amplification methods, followed by the detection of the amplified molecules using techniques known to those of skill in the art.

In another embodiment, mutations in an 8035, 84242, 55304, 52999, or 21999 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 8035, 84242, 55304, 52999, or 21999 can be identified by hybridizing a sample and control nucleic acids, e.g., DNA or RNA, two-dimensional arrays, e.g., chip based arrays. Such arrays include a plurality of addresses, each of which is positionally distinguishable from the other. A different probe is located at each address of the plurality. The arrays can have a high density of addresses, e.g., can contain hundreds or thousands of oligonucleotides probes (Cronin, M. T. et al. (1996) Human Mutation 7: 244-255; Kozal, M. J. et al. (1996) Nature Medicine 2:753-759). For example, genetic mutations in 8035, 84242, 55304, 52999, or 21999 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 8035, 84242, 55304, 52999, or 21999 gene and detect mutations by comparing the sequence of the sample 8035, 84242, 55304, 52999, or 21999 with the corresponding wild-type (control) sequence. Automated sequencing procedures can be utilized when performing the diagnostic assays (Naeve et al.(1995) Biotechniques 19:448-453), including sequencing by mass spectrometry.

Other methods for detecting mutations in the 8035, 84242, 55304, 52999, or 21999 gene include methods in which protection from cleavage agents is used to detect mismatched bases in RNA/RNA or RNA/DNA heteroduplexes (Myers et al. (1985) Science 230:1242-1246; Cotton et al. (1988) Proc. Natl. Acad. Sci. USA 85:4397-4401; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in 8035, 84242, 55304, 52999, or 21999 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in 8035, 84242, 55304, 52999, or 21999 genes. For example, single strand conformation polymorphism (SSCP) may be used to detect differences in electrophoretic mobility between mutant and wild type nucleic acids (Orita et al. (1989) Proc. Natl. Acad. Sci. USA: 86:2766-2770, see also Cotton (1993) Mutat. Res. 285:125 -144; and Hayashi (1992) Genet. Anal. Tech. Appl. 9:73-79). Single-stranded DNA fragments of sample and control 8035, 84242, 55304, 52999, or 21999 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495-498). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl. Acad. Sci. USA 86:6230).

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1-7). It is anticipated that in certain embodiments amplification may also be performed using Taq ligase for amplification (Barany (1991) Proc. Natl. Acad. Sci USA 88:189-193). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an 8035, 84242, 55304, 52999, or 21999 gene.

Use of 8035. 84242 55304. 52999. or 21999 Molecules as Surrogate Markers

The 8035, 84242, 55304, 52999, or 21999 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 8035, 84242, 55304, 52999, or 21999 molecules of the invention may be detected, and may be correlated with one or more biological states in vivo. For example, the 8035, 84242, 55304, 52999, or 21999 molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 8035, 84242, 55304, 52999, or 21999 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., an 8035, 84242, 55304, 52999, or 21999 marker) transcription or expression, the amplified marker may be in a quantity which is more readily detectable than the drug itself. Also, the marker may be more easily detected due to the nature of the marker itself; for example, using the methods described herein, anti-8035, anti-84242, anti-55304, anti-52999, or anti-21999 antibodies may be employed in an immune-based detection system for an 8035, 84242, 55304, 52999, or 21999 protein marker, respectively, or 8035, 84242, 55304, 52999, or 21999-specific radiolabeled probes may be used to detect an 8035, 84242, 55304, 52999, or 21999 mRNA marker, respectively. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90:229-238; Schentag (1999) Am. J. Health-Syst. Pharm. 56 Suppl.3:S21-S24; and Nicolau (1999) Am, J. Health-Syst. Pharm. 56 Suppl.3:S16-S20.

The 8035, 84242, 55304, 52999, or 21999 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12): 1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., 8035, 84242, 55304, 52999, or 21999 protein or RNA) for specific tumor markers in a subject, a drug or course of treatment may be selected that is optimized for the treatment of the specific tumor likely to be present in the subject. Similarly, the presence or absence of a specific sequence mutation in 8035, 84242, 55304, 52999, or 21999 DNA may correlate 8035, 84242, 55304, 52999, or 21999 drug response, respectively. The use of pharmacogenomic markers therefore permits the application of the most appropriate treatment for each subject without having to administer the therapy.

Pharmaceutical Compositions

The nucleic acid and polypeptides, fragments thereof, as well as anti-8035, anti-84242, anti-55304, anti-52999, or anti-21999 antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents which modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated.

When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of Treatment:

The present invention provides for both prophylactic and therapeutic methods of treating a subject at risk of (or susceptible to) a disorder or having a disorder associated with aberrant or unwanted 8035, 84242, 55304, 52999, or 21999 expression or activity. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the 8035, 84242, 55304, 52999, or 21999 molecules of the present invention or 8035, 84242, 55304, 52999, or 21999 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with an aberrant or unwanted 8035, 84242, 55304, 52999, or 21999 expression or activity, by administering to the subject an 8035, 84242, 55304, 52999, or 21999 agent which modulates 8035, 84242, 55304, 52999, or 21999 expression or at least one 8035, 84242, 55304, 52999, or 21999 activity, respectively. Subjects at risk for a disease which is caused or contributed to by aberrant or unwanted 8035, 84242, 55304, 52999, or 21999 expression or activity can be identified by, for example, any or a combination of diagnostic or prognostic assays as described herein. Administration of a prophylactic agent can occur prior to the manifestation of symptoms characteristic of the 8035, 84242, 55304, 52999, or 21999 aberrance, such that a disease or disorder is prevented or, alternatively, delayed in its progression. Depending on the type of 8035, 84242, 55304, 52999, or 21999 aberrance, for example, an 8035, 84242, 55304, 52999, or 21999 agonist, or an 8035, 84242, 55304, 52999, or 21999 antagonist agent can be used for treating the subject. The appropriate agent can be determined based on screening assays described herein.

It is possible that some 8035, 84242, 55304, 52999, or 21999 disorders can be caused, at least in part, by an abnormal level of gene product, or by the presence of a gene product exhibiting abnormal activity. As such, the reduction in the level and/or activity of such gene products would bring about the amelioration of disorder symptoms.

As discussed, successful treatment of 8035, 84242, 55304, 52999, or 21999 disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of 8035, 84242, 55304, 52999, or 21999 disorders. Such molecules can include, but are not limited to peptides, phosphopeptides, small organic or inorganic molecules, or antibodies (including, for example, polyclonal, monoclonal, humanized, anti-idiotypic, chimeric or single chain antibodies, and FAb, F(ab′)₂ and FAb expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules may be utilized in treating or preventing a disease characterized by 8035, 84242, 55304, 52999, or 21999 expression is through the use of aptamer molecules specific for 8035, 84242, 55304, 52999, or 21999 protein, respectively. Aptamers are nucleic acid molecules having a tertiary structure which permits them to specifically bind to protein ligands (see, e.g., Osborne, et al. (1997) Curr. Opin. Chem. Biol. 1 (1):5-9; and Patel, D.J. (1997 Jun) Curr. Opin. Chem. Biol 1(1):32-46). Since nucleic acid molecules may in many cases be more conveniently introduced into target cells than therapeutic protein molecules may be, aptamers offer a method by which 8035, 84242, 55304, 52999, or 21999 protein activity may be specifically decreased without the introduction of drugs or other molecules which may have pluripotent effects.

Antibodies can be generated that are both specific for target gene product and that reduce target gene product activity. Such antibodies may, therefore, be administered in instances whereby negative modulatory techniques are appropriate for the treatment of 8035, 84242, 55304, 52999, or 21999 disorders. For a description of antibodies, see the Antibody section above.

In circumstances wherein injection of an animal or a human subject with an 8035, 84242, 55304, 52999, or 21999 protein or epitope for stimulating antibody production is harmful to the subject, it is possible to generate an immune response against 8035, 84242, 55304, 52999, or 21999 through the use of anti-idiotypic antibodies (see, for example, Herlyn, D. (1999) Ann. Med. 31(1):66-78; and Bhattacharya-Chatterjee, M., and Foon, K. A. (1998) Cancer Treat. Res. 94:51-68). If an anti-idiotypic antibody is introduced into a mammal or human subject, it should stimulate the production of anti-anti-idiotypic antibodies, which should be specific to the 8035, 84242, 55304, 52999, or 21999 protein. Vaccines directed to a disease characterized by 8035, 84242, 55304, 52999, or 21999 expression may also be generated in this fashion.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993) Proc. Natl. Acad. Sci. USA 90:7889-7893).

The identified compounds that inhibit target gene expression, synthesis and/or activity can be administered to a patient at therapeutically effective doses to prevent, treat or ameliorate 8035, 84242, 55304, 52999, or 21999 disorders. A therapeutically effective dose refers to that amount of the compound sufficient to result in amelioration of symptoms of the disorders.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.Compounds that exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate 8035, 84242, 55304, 52999, or 21999 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell, R. J. et al. (1996) Current Opinion in Biotechnology 7:89-94 and in Shea, K. J. (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, G. et al (1993) Nature 361:645-647. Through the use of isotope-labeling, the “free” concentration of compound which modulates the expression or activity of 8035, 84242, 55304, 52999, or 21999 can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. A rudimentary example of such a “biosensor” is discussed in Kriz, D. et al. (1995) Analytical Chemistry 67:2142-2144.

Another aspect of the invention pertains to methods of modulating 8035, 84242, 55304, 52999, or 21999 expression or activity for therapeutic purposes. Accordingly, in an exemplary embodiment, the modulatory method of the invention involves contacting a cell with an 8035, 84242, 55304, 52999, or 21999 agent that modulates one or more of the activities of 8035, 84242, 55304, 52999, or 21999 protein activity, respectively, associated with the cell. An agent that modulates 8035, 84242, 55304, 52999, or 21999 protein activity can be an agent as described herein, such as a nucleic acid or a protein, a naturally-occurring target molecule of an 8035, 84242, 55304, 52999, or 21999 protein (e.g., an 8035, 84242, 55304, 52999, or 21999 substrate or binding partner), an 8035, 84242, 55304, 52999, or 21999 antibody, an 8035, 84242, 55304, 52999, or 21999 agonist or antagonist, a peptidomimetic of an 8035, 84242, 55304, 52999, or 21999 agonist or antagonist, or other small molecule.

In one embodiment, the agent stimulates one or more 8035, 84242, 55304, 52999, or 21999 activities. Examples of such stimulatory agents include active 8035, 84242, 55304, 52999, or 21999 protein and a nucleic acid molecule encoding 8035, 84242, 55304, 52999, or 21999, respectively. In another embodiment, the agent inhibits one or more 8035, 84242, 55304, 52999, or 21999 activities. Examples of such inhibitory agents include antisense 8035, 84242, 55304, 52999, or 21999 nucleic acid molecules, anti-8035, anti-84242, anti-55304, anti-52999, or anti-21999 antibodies, and 8035, 84242, 55304, 52999, or 21999 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of an 8035, 84242, 55304, 52999, or 21999 protein or nucleic acid molecule. In one embodiment, the method involves administering an agent (e.g., an agent identified by a screening assay described herein), or combination of agents that modulates (e.g., upregulates or downregulates) 8035, 84242, 55304, 52999, or 21999 expression or activity. In another embodiment, the method involves administering an 8035, 84242, 55304, 52999, or 21999 protein or nucleic acid molecule as therapy to compensate for reduced, aberrant, or unwanted 8035, 84242, 55304, 52999, or 21999 expression or activity, respectively.

Stimulation of 8035, 84242, 55304, 52999, or 21999 activity is desirable in situations in which 8035, 84242, 55304, 52999, or 21999 is abnormally downregulated and/or in which increased 8035, 84242, 55304, 52999, or 21999 activity is likely to have a beneficial effect. For example, stimulation of 8035, 84242, 55304, 52999, or 21999 activity is desirable in situations in which 8035, 84242, 55304, 52999, or 21999 is downregulated and/or in which increased 8035, 84242, 55304, 52999, or 21999 activity is likely to have a beneficial effect. Likewise, inhibition of 8035, 84242, 55304, 52999, or 21999 activity is desirable in situations in which 8035, 84242, 55304, 52999, or 21999 is abnormally upregulated and/or in which decreased 8035, 84242, 55304, 52999, or 21999 activity is likely to have a beneficial effect.

The 8035, 84242, 55304, 52999, or 21999 molecules can act as novel diagnostic targets and therapeutic agents for controlling one or more of cellular proliferative and/or differentiative disorders, viral diseases, or metabolic disorders.

Aberrant expression and/or activity of 8035 and 84242 molecules may lead to disorders resulting from aberrant cellular proliferation and/or differentiation including diseases such as cancer, acute promyelocytic leukemia (APL), VHL disease, and systemic lupus erythematosus. In addition, RING finger protein family members such as 8035 and 84242 have been shown to contribute to the pathogenesis of certain viral diseases including those caused by HSV and HIV.

The 55304 molecules can act as novel diagnostic targets and therapeutic agents for controlling one or more of cellular proliferative and/or differentiative disorders, hypertensive disorders, hormone disorders, and disorders associated with protein maturation as described above, as well as disorders associated with bone metabolism, hematopoietic disorders, liver disorders, viral diseases, pain or metabolic disorders.

Aberrant expression and/or activity of 55304 molecules may mediate disorders associated with bone metabolism. “Bone metabolism” refers to direct or indirect effects in the formation or degeneration of bone structures, e.g., bone formation, bone resorption, etc., which may ultimately affect the concentrations in serum of calcium and phosphate. This term also includes activities mediated by 55304 molecules effects in bone cells, e.g. osteoclasts and osteoblasts, that may in turn result in bone formation and degeneration. For example, 55304 molecules may support different activities of bone resorbing osteoclasts such as the stimulation of differentiation of monocytes and mononuclear phagocytes into osteoclasts. Accordingly, 55304 molecules that modulate the production of bone cells can influence bone formation and degeneration, and thus may be used to treat bone disorders. Examples of such disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

Examples of hematopoietic disorders include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjögren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions,leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

Disorders which may be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. The methods described herein can be used to diagnose or treat hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). For example, the methods can be used for the early detection of hepatic injury, such as portal hypertension or hepatic fibrosis. In addition, the methods can be employed to detect liver fibrosis attributed to inborn errors of metabolism, for example, fibrosis resulting from a storage disorder such as Gaucher's disease (lipid abnormalities) or a glycogen storage disease, A1-antitrypsin deficiency; a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome). Additionally, the methods described herein may be useful for the early detection and treatment of liver injury associated with the administration of various chemicals or drugs, such as for example, methotrexate, isonizaid, oxyphenisatin, methyldopa, chlorpromazine, tolbutamide or alcohol, or which represents a hepatic manifestation of a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

Additionally, 55304 molecules may play an important role in the etiology of certain viral diseases, including but not limited to, Hepatitis B, Hepatitis C and Herpes Simplex Virus (HSV). Modulators of 55304 activity could be used to control viral diseases. The modulators can be used in the treatment and/or diagnosis of viral infected tissue or virus-associated tissue fibrosis, especially liver and liver fibrosis. Also, 55304 modulators can be used in the treatment and/or diagnosis of virus-associated carcinoma, especially hepatocellular cancer.

Additionally, 55304 may play an important role in the regulation of metabolism or pain disorders. Diseases of metabolic imbalance include, but are not limited to, obesity, anorexia nervosa, cachexia, lipid disorders, and diabetes. Examples of pain disorders include, but are not limited to, pain response elicited during various forms of tissue injury, e.g., inflammation, infection, and ischemia, usually referred to as hyperalgesia (described in, for example, Fields, H. L. (1987) Pain, New York:McGraw-Hill); pain associated with musculoskeletal disorders, e.g., joint pain; tooth pain; headaches; pain associated with surgery; pain related to irritable bowel syndrome; or chest pain.

The 52999 molecules can act as novel diagnostic targets and therapeutic agents for controlling one or more of cellular proliferative and/or differentiative disorders; inflammatory disorders including, but not limited to osteoarthritis and rheumatoid arthritis, multiple sclerosis, Crohn disease, psoriasis, periodontal disease, and asthma; macular degeneration; restenosis; and Alzheimer's disease.

Similarly, aberrant expression and/or activity of 52999 molecules may mediate disorders associated with, for example, hematopoietic disorders including, but not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjögren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions,leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

The 21999 molecules can act as novel diagnostic targets and therapeutic agents for controlling one or more of cellular proliferative and/or differentiative disorders, cardiovascular disorders, as described above, as well as disorders associated with hematopoietic disorders, liver disorders, viral diseases, or metabolic disorders. Examples of these disorders may be found above.

Pharmacogenomics

The 8035, 84242, 55304, 52999, or 21999 molecules of the present invention, as well as agents, or modulators which have a stimulatory or inhibitory effect on 8035, 84242, 55304, 52999, or 21999 activity (e.g., 8035, 84242, 55304, 52999, or 21999 gene expression) as identified by a screening assay described herein can be administered to individuals to treat (prophylactically or therapeutically) 8035, 84242, 55304, 52999, or 21999 associated disorders (e.g., cellular growth related disorders) associated with aberrant or unwanted 8035, 84242, 55304, 52999, or 21999 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an 8035, 84242, 55304, 52999, or 21999 molecule or an 8035, 84242, 55304, 52999, or 21999 modulator as well as tailoring the dosage and/or therapeutic regimen of treatment with an 8035, 84242, 55304, 52999, or 21999 molecule or 8035, 84242, 55304, 52999, or 21999 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2): 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high-resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., an 8035, 84242, 55304, 52999, or 21999 protein of the present invention), all common variants of that gene can be fairly easily identified in the population and it can be determined if having one version of the gene versus another is associated with a particular drug response.

Alternatively, a method termed the “gene expression profiling”, can be utilized to identify genes that predict drug response. For example, the gene expression of an animal dosed with a drug (e.g., an 8035, 84242, 55304, 52999, or 21999 molecule or 8035, 84242, 55304, 52999, or 21999 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with an 8035, 84242, 55304, 52999, or 21999 molecule or 8035, 84242, 55304, 52999, or 21999 modulator, such as a modulator identified by one of the exemplary screening assays described herein.

The present invention further provides methods for identifying new agents, or combinations, that are based on identifying agents that modulate the activity of one or more of the gene products encoded by one or more of the 8035, 84242, 55304, 52999, or 21999 genes of the present invention, wherein these products may be associated with resistance of the cells to a therapeutic agent. Specifically, the activity of the proteins encoded by the 8035, 84242, 55304, 52999, or 21999 genes of the present invention can be used as a basis for identifying agents for overcoming agent resistance. By blocking the activity of one or more of the resistance proteins, target cells, e.g., cancer cells, will become sensitive to treatment with an agent that the unmodified target cells were resistant to.

Monitoring the influence of agents (e.g., drugs) on the expression or activity of a 8035, 84242, 55304, 52999, or 21999 protein can be applied in clinical trials. For example, the effectiveness of an agent determined by a screening assay as described herein to increase 8035, 84242, 55304, 52999, or 21999 gene expression, protein levels, or upregulate 8035, 84242, 55304, 52999, or 21999 activity, can be monitored in clinical trials of subjects exhibiting decreased 8035, 84242, 55304, 52999, or 21999 gene expression, protein levels, or downregulated 8035, 84242, 55304, 52999, or 21999 activity, respectively. Alternatively, the effectiveness of an agent determined by a screening assay to decrease 8035, 84242, 55304, 52999, or 21999 gene expression, protein levels, or downregulate 8035, 84242, 55304, 52999, or 21999 activity, can be monitored in clinical trials of subjects exhibiting increased 8035, 84242, 55304, 52999, or 21999 gene expression, protein levels, or upregulated 8035, 84242, 55304, 52999, or 21999 activity, respectively. In such clinical trials, the expression or activity of an 8035, 84242, 55304, 52999, or 21999 gene, and preferably, other genes that have been implicated in, for example, an 8035, 84242, 55304, 52999, or 21999-associated disorder can be used as a “read out” or markers of the phenotype of a particular cell.

Other Embodiments

In another aspect, the invention features, a method of analyzing a plurality of capture probes. The method can be used, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence; contacting the array with an 8035, 84242, 55304, 52999, or 21999, preferably purified, nucleic acid, preferably purified, polypeptide, preferably purified, or antibody, and thereby evaluating the plurality of capture probes. Binding, e.g., in the case of a nucleic acid, hybridization with a capture probe at an address of the plurality, is detected, e.g., by signal generated from a label attached to the 8035, 84242, 55304, 52999, or 21999 nucleic acid, polypeptide, or antibody.

The capture probes can be a set of nucleic acids from a selected sample, e.g., a sample of nucleic acids derived from a control or non-stimulated tissue or cell.

The method can include contacting the 8035, 84242, 55304, 52999, or 21999 nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of 8035, 84242, 55304, 52999, or 21999. Such methods can be used to diagnose a subject, e.g., to evaluate risk for a disease or disorder, to evaluate suitability of a selected treatment for a subject, to evaluate whether a subject has a disease or disorder. 8035 and 84242 are associated with RING finger protein activity, thus it is useful for disorders associated with abnormal cellular proliferation and/or differentiation. 55304 is associated with aminopeptidase activity, thus it is useful for disorders associated with abnormal lipid metabolism. 52999 is associated with metallopeptidase activity, thus it, too, is useful for disorders associated with abnormal lipid metabolism.

The method can be used to detect SNPs, as described above.

In another aspect, the invention features, a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express or misexpress 8035, 84242, 55304, 52999, or 21999 or from a cell or subject in which an 8035, 84242, 55304, 52999, or 21999 mediated response has been elicited, e.g., by contact of the cell with 8035, 84242, 55304, 52999, or 21999 nucleic acid or protein, or administration to the cell or subject 8035, 84242, 55304, 52999, or 21999 nucleic acid or protein; contacting the array with one or more inquiry probe, wherein an inquiry probe can be a nucleic acid, polypeptide, or antibody (which is preferably other than 8035, 84242, 55304, 52999, or 21999 nucleic acid, polypeptide, or antibody); providing a two dimensional array having a plurality of addresses, each address of the plurality being positionally distinguishable from each other address of the plurality, and each address of the plurality having a unique capture probe, e.g., wherein the capture probes are from a cell or subject which does not express 8035, 84242, 55304, 52999, or 21999 (or does not express as highly as in the case of the 8035, 84242, 55304, 52999, or 21999 positive plurality of capture probes) or from a cell or subject which in which an 8035, 84242, 55304, 52999, or 21999 mediated response has not been elicited (or has been elicited to a lesser extent than in the first sample); contacting the array with one or more inquiry probes (which is preferably other than an 8035, 84242, 55304, 52999, or 21999 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features, a method of analyzing 8035, 84242, 55304, 52999, or 21999, e.g., analyzing structure, function, or relatedness to other nucleic acid or amino acid sequences. The method includes: providing an 8035, 84242, 55304, 52999, or 21999 nucleic acid or amino acid sequence; comparing the 8035, 84242, 55304, 52999, or 21999 sequence with one or more preferably a plurality of sequences from a collection of sequences, e.g., a nucleic acid or protein sequence database; to thereby analyze 8035, 84242, 55304, 52999, or 21999.

Preferred databases include GenBank™. The method can include evaluating the sequence identity between an 8035, 84242, 55304, 52999, or 21999 sequence and a database sequence. The method can be performed by accessing the database at a second site, e.g., over the internet.

In another aspect, the invention features, a set of oligonucleotides, useful, e.g., for identifying SNP's, or identifying specific alleles of 8035, 84242, 55304, 52999, or 21999. The set includes a plurality of oligonucleotides, each of which has a different nucleotide at an interrogation position, e.g., an SNP or the site of a mutation. In a preferred embodiment, the oligonucleotides of the plurality identical in sequence with one another (except for differences in length). The oligonucleotides can be provided with different labels, such that an oligonucleotides which hybridizes to one allele provides a signal that is distinguishable from an oligonucleotides which hybridizes to a second allele.

This invention is further illustrated by the following examples which should not be construed as limiting. The contents of all references, patents and published patent applications cited throughout this application are incorporated herein by reference.

### EXAMPLES

### Example 1

### Identification and Characterization of Human 8035 and 84242 cDNAs

The human 8035 sequence (FIG. 1; SEQ ID NO: 1), which is approximately 2876 nucleotides long including untranslated regions, contains a predicted methionine-initiated coding sequence of about 1299 nucleotides (nucleotides 613-1914 of SEQ ID NO: 1; SEQ ID NO: 3). The coding sequence encodes a 433 amino acid protein (SEQ ID NO: 2).

The human 84242 sequence (FIG. 2; SEQ ID NO: 5), which is approximately 2810 nucleotides long including untranslated regions, contains a predicted methionine-initiated coding sequence of about 1209 nucleotides (nucleotides 744-1955 of SEQ ID NO: 5; SEQ ID NO: 7). The coding sequence encodes a 403 amino acid protein (SEQ ID NO: 6).

### Example 2

### Distribution of Human 84242 in Various Cells and Tissues

Human 84242 showed elevated levels of expression in coronary smooth muscle, human umbilical vein endothelia, heart, kidney, skeletal muscle, brain, dorsal root ganglion, breast, prostate, colon, lung, skin, bone marrow, blood, and erythroid cells and tissues. See Table 2. Expression levels of 84242 were observed to be higher in tumors of the breast, prostate, colon, and lung, relative to normal tissue. See Table 2, rows 22, 26, 29, and 31.

TABLE 2
Expression of 84242 in various cells and tissues.
PHASE 1.5.1 EXPRESSION OF 84242(F1R1)

| | | RELATIVE |
|---|---|---|
| | TISSUE TYPE | EXPRESSION |
| 1 | Artery normal | 9.2907 |
| 2 | Aorta diseased | 8.2294 |
| 3 | Vein normal | 2.9399 |
| 4 | Coronary SMC (Smooth Muscle Cell) | 12.4734 |
| 5 | HUVEC (Human Umbilical Vein Endothelial Cells) | 40.9498 |
| 6 | Hemangioma | 3.5697 |
| 7 | Heart normal | 11.6381 |
| 8 | Heart CHF (Congestive Heart Failure) | 12.6038 |
| 9 | Kidney | 11.5577 |
| 10 | Skeletal Muscle | 18.6459 |
| 11 | Adipose normal | 2.7717 |
| 12 | Pancreas | 4.8259 |
| 13 | primary osteoblasts | 9.1946 |
| 14 | Osteoclasts (Differentiated) | 2.9913 |
| 15 | Skin normal | 6.2584 |
| 16 | Spinal cord normal | 4.3043 |
| 17 | Brain Cortex normal | 50.942 |
| 18 | Brain Hypothalamus, normal | 9.9575 |
| 19 | Nerve | 5.8393 |
| 20 | DRG (Dorsal Root Ganglion) | 12.8241 |
| 21 | Breast normal | 4.6453 |
| 22 | Breast tumor | 10.1667 |
| 23 | Ovary normal | 8.5789 |
| 24 | Ovary Tumor | 3.0968 |
| 25 | Prostate Normal | 4.5027 |
| 26 | Prostate Tumor | 12.0904 |
| 27 | Salivary glands | 1.816 |
| 28 | Colon normal | 6.9682 |
| 29 | Colon Tumor | 20.0535 |
| 30 | Lung normal | 4.4253 |
| 31 | Lung tumor | 21.7175 |
| 32 | Lung COPD (Chronic Obstructive Pulmonary | 6.0872 |
| | Disease) | |
| 33 | Colon IBD (Inflammatory Bowel Disease) | 7.8125 |
| 34 | Liver normal | 2.7241 |
| 35 | Liver fibrosis | 4.9102 |
| 36 | Spleen normal | 2.5241 |
| 37 | Tonsil normal | 8.7591 |
| 38 | Lymph node, normal | 4.4253 |
| 39 | Small intestine normal | 6.5241 |
| 40 | Skin-Decubitus | 12.7355 |
| 41 | Synovium | 2.4129 |
| 42 | BM-MNC (Bone Marrow-Mononuclear Cell) | 15.0405 |
| 43 | Activated PBMC (Peripheral Blood Mononuclear | 15.2505 |
| | Cell) | |
| 44 | Neutrophils | 3.2848 |
| 45 | Megakaryocytes | 4.996 |
| 46 | Erythroid | 17.337 |

Expression levels were determined by quantitative PCR (Taqman® brand quantitative PCR kit, Applied Biosystems). The quantitative PCR reactions were performed according to the kit manufacturer's instructions.

### Example 3

### Identification and Characterization of Human 55304 cDNAs

The human 55304 sequence (FIG. 7A-B; SEQ ID NO: 9), which is approximately 5502 nucleotides long including untranslated regions, contains a predicted methionine-initiated coding sequence of about 2039 nucleotides (nucleotides 803-2845 of SEQ ID NO: 11). The coding sequence encodes a 680 amino acid protein (SEQ ID NO: 10).

### Example 4

### Distribution of Human 55304 in Various Cells and Tissues

Human 555304 showed elevated levels of expression in brain, kidney, testes, and epithelial cells of the prostate. See Tables 3 and 4.

TABLE 3
Expression of 55304 in various cells and tissues.
55304 HUMAN PANEL PHASE I

| | | RELATIVE |
|---|---|---|
| | TISSUE | EXPRESSION |
| 1 | Adrenal Gland | 0.95 |
| 2 | Brain | 2.66 |
| 3 | Heart | 0.32 |
| 4 | Kidney | 3.66 |
| 5 | Liver | 0.12 |
| 6 | Lung | 0.15 |
| 7 | Mammary Gland | 0.25 |
| 8 | Placenta | 1.18 |
| 9 | Prostate | 0.68 |
| 10 | Salivary Gland | 1.55 |
| 11 | Muscle | 0.74 |
| 12 | Sm. Intestine | 0.42 |
| 13 | Spleen | 0.08 |
| 14 | Stomach | 0.68 |
| 15 | Testes | 5 |
| 16 | Thymus | 0.34 |
| 17 | Trachea | 0.45 |
| 18 | Uterus | 0.16 |
| 19 | Spinal Cord | 1.19 |
| 20 | Skin | 0.20 |
| 21 | DRG (Dorsal Root Ganglion) | 0.45 |

Expression levels of 55304 were observed to be higher in tumors of the colon and lung, relative to normal tissue. See Table 4, rows 31 and 33.

TABLE 4
Expression of 55304 in various cells and tissues.
PHASE 1.3.3 EXPRESSION OF 55304.1

| | | RELATIVE |
|---|---|---|
| | TISSUE TYPE | EXPRESSION |
| 1 | Artery normal | 0 |
| 2 | Vein normal | 0 |
| 3 | Aortic SMC (Smooth Muscle Cell) EARLY | 1.1981 |
| 4 | Coronary SMC | 2.022 |
| 5 | Static HUVEC (Human Umbilical Vein Endothelial | 1.4497 |
| | Cell) | |
| 6 | Shear HUVEC | 0.6465 |
| 7 | Heart normal | 0.2366 |
| 8 | Heart CHF (Chronic Heart Failure) | 1.736 |
| 9 | Kidney | 38.8754 |
| 10 | Skeletal Muscle | 0.1922 |
| 11 | Adipose normal | 0 |
| 12 | Pancreas | 1.1613 |
| 13 | Primary Osteoblasts | 0.0619 |
| 14 | Osteoclasts (differentiated) | 0.0143 |
| 15 | Skin normal | 0.4192 |
| 16 | Spinal cord normal | 3.1619 |
| 17 | Brain Cortex normal | 22.5614 |
| 18 | Brain Hypothalamus normal | 11.8415 |
| 19 | Nerve | 1.1063 |
| 20 | DRG (Dorsal Root Ganglion) | 3.472 |
| 21 | Glial Cells (Astrocytes) | 3.2395 |
| 22 | Glioblastoma | 0.2814 |
| 23 | Breast normal | 0.0458 |
| 24 | Breast tumor | 0.3818 |
| 25 | Ovary normal | 5.3546 |
| 26 | Ovary Tumor | 1.1735 |
| 27 | Prostate Normal | 0.5003 |
| 28 | Prostate Tumor | 0.1529 |
| 29 | Epithelial Cells (Prostate) | 40.2463 |
| 30 | Colon normal | 0.1002 |
| 31 | Colon Tumor | 8.6385 |
| 32 | Lung normal | 0 |
| 33 | Lung tumor | 4.9444 |
| 34 | Lung COPD (Chronic Obstructive Pulmonary | 0.0954 |
| | Disorder) | |
| 35 | Colon IBD (Inflammatory Bowel Disease) | 0.0903 |
| 36 | Liver normal | 0.309 |
| 37 | Liver fibrosis | 0.4733 |
| 38 | Dermal Cells - fibroblasts | 0.2416 |
| 39 | Spleen normal | 0.059 |
| 40 | Tonsil normal | 0.6159 |
| 41 | Lymphnode | 0.4684 |
| 42 | Small Intestine | 0.1299 |
| 43 | Skin-Decubitus | 0.5108 |
| 44 | Synovium | 0.0287 |
| 45 | BM-MNC (Bone Marrow Mononuclear Cell) | 0.6095 |
| 46 | Activated PBMC (Peripheral Blood Mononuclear | 0.0125 |
| | Cell) | |

Expression levels were determined as described in Example 3.

### Example 5

### Identification and Characterization of Human 52999 cDNAs

The human 52999 sequence (FIG. 9A-B; SEQ ID NO: 12), which is approximately 2566 nucleotides long including untranslated regions, contains a predicted methionine-initiated coding sequence of about 2277 nucleotides (nucleotides 194-2470 of SEQ ID NO: 12; SEQ ID NO: 14). The coding sequence encodes a 758 amino acid protein (SEQ ID NO: 13).

### Example 6

### Identification and Characterization of 21999 Human ADP-ribosyltransferase cDNAs

The human ADP-ribosyltransferase sequence (FIG. 12; SEQ ID NO: 19), which is approximately 1485 nucleotides long including untranslated regions, contains a predicted methionine-initiated coding sequence of about 876 nucleotides (nucleotides 255-1133 of SEQ ID NO: 19). The coding sequence encodes a 292 amino acid protein (SEQ ID NO: 20).

### Example 7

### Distribution of Human 21999 in Various Cells and Tissues

Human 21999 showed elevated levels of expression in skeletal muscle and ovary tissue. See Table 5.

TABLE 5
Expression of 21999 in various cells and tissues.
PHASE 1.6.5 OF 21999

| | | RELATIVE |
|---|---|---|
| | TISSUE TYPE | EXPRESSION |
| 1 | Artery normal | 0 |
| 2 | Aorta diseased | 0 |
| 3 | Vein normal | 0 |
| 4 | Coronary SMC (Smooth Muscle Cell) | 0 |
| 5 | HUVEC (Human Umbilical Vein Endothelial Cell) | 0 |
| 6 | Hemangioma | 0 |
| 7 | Heart normal | 0 |
| 8 | Heart CHF (Chronic Heart Failure) | 0.1044 |
| 9 | Kidney | 0 |
| 10 | Skeletal Muscle | 1.5218 |
| 11 | Adipose normal | 0 |
| 12 | Pancreas | 0 |
| 13 | Primary Osteoblasts | 0 |
| 14 | Osteoclasts (differentiated) | 0 |
| 15 | Skin normal | 0 |
| 16 | Spinal cord normal | 0 |
| 17 | Brain Cortex normal | 0 |
| 18 | Brain Hypothalamus normal | 0 |
| 19 | Nerve | 0 |
| 20 | DRG (Dorsal Root Ganglion) | 0 |
| 21 | Breast normal | 0.1482 |
| 22 | Breast tumor | 0 |
| 23 | Ovary normal | 1.2191 |
| 24 | Ovary Tumor | 0.0332 |
| 25 | Prostate Normal | 0.0641 |
| 26 | Prostate Tumor | 0 |
| 27 | Salivary glands | 0 |
| 28 | Colon normal | 0 |
| 29 | Colon Tumor | 0 |
| 30 | Lung normal | 0.0202 |
| 31 | Lung tumor | 0.871 |
| 32 | Lung COPD (Chronic Obstructive Pulmonary | 0 |
| | Disease) | |
| 33 | Colon IBD (Inflammatory Bowel Disease) | 0 |
| 34 | Liver normal | 0 |
| 35 | Liver fibrosis | 0 |
| 36 | Spleen normal | 0 |
| 37 | Tonsil normal | 0 |
| 38 | Lymph node normal | 0 |
| 39 | Small intestine normal | 0 |
| 40 | Macrophages | 0 |
| 41 | Synovium | 0 |
| 42 | BM-MNC (Bone-Marrow Mononuclear Cells) | 0 |
| 43 | Activated PBMC (Peripheral Blood Mononuclear | 0 |
| | Cells) | |
| 44 | Neutrophils | 0 |
| 45 | Megakaryocytes | 0 |
| 46 | Erythroid | 0 |
| 47 | Positive Control | 10.5253 |

Expression levels of 21999 were observed to be higher in tumors of the lung and colon, as well as in metastatic liver, relative to normal tissue. See Table 6, rows 22, 26, 30, 34, and 35. 21999 expression was also elevated in both normoxic and hypoxic colon tumor cell lines (HCT116). Table 6, rows 42 and 43.

TABLE 6
Expression of 21999 in various cells and tissues.
21999.1 ONCOLOGY PHASE II PANEL

| | | RELATIVE |
|---|---|---|
| | TISSUE TYPE | EXPRESSION |
| 1 | PIT 400 Breast Normal | 0.00 |
| 2 | PIT 372 Breast Normal | 3.15 |
| 3 | CHT 1228 Breast Normal | 1.31 |
| 4 | MDA 304 Breast Tumor: MD-IDC (Moderately | 0.00 |
| | Differentiated-Invasive Ductal Carcinoma) | |
| 5 | CHT 2002 Breast Tumor: DC | 0.00 |
| 6 | MDA 236-Breast Tumor: PD-IDC(ILC?) (Poorly | 0.00 |
| | Differentiated-IDC(Invasive Lobular Carcinoma?)) | |
| 7 | CHT 562 Breast Tumor: IDC | 0.00 |
| 8 | NDR 138 Breast Tumor ILC (LG) | 0.00 |
| 9 | CHT 1841 Lymph node (Breast metastases) | 0.00 |
| 10 | PIT 58 Lung (Breast metastases) | 0.00 |
| 11 | CHT 620 Ovary Normal | 14.38 |
| 12 | PIT 208 Ovary Normal | 6.39 |
| 13 | CLN 012 Ovary Tumor | 96.05 |
| 14 | CLN 07 Ovary Tumor | 0.07 |
| 15 | CLN 17 Ovary Tumor | 9.82 |
| 16 | MDA 25 Ovary Tumor | 0.35 |
| 17 | CLN 08 Ovary Tumor | 0.48 |
| 18 | PIT 298 Lung Normal | 0.05 |
| 19 | MDA 185 Lung Normal | 0.00 |
| 20 | CLN 930 Lung Normal | 0.57 |
| 21 | MPI 215 Lung Tumor--SmC | 0.00 |
| 22 | MDA 259 Lung Tumor-PDNSCCL | 32.13 |
| 23 | CHT 832 Lung Tumor-PDNSCCL | 0.00 |
| 24 | MDA 262 Lung Tumor-SCC (Squamous Cell | 1.01 |
| | Carcinoma) | |
| 25 | CHT 793 Lung Tumor-ACA | 0.00 |
| 26 | CHT 331 Lung Tumor-ACA | 14.63 |
| 27 | CHT 405 Colon Normal | 0.00 |
| 28 | CHT 523 Colon Normal | 0.20 |
| 29 | CHT 371 Colon Normal | 0.00 |
| 30 | CHT 382 Colon Tumor: MD | 8.46 |
| 31 | CHT 528 Colon Tumor: MD | 0.17 |
| 32 | CLN 609 Colon Tumor | 0.13 |
| 33 | NDR 210 Colon Tumor: MD-PD | 0.00 |
| 34 | CHT 340 Colon-Liver Metastases | 6.19 |
| 35 | CHT 1637 Colon-Liver Metastases | 2.14 |
| 36 | PIT 260 Liver Normal (female) | 0.00 |
| 37 | CHT 1653 Cervix Squamous CC | 0.00 |
| 38 | CHT 569 Cervix Squamous CC | 0.00 |
| 39 | A24 HMVEC (Human Microvessel Endothelial | 0.00 |
| | Cell)-Arresting | |
| 40 | C48 HMVEC-Proliferating | 0.00 |
| 41 | Pooled Hemangiomas | 0.00 |
| 42 | HCT116N22 Normoxic | 51.30 |
| 43 | HCT116H22 Hypoxic | 47.37 |

Expression levels were determined as described in Example 3.

### Example 8

### Tissue Distribution of 8035, 84242, 55304, 52999, or 21999 mRNA

Northern blot hybridizations with various RNA samples can be performed under standard conditions and washed under stringent conditions, i.e., 0.2×SSC at 65° C. A DNA probe corresponding to all or a portion of the 8035, 84242, 55304, 52999, or 21999 cDNA (SEQ ID NO: 1, SEQ ID NO: 5, SEQ ID NO: 9, SEQ ID NO: 12, or SEQ ID NO: 19) can be used. The DNA is radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) can be probed in ExpressHyb hybridization solution (Clontech) and washed at high stringency according to manufacturer's recommendations.

### Example 9

### Recombinant Expression of 8035. 84242. 55304. 52999. or 21999 in Bacterial Cells

In this example, 8035, 84242, 55304, 52999, or 21999 are expressed as recombinant glutathione-S-transferase (GST) fusion polypeptides in E. coli and the fusion polypeptides are isolated and characterized. Specifically, 8035, 84242, 55304, 52999, or 21999 are fused to GST and these fusion polypeptides are expressed in E. coli, e.g., strain PEB199. Expression of the GST-8035, 84242, 55304, 52999, or 21999 fusion proteins in PEB199 is induced with IPTG. The recombinant fusion polypeptides are purified from crude bacterial lysates of the induced PEB199 strain by affinity chromatography on glutathione beads. Using polyacrylamide gel electrophoretic analysis of the polypeptides purified from the bacterial lysates, the molecular weight of the resultant fusion polypeptides are determined.

### Example 10

### Expression of Recombinant 8035, 84242, 55304, 52999, or 21999 Protein in COS Cells

To express the 8035, 84242, 55304, 52999, or 21999 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. DNA fragments encoding the entire 8035, 84242, 55304, 52999, or 21999 proteins and an HA tag (Wilson et al. (1984) Cell 37:767) or a FLAG tag fused in-frame to its 3′ end of each fragment are cloned into the polylinker region of the each vector, thereby placing the expression of the recombinant proteins under the control of the CMV promoter.

To construct the plasmids, the 8035, 84242, 55304, 52999, or 21999 DNA sequences are amplified by PCR using two primers for each. The 5′ primer contains the restriction site of interest followed by approximately twenty nucleotides of the 8035, 84242, 55304, 52999, or 21999 coding sequence starting from the initiation codon; the 3′ end sequence contains complementary sequences to the other restriction site of interest, a translation stop codon, the HA tag or FLAG tag and the last 20 nucleotides of the 8035, 84242, 55304, 52999, or 21999 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the 8035, 84242, 55304, 52999, or 21999 genes are inserted in the correct orientation. The ligation mixtures are transformed into E. coli cells (strains HB 101, DH5α, SURE, available from Stratagene Cloning Systems, La Jolla, Calif., can be used), the transformed cultures are plated on ampicillin media plates, and resistant colonies are selected. Plasmid DNA is isolated from transformants and examined by restriction analysis for the presence of the correct fragments.

COS cells are subsequently transfected with the 8035, 84242, 55304, 52999, or 21999-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the 8035, 84242, 55304, 52999, or 21999 polypeptides is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 8035, 84242, 55304, 52999, or 21999 coding sequence is cloned directly into the polylinker of the pCDNA/Amp vector using the appropriate restriction sites. The resulting plasmid is transfected into COS cells in the manner described above, and the expression of the 8035, 84242, 55304, 52999, or 21999 polypeptide is detected by radiolabelling and immunoprecipitation using an 8035, 84242, 55304, 52999, or 21999-specific monoclonal antibody, respectively.

### Equivalents

Those skilled in the art will recognize, or be able to ascertain using no more than routine experimentation, many equivalents to the specific embodiments of the invention described herein. Such equivalents are intended to be encompassed by the following claims.

That which is claimed:

1. An isolated nucleic acid molecule selected from the group consisting of:
a) a nucleic acid molecule comprising a nucleotide sequence which is at least 70% identical to the nucleotide sequence of SEQ ID NO: 1 or SEQ ID NO: 3, SEQ ID NO: 5 or SEQ ID NO: 7, SEQ ID NO: 9 or SEQ ID NO: 11, SEQ ID NO: 12 or SEQ ID NO: 14, or SEQ ID NO: 19 or SEQ ID NO: 21;
b) a nucleic acid molecule comprising a fragment of at least 300 nucleotides of the nucleotide sequence of SEQ ID NO: 1 or SEQ ID NO: 3, SEQ ID NO: 5 or SEQ ID NO: 7, SEQ ID NO: 9 or SEQ ID NO: 11, SEQ ID NO: 12 or SEQ ID NO: 14, or SEQ ID NO: 19 or SEQ ID NO: 21;
c) a nucleic acid molecule which encodes a polypeptide comprising the amino acid sequence of SEQ ID NO: 2, SEQ ID NO: 6, SEQ ID NO: 10, SEQ ID NO: 13, OR SEQ ID NO: 20;
d) a nucleic acid molecule which encodes a fragment of a polypeptide comprising the amino acid sequence of SEQ ID NO: 2, SEQ ID NO: 6, SEQ ID NO: 10, SEQ ID NO: 13, OR SEQ ID NO: 20, wherein the fragment comprises at least 15 contiguous amino acids of SEQ ID NO: 2, SEQ ID NO: 6, SEQ ID NO: 10, SEQ ID NO: 13, OR SEQ ID NO: 20, respectively; and
e) a nucleic acid molecule which encodes a naturally occurring allelic variant of a polypeptide comprising the amino acid sequence of SEQ ID NO: 2, SEQ ID NO: 6, SEQ ID NO: 10, SEQ ID NO: 13, OR SEQ ID NO: 20, wherein the nucleic acid molecule hybridizes to a nucleic acid molecule comprising SEQ ID NO: 1 or SEQ ID NO: 3, SEQ ID NO: 5 or SEQ ID NO: 7, SEQ ID NO: 9 or SEQ ID NO: 11, SEQ ID NO: 12 or SEQ ID NO: 14, or SEQ ID NO: 19 or SEQ ID NO: 21, respectively, or a complement thereof, under stringent conditions.

2. The isolated nucleic acid molecule of claim 1, which is selected from the group consisting of:
a) a nucleic acid comprising the nucleotide sequence of SEQ ID NO: 1 or SEQ ID NO: 3, SEQ ID NO: 5 or SEQ ID NO: 7, SEQ ID NO: 9 or SEQ ID NO: 11, SEQ ID NO: 12 or SEQ ID NO: 14, or SEQ ID NO: 19 or SEQ ID NO: 21; and
b) a nucleic acid molecule which encodes a polypeptide comprising the amino acid sequence of SEQ ID NO: 2, SEQ ID NO: 6, SEQ ID NO: 10, SEQ ID NO: 13, OR SEQ ID NO: 20.

3. The nucleic acid molecule of claim 1 further comprising vector nucleic acid sequences.

4. The nucleic acid molecule of claim 1 further comprising nucleic acid sequences encoding a heterologous polypeptide.

5. A host cell which contains the nucleic acid molecule of claim 3.

6. The host cell of claim 5 which is a mammalian host cell.

7. A non-human mammalian host cell containing the nucleic acid molecule of claim 3.

8. An isolated polypeptide selected from the group consisting of:
a) a polypeptide which is encoded by a nucleic acid molecule comprising a nucleotide sequence which is at least 70% identical to a nucleic acid comprising the nucleotide sequence of SEQ ID NO: 1 or SEQ ID NO: 3, SEQ ID NO: 5 or SEQ ID NO: 7, SEQ ID NO: 9 or SEQ ID NO: 11, SEQ ID NO: 12 or SEQ ID NO: 14, or SEQ ID NO: 19 or SEQ ID NO: 21, or a complement thereof;
b) a naturally occurring allelic variant of a polypeptide comprising the amino acid sequence of SEQ ID NO: 2, SEQ ID NO: 6, SEQ ID NO: 10, SEQ ID NO: 13, OR SEQ ID NO: 20, wherein the polypeptide is encoded by a nucleic acid molecule which hybridizes to a nucleic acid molecule comprising SEQ ID NO: 1 or SEQ ID NO: 3, SEQ ID NO: 5 or SEQ ID NO: 7, SEQ ID NO: 9 or SEQ ID NO: 11, SEQ ID NO: 12 or SEQ ID NO: 14, or SEQ ID NO: 19 or SEQ ID NO: 21, respectively; and
c) a fragment of a polypeptide comprising the amino acid sequence of SEQ ID NO: 2, SEQ ID NO: 6, SEQ ID NO: 10, SEQ ID NO: 13, OR SEQ ID NO: 20, wherein the fragment comprises at least 15 contiguous amino acids of SEQ ID NO: 2, SEQ ID NO: 6, SEQ ID NO: 10, SEQ ID NO: 13, OR SEQ ID NO: 20.

9. The isolated polypeptide of claim 8 comprising the amino acid sequence of SEQ ID NO: 2, SEQ ID NO: 6, SEQ ID NO: 10, SEQ ID NO: 13, OR SEQ ID NO: 20.

10. The polypeptide of claim 8 further comprising heterologous amino acid sequences.

11. An antibody which selectively binds to a polypeptide of claim 8.

12. A method for producing a polypeptide selected from the group consisting of:
a) a polypeptide comprising the amino acid sequence of SEQ ID NO: 2, SEQ ID NO: 6, SEQ ID NO: 10, SEQ ID NO: 13, OR SEQ ID NO: 20;
b) a polypeptide comprising a fragment of the amino acid sequence of SEQ ID NO: 2, SEQ ID NO: 6, SEQ ID NO: 10, SEQ ID NO: 13, OR SEQ ID NO: 20, wherein the fragment comprises at least 15 contiguous amino acids of SEQ ID NO: 2, SEQ ID NO: 6, SEQ ID NO: 10, SEQ ID NO: 13, OR SEQ ID NO: 20, respectively; and
c) a naturally occurring allelic variant of a polypeptide comprising the amino acid sequence of SEQ ID NO: 2, SEQ ID NO: 6, SEQ ID NO: 10, SEQ ID NO: 13, OR SEQ ID NO: 20, wherein the polypeptide is encoded by a nucleic acid molecule which hybridizes to a nucleic acid molecule comprising SEQ ID NO: 1 or SEQ ID NO: 3, SEQ ID NO: 5 or SEQ ID NO: 7, SEQ ID NO: 9 or SEQ ID NO: 11, SEQ ID NO: 12 or SEQ ID NO: 14, or SEQ ID NO: 19 or SEQ ID NO: 21, respectively; comprising culturing the host cell of claim 5 under conditions in which the nucleic acid molecule is expressed.

13. A method for detecting the presence of a polypeptide of claim 8 in a sample, comprising:
a) contacting the sample with a compound which selectively binds to a polypeptide of claim 8; and
b) determining whether the compound binds to the polypeptide in the sample.

14. The method of claim 13, wherein the compound which binds to the polypeptide is an antibody.

15. A kit comprising a compound which selectively binds to a polypeptide of claim 8 and instructions for use.

16. A method for detecting the presence of a nucleic acid molecule of claim 1 in a sample, comprising the steps of:
a) contacting the sample with a nucleic acid probe or primer which selectively hybridizes to the nucleic acid molecule; and
b) determining whether the nucleic acid probe or primer binds to a nucleic acid molecule in the sample.

17. The method of claim 16, wherein the sample comprises mRNA molecules and is contacted with a nucleic acid probe.

18. A kit comprising a compound which selectively hybridizes to a nucleic acid molecule of claim 1 and instructions for use.

19. A method for identifying a compound which binds to a polypeptide of claim 8 comprising the steps of:
a) contacting a polypeptide, or a cell expressing a polypeptide of claim 8 with a test compound; and
b) determining whether the polypeptide binds to the test compound.

20. The method of claim 19, wherein the binding of the test compound to the polypeptide is detected by a method selected from the group consisting of:
a) detection of binding by direct detecting of test compound/polypeptide binding;
b) detection of binding using a competition binding assay;
c) detection of binding using an assay for 25233-mediated transamination, decarboxylation, deamination, racemization, or aldol cleavage.

21. A method for modulating the activity of a polypeptide of claim 8 comprising contacting a polypeptide or a cell expressing a polypeptide of claim 8 with a compound which binds to the polypeptide in a sufficient concentration to modulate the activity of the polypeptide.

22. A method for identifying a compound which modulates the activity of a polypeptide of claim 8, comprising:
a) contacting a polypeptide of claim 8 with a test compound; and
b) determining the effect of the test compound on the activity of the polypeptide to thereby identify a compound which modulates the activity of the polypeptide.
